(19) United States
(12) Patent Application Publication
(10) Pub. No.: US 2007/0203159 A1
(43) Pub. Date: Aug. 30, 2007

(54) Purine compounds

(75) Inventors: Lise-Lotte Gundersen, Oslo (NO); Morten Braendvang, Oslo (NO)

Correspondence Address: BACON & THOMAS, PLLC, 625 SLATERS LANE, FOURTH FLOOR, ALEXANDRIA, VA, 22314, US

(73) Assignee: Universitetet i Olso, Oslo (NO)

(21) Appl. No.: 11/362,771

(22) Filed: Feb. 28, 2006

(51) Int. Cl. A61K 31/52; A61K 31/519; C07D 473/12; C07D 471/02; C07D 487/02

(52) U.S. Cl. 514/261.1; 514/263.4; 514/265.1; 514/303; 544/277; 544/280; 546/119; 514/263.23

(57) ABSTRACT

The invention provides an antimycobacterial 6-aryl-9-(m- or p-substituted-benzyl) purine and purine analog compounds.

This invention relates to novel antimycobacterial purine compounds, their properties, compositions containing them and a method of treatment of mycobacterial infection, in particular tuberculosis, using them.

Tuberculosis (TB) is a disease caused by infection by Mycobacterium tuberculosis. Once a fatal disease (a disease with about 50% mortality rate) in the developed world, it dropped from attention with the development of antibacterial agents such as streptomycin over fifty years ago. However today it is estimated that over one third of the world's population is infected with M. tuberculosis and that about thirty million people will die from TB in the next ten years. Moreover, HIV infection has resulted in increasing numbers of TB sufferers in the developed world and has resulted in increased TB morbidity and mortality. Multi-drug resistant strains of M. tuberculosis have appeared and multidrug resistant tuberculosis (MDP-TB) is a growing problem amongst HIV patients. No new anti TB drugs have reached the market since the 1960s and there is an urgent need for new antimycobacterial drugs, especially ones suitable for the treatment of MDR-TB.

We have now found that certain novel purine derivatives, in particular 6-aryl-9-(m- or p-substituted-benzyl)-purines, have antimycobacterial efficacy against M. tuberculosis.

Thus, viewed from one aspect, the invention provides an antimycobacterial 6-aryl-9-(m- or p-substituted-benzyl)purine, 8-aza-purine or 7-deaza-purine compound or a physiologically tolerable salt thereof.

Viewed from a further aspect the invention provides a pharmaceutical composition comprising an antimycobacterial 6-aryl-9-(m- or p-substituted-benzyl)-purine, 8-aza-purine or 7-deaza-purine or a physiologically tolerable salt thereof together with a physiologically tolerable carrier or excipient.

Viewed from a still further aspect the invention provides the use of an antimycobacterial 6-aryl-9-(m- or p-substituted-benzyl)-purine, 8-aza-purine or 7-deaza-purine or a physiologically tolerable salt thereof for the manufacture of a medicament for use in combatting mycobacterial infection, in particular tuberculosis.

Viewed from a yet further aspect the invention provides a method of treatment of a-human or non-human animal (e.g. a mammal) subject to combat mycobacterial infection, said method comprising adminsitering to said subject an effective amount of an antimycobacterial 6-aryl-9-(m- or p-substituted-benzyl)-purine, 8-aza-purine or 7-deaza-purine or a physiologically tolerable salt thereof.

The compounds of the invention will be referred to hereinafter simply as purine compounds. The purine compounds of the invention are substituted at the 6-position by an aryl group. The aryl group is conveniently a 5 to 10 membered ring system, e.g. a monocyclic or fused bicyclic system, in particular one having at least one and especially no more than 4 heteroatoms, e.g. O, N or S. Especially preferably it is carbon-attached and particularly preferably it is a phenyl ring or a five-membered heterocyclic ring attached at the ring carbon adjacent a heteroatom. Thus for example it may be a phenyl, furyl, thienyl, benzofuryl, pyrrolyl, imidazolyl or pyridyl group. Most preferably it is a 2-furyl group. The aryl group may optionally be substituted, e.g. by hydrocarbyl, hydrocarbyloxy, hydrocarbylthio, hydrocarbylamino, halo, cyano, thio, hydroxy, amino, acyl or acyloxy groups. Preferably however it is unsubstituted or substituted by hydrocarbyl groups. 5-membered rings containing a single O or S ring heteroatom are particularly preferred.

The 9-substituent in the purine compounds of the invention is a benzyl group substituted at one, two or three of the meta and para positions by a group which has a negative σ value and a positive π value, e.g. a σ value or >−1.0, preferably >−0.5, e.g. −0.27 to −0.17 [?] and a π value of <2.0, preferably <1.0, e.g. 0.1 to 0.6, for example 0.56, [?]. Typically this may be a halo, cyano, thio, hydroxy, amino or hydrocarbyl group or a hydrocarbyl group attached via an oxygen, sulphur, amino, carbonyl, carbonylamino, aminocarbonyl or SO group. The ortho positions are preferably unsubstituted, but meta and para positions may be substituted by a bridging substituent, e.g. —O—CH₂—O. The substituent is preferably hydrocarbyl, halo, hydrocarbyloxy or hydrocarbylthio, especially hydrocarbyloxy. Examples of preferred substituents include chloro, methyl, methoxy, and benzyloxy; however substitution by alkoxy, especially methoxy, is especially preferred. Particularly preferably only one or two of the meta and para positions are substituted and especially preferably the para position alone is substituted. 4-methoxybenzylamino and 4-acetamido are not preferred substituents. σ and π values for candidate substituent groups may be found for example in Substituent Constants for Correlation Analysis in Chemistry and Biology, Hansch, C.; Leo, A. Wiley, New York, 1979.

The hydrocarbyl substituents may be saturated or unsaturated and linear, branched or cyclic or a combination of these, e.g. aryl, alkyl, alkenyl, alkynyl, aralkyl, aralkenyl, aralkynyl, alkaryl, cyclic alkyl etc, and may themselves be substituted, e.g. by halo (especially F), alkoxy, hydroxy, thio, alkylthio and heteroaryl groups.

Aryl groups, unless otherwise specified preferably are 5 to 10 ring membered mono or bicyclic groups containing up to 4 heteroatoms, e.g. O, N and S atoms.

Alkyl, alkenyl and alkynyl groups and moieties, unless otherwise specified preferably contain up to 10 carbons, more particularly up to 6 carbons.

Halo groups, unless otherwise specified are preferably Cl, F, I or Br, particularly Cl or F.

The methylene group at the 9-position may be substituted, e.g. by a halo or hydrocarbyl group, for example chloro, methyl or phenyl; however it is preferably unsubstituted other than by the m- or p-substituted phenyl group it carries.

The 2-position of the purine ring system in the purine compounds of the invention is preferably unsubstituted or substituted by a nitro group or one of the groups listed as substituents for the m- or p-substituted phenyl group. If substituted however it is preferably substituted by a halo group.

The 8 and 7 positions in the purine compounds of the invention, i.e. the remaining ring atoms of the five membered ring of the purine structure, may if desired be substituted, e.g. by one of the groups listed as substituents for the m- or p-substituted phenyl group. If substituted, the 8-position preferably carries a halo, hydroxy, cyano, thio, hydrocarbyl, hydrocarbyloxy or hydrocarbylthio group, e.g. a chloro, hydroxy, methyl or methoxy group. The 7-position is preferably unsubstituted. The ring carbon at the 8-position may if desired be replaced by a ring nitrogen and the ring nitrogen at the 7-position may if desired be replaced by a carbon.

In one preferred embodiment, the 2-position of the purine ring system is substituted by a nitro group or one of the substituents listed herein for the other ring positions, preferably a substituent other than chlorine, e.g. other than halogen. Especially preferably the 2-position substituted is carbon, nitrogen, oxygen or sulphur attached.

In another preferred embodiment, the 8-position of the purine ring system is substituted, e.g. by a hydrocarbyl, hydrocarbyloxy, hydroxy or halo group.

In yet another preferred embodiment, the 6-position of the purine ring system is occupied by a substituted 2-furyl group.

The compounds preferably fall into one, any two or all three of these three embodiments.

Especially preferably, the 8-position is unsubstituted, the 6-position carries a 2-furyl group, the 9-position carries a 4-methoxy-benzyl group, and the 2-position carries a small group containing 1 to 4 non hydrogen atoms, e.g. Cl, F, CH₃O, CH₃, C₂H₅O, CF₃, etc.

7-Deaza-purines in general, and their use form further aspects of the invention.

The compounds of the invention may if desired be presented in prodrug form, e.g. linked via a metabolically cleavable group, for example an ester group, to a biodistribution modifying group, for example a protein binding group such as for example an oligopeptide, a polysaccharide or a carboxylic acid. Such prodrug forms and their use are considered to fall within the scope of the invention.

Certain of the purine compounds according to the invention may be presented in salt form. In this event, the counterion is preferably one of those known to be physiologically tolerable, e.g. halide, sodium, mesylate, ammonium, meglumine, etc. The range of physiologically tolerable counterions is well known in the field of pharmacology.

The compounds of the invention may be synthesised by N-benzylation, e.g. using a benzyl chloride, of a 6-substituted purine, e.g. a 6-halopurine, followed, if required by palladium-catalysed coupling with an organometallic agent to introduce a 6-aryl group. Alternatively a 5-amino-4,6-dihalopyridine may be reacted with an aralkylamine and the product may be subjected to a ring closing reaction to create the purine ring system, whereafter the palladium catalysed coupling may be used to introduce a 6-aryl group. Following construction of the 9-benzyl compound, the substitution pattern on the benzyl group may be altered, e.g. by introduction or removal of hydroxy, thio or amino protecting groups. The 8-aza-purines of the invention may be prepared by ring closure of 7-chloro-5-amino-4-benzylamino-pyrimidines using sodium nitrite followed by introduction of the 6-aryl group in place of the chlorine analogously to the standard purines. The 7-deaza-purines of the invention may be prepared by N-benzylation of 4-chloro-7-deaza-purines, again followed by introduction of the 6-aryl group in place of the chlorine analogously to the standard purines. The synthesis routes are described further in J. Med. Chem. 48: 2710-2723 (2005) the contents of which are incorporated herein by reference.

The purine compounds of the invention may be formulated for administration using conventional carriers and excipients, e.g. water for injection, fillers, binders, pH modifiers, viscosity modifiers, flavours, etc. The formulations may be in any desired format suitable for the selected administration route, e.g. solutions, suspensions, dispersions, syrups, powders, sprays, tablets, coated tablets, capsules, suppositories, etc. Administration may be by any convenient route, e.g. oral, rectal, by injection, etc, although oral administration and intravenous injection are preferred.

The doses used will typically be in the range of 1 to 1000 mg per day for an adult human, although the precise dose to be used may be determined by routine dosage ranging experimentation, e.g. initially using animal models.

The invention will now be described further with reference to the following non-limiting Examples. Eluant ratios are by volume.

### EXAMPLE 1

### 6-Chloro-9-(3,4-dichlorophenylmethyl)-9H-purine

A mixture of 6-chloropurine (5.0 mmol) and potassium carbonate (2.07 g, 15 mmol) in dimethylformamide (20 mL) was stirred at ambient temperature under a nitrogen atmosphere for 30 minutes before 4-chlorobenzylchloride (10 mmol) was added. After stirring for 20 hours, the reaction mixture was filtered and evaporated in vacuo. The title product was isolated using flash chromatography on silica gel with EtOAc-hexane (1:1) as the eluant. The 9-benzylated isomer eluted first. Yield 953 mg (68%), mp 130-133° C., colorless small needles. ¹NMR (CDCl₃, 200 MHz) δ 5.41 (s, 2H, CH₂), 7.24 (d, J=8.0 Hz, 2H, Ar), 7.33 (d, J=8.0 Hz, 2H, Ar), 7.99 (s, 1H, H-8), 8.76 (s, 1H, H-2).

### EXAMPLE 2

### 6-Chloro-9-(3,4-dichlorophenylmethyl)-9H-purine

The title product was produced analogously to that of Example 1 using 3,4-dichlorobenzyl chloride. Yield 1.039 g (66%), mp 150-153° C., off-white powder. ¹H NMR (CDCl₃, 200 MHz) δ 5.39 (s, 2H, CH₂), 7.13 (dd, J=8.0 and 2.0 Hz, 1H, Ar), 7.40 (br s, 1H, Ar), 7.42 (d, J=8.0 Hz, 1H, Ar), 8.10 (s, 1H, H-8), 8.76 (s, 1H, H-2).

### EXAMPLE 3

### 6-Chloro-9-(4-fluorophenylmethyl)-9H -purine

The title product was produced analogously to that of Example 1 using 4-fluorobenzyl chloride. Yield 672 mg (51%), mp 129-131° C., colorless needles. ¹H NMR (CDCl₃, 200 MHz) δ 5.41 (s, 2H, CH₂), 7.00-7.08 (m, 2H, Ar), 7.27-7.34 (m, 2H, Ar), 8.07 (s, 1H, H-8), 8.77 (s, 1H, H-2).

### EXAMPLE 4

### 6-Chloro-9-(4-trifluoromethylphenylmethyl)-9H-purine

The title product was produced analogously to that of Example 1 using 4-trifluoromethylbenzyl chloride and using EtOAc-hexane (3:4) as the eluant. Yield 932 mg (60%), mp 130-132° C., colorless crystals. ¹H NMR (CDCl₃, 200 MHz) δ 5.51 (s, 2H, CH₂), 7.41 (dd, J=8.1 Hz, 2H, Ar), 7.63 (d, J=8.1 Hz, 2H, Ar), 8.12 (s, 1H, H-8), 8.77 (s, 1H, H-2); HRMS: Found 312.0379, calcd for C₁₃H₈F₃ClN₄ 312.0390; Anal. (C₁₃H₈F₃ClN₄) C, H, N.

### EXAMPLE 5

### 6-Chloro-9-(4-methylphenylmethyl)-9H-purine

The title product was produced analogously to that of Example 1 using 4-methylbenzyl chloride and using EtOAc-hexane (3:4) as the eluant. Yield 648 mg (50%), mp 135-136° C., colorless small needles. ¹NMR (CDCl₃, 200 MHz) δ 2.32 (s, 3H, CH₃), 5.39 (s, 2H, CH₂), 7.17 (m, 4H, Ar), 8.05 (s, 1H, H-8), 8.76 (s, 1H, H-2).

### EXAMPLE 6

### 9-(4-tert-Butylphenylmethyl)-6-chloro-9H-purine

The title product was produced analogously to that of Example 1 using 4-tert-butylbenzyl chloride and using EtOAc-hexane (1:2) and then EtOAc-hexane (1:1) as the eluant. Yield 636 mg (42%), off-white wax. ¹H NMR (CDCl₃, 200 MHz) δ 1.28 (s, 9H, t-Bu), 5.40 (s, 2H, CH₂), 7.23 (dd, J=8.4 Hz, 2H, Ar), 7.37 (d, J=8.4 Hz, 2H, Ar), 8.07 (s, 1H, H-8), 8.77 (s, 1H, H-2); HRMS: Found 300.1127, calcd for C₁₆H₁₇ClN₄ 300.1142; Anal. (C₁₆H₁₇ClN₄) C, H, N.

### EXAMPLE 7

### 6-Chloro-9-(4-methoxyphenylmethyl)-9H-purine

The title product was produced analogously to that of Example 1 using 4-methoxybenzyl chloride. Yield 611 mg (44%), mp 127-128° C., colorless crystals. ¹NMR (CDCl₃, 200 MHz) δ 3.78 (s, 3H, CH₃), 5.36 (s, 2H, CH₂), 6.87 (d, J=8.6 Hz, 2H, Ar), 7.26 (d, J=8.6 Hz, 2H, Ar), 8.04 (s, 1H, H-8), 8.77 (s, 1H, H-2).

### EXAMPLE 8

### 6-Chloro-9-(3-methoxyphenylmethyl)-9H-purine

The title product was produced analogously to that of Example 1 using 3-methoxybenzyl chloride. Yield 793 mg (58%), mp 104-105° C., colorless crystals. ¹H NMR (CDCl₃, 200 MHz) δ 3.76 (s, 3H, CH₃), 5.40 (s, 2H, CH₂), 6.83-6.87 (m, 3H, Ar), 7.23-7.31 (m, 1H, Ar), 8.07 (s, 1H, H-8), 8.76 (s, 1H, B-2)

### EXAMPLE 9

### 6-Chloro-9-(4-methoxy-3-methylphenylmethyl)-9H-purine

The title product was produced analogously to that of Example 1 using 4-methoxy-3-methylbenzyl chloride. Yield 505 mg (35%), colorless wax. ¹H NMR (CDCl₃, 200 MHz) δ 2.16 (s, 3H, CH₃), 3.80 (s, 3H, OCH₃), 5.33 (s, 2H, CH₂), 6.77 (d, J=8.0 Hz, 1H, Ar), 7.08-7.14 (m, 2H, Ar), 8.04 (s, 1H, H-8), 8.77 (s, 1H, H-2); HRMS: Found 288.0769, calcd for C₁₄H₁₃ClN₄O 288.0778; Anal. (C₁₄H₁₃ClN₄O) C, H, N, calcd, 19.41; found, 18.94.

### EXAMPLE 10

### 6-Chloro-9-(3,4,5-trimethoxyphenylmethyl)-9H-purine

The title product was produced analogously to that of Example 1 using 3,4,5-trimethoxybenzyl chloride and using EtOAc-hexane (2:1) as the eluant. Yield 850 mg (51%), mp 115-116° C., colorless crystals. ¹NMR (CDCl₃, 300 MHz) δ 3.75 (s, 9H, 3×OCH₃), 5.31 (s, 2H, CH₂), 6.49 (s, 2H, Ar), 8.07 (s, 1H, H-8), 8.72 (s, 1H, H-2); HRMS: Found 344.0835, calcd for C₁₅H₁₅N₄O₃Cl 334.0832; Anal. (C₁₅H₁₅N₄O₃Cl) C, H, N.

### EXAMPLE 11

### 6-Chloro-9-(4-trifluoromethoxyphenylmethyl)-9H-purine

The title product was produced analogously to that of Example 1 on a 2.0 mmol scale using 4-trifluoromethoxybenzyl chloride and using EtOAc-hexane (2:3) as the eluant. Yield 338 mg (51%), mp 115-116° C., colorless crystals. ¹H NMR (CDCl₃, 200 MHz) 5.45 (s, 2H, CH₂), 7.18-7.27 (m, 2H, Ar), 7.33-7.37 (m, 2H, Ar), 8.10 (s, 1H, H-8), 8.77 (s, 1H, H-2); HRMS: Found 328.0330, calcd for C₁₃H₁₈ClF₂N₄O 328.0339; Anal. (C₁₃H₁₈ClF₃N₄O) C, H, N.

### EXAMPLE 12

### 6-Chloro-9-(3-trifluoromethoxyphenylmethyl)-9H-purine

The title product was produced analogously to that of Example 1 on a 2.2 mmol scale using 3-trifluoromethoxybenzyl chloride and using acetone-hexane (2:5) as the eluant. Yield 343 mg (47%), colorless oil. ¹H NMR (CDCl₃, 200 MHz) 5.46 (s, 2H, CH₂), 7.18-7.25 (m, 3H, Ar), 7.35-7.43 (m, 1H, Ar), 8.11 (s, 1H, H-8), 8.77 (a, 1H, H-2); HRMS: Found 328.0335, calcd for C₁₃H₁₈ClF₃N₄O 328.0339.

### EXAMPLE 13

### 6-Chloro-9-(4-ethoxyphenymethyl)-9H-purine

The title product was produced analogously to that of Example 1 using 4-ethoxybenzyl chloride. Yield 606 mg (42%), mp 110-112° C., colorless crystals. ¹H NMR (CDCl₃, 200 MHz) δ 1.38 (t, J=7.2 Hz, 3H, CH₃), 3.99 (q, J=7.2 Hz, 2H, OCH₂), 5.35 (s, 2H, CH₂), 6.85 (d, J=8.8 Hz, 2H, Ar), 7.25 (d, J=8.8 Hz, 2H, Ar), 8.04 (s, 1H, H-8), 8.76 (s, 1H, H-2); HRMS: Found 288.0777, calcd for C₁₄H₁₃ClN₄O 288.0778; Anal. (C₁₄H₁₃ClN₄O) C, H, N, calcd, 19.41; found, 18.95.

### EXAMPLE 14

### 9-(4-Benzyloxyphenylmethyl)-6-chloro-9H-purine

The title product was produced analogously to that of Example 1 using 4-benzyloxybenzyl chloride and using EtOAc-hexane (2:1) as the eluant. Yield 1.140 g (65%), mp 127-128° C., colorless crystals. ¹H NMR (CDCl₃, 300 MHz) δ 4.90 (s, 2H, CH₂), 5.22 (s, 2H, CH₂), 6.79-7.51 (m, 9H, Ar), 7.91 (s, 1H, H-8), 8.63 (s, 1H, H-2).

### EXAMPLE 15

### 6-Chloro-9-(4-methylthiophenylmethyl)-9H-purine

The title product was produced analogously to that of Example 1 on a 2.5 mmol scale using 4-methylthiobenzyl chloride. Yield 346 mg (49%), mp 95-97° C., colorless needles. ¹H NMR (CDCl₃, 200 MHz) δ 2.45 (s, 3H, CH₃), 5.39 (s, 2H, CH₂), 7.22 (s, 4H, Ar), 8.07 (s, 1H, H-8), 8.77 (s, 1H, H-2). HRMS: Found 290.0400, calcd for C₁₃H₁₁ClN₄S 290.0393; Anal. (C₁₃H₁₁ClN₄S) C, H, N.

### EXAMPLE 16

### 9-(4-Acetamidophenylmethyl)-6-chloro-9H-purine

The title product was produced analogously to that of Example 1 on a 2.3 mmol scale using 4-acetamidobenzyl chloride and using acetane-hexane (2:3) as the eluant. Yield 431 mg (62%), mp 240-242° C., colorless small needles. ¹H NMR (CDCl₃, 200 MHz) δ 2.15 (s, 3H, CH₃), 5.39 (s, 2H, CH₂), 7.19 (br s, 1H, NH), 7.28 (d, J=8.0 Hz, 2H, Ar), 7.50 (d, J=8.0 Hz, 2H, Ar), 8.06 (s, 1H, H-8), 8.76 (s, 1H, H-2). HRMS: Found 301.0724, calcd for C₁₄H₁₂ClN₅O 301.0730; Anal. (C₁₄H₁₂ClN₆O) C, H.

### EXAMPLE 17

### 9-(4-Chlorophenylmethyl)-2,6-dichloro-9H-purine

The title product was produced analogously to that of Example 1 using 2,6-dichloropurine and 4-chlorobenzyl chloride and using EtOAc-hexane (2:3) as the eluant. Yield 856 mg (55%), mp 162-164° C., colorless small needles. ¹H NMR (CDCl₃, 200 MHz) δ 5.36 (s, 2H, CH₂), 7.21-7.26 (m, 2H, Ar), 7.33-7.37 (m, 2H, Ar), 8.04 (s, 1H, H-8).

### EXAMPLE 18

### 2,6-Dichloro-9-(3,4-dichlorophenylmethyl)-9H-purine

The title product was produced analogously to that of Example 1 using 2,6-dichloropurine and 3,4-dichlorobenzyl chloride and using EtOAc-hexane (1:2) and then EtOAc-hexane (1:1) as the eluant. Yield 962 mg (56%), mp 175-177° C., colorless crystals. ¹H NMR (CDCl₂, 200 MHz) δ 5.35 (s, 2H, CH₂), 7.14 (dd, J=8.2 and 2.2 Hz, 1H, Ar), 7.39 (d, J=2.2 Hz, 1H, Ar), 7.44 (d, J=8.2 Hz, 1H, Ar), 8.06 (s, 1H, M-8), HRMS: Found 345.9351, calcd for C₁₂H₆Cl₄N₄ 345.9347; Anal. (C₁₂H₆Cl₄N₄) C, N, H, calcd, 1.16; found, 1.81.

### EXAMPLE 19

### 9-(3-Chlorophenylmethyl)-2,6-dichloro-9H-purine

The title product was produced analogously to that of Example 1 using 2,6-dichloropurine and 3-chlorobenzyl chloride and using EtOAc-hexane (1:3) as the eluant. Yield 871 mg (56%), mp 122-125° C., colorless crystals. ¹H NMR (CDCl₃, 200 MHz) δ 5.36 (s, 2H, CH₂), 7.18-7.32 (m, 4H, Ar), 8.05 (s, 1H, H-8); ¹³C NMR (CDCl₃, 50 MHz) δ 47.3 (CH₂), 126.0 (CH in Ar), 128.0 (CH in Ar), 129.2 (CH in Ar), 130.2 (C-5), 130.6 (CH in Ar), 135.2 (C in Ar), 135.9 (C in Ar), 145.2 (C-8), 151.9 (C-2/C-4/C-6), 153.0 (C-2/C-4/C-6), 153.2 (C-2/C-4/C-6); MS EI m/z (rel. %): 316/314/312 (12/39/41, M⁺), 279 (11), 277 (18), 201 (6), 127 (31), 125 (100), 99 (7), 89 (21); HRMS: Found 311.9737, calcd for C₁₂H₇Cl₃N₄ 311.9736; Anal. (C₁₂H₇Cl₂N₄) C, H, N.

### EXAMPLE 20

### 2,6-Dichloro-9-(4-fluorophenylmethyl)-9H-purine

The title product was produced analogously to that of Example 1 using 2,6-dichloropurine and 4-fluorobenzyl chloride and using EtOAc-hexane (1:3) as the eluant. Yield 817 mg (55%), mp 120-122° C., colorless crystals. ¹H NMR (CDCl₃, 200 MHz) δ 5.36 (s, 2H, CH₂), 7.01-7.10 (m, 2H, Ar), 7.27-7.34 (m, 2H, Ar), 8.02 (s, 1H, H-8); HRMS: Found 296.0019, calcd for C₁₂H₇Cl₂FN₄ 296.0032; Anal. (C₁₂H₇Cl₂FN₄) C, H, N.

### EXAMPLE 21

### 2,6-Dichloro-9-(4-methylphenylmethyl)-9H-purine

The title product was produced analogously to that of Example 1 using 2,6-dichloropurine and 4-methylbenzyl chloride and using EtOAc-hexane (1:2) as the eluant. Yield 772 mg (53%), mp 138-141° C., colorless micro-crystalline solid. ¹H NMR (CDCl₃, 200 MHz) δ 2.33 (s, 3H, CH₃), 5.34 (s, 2H, CH₂), 7.18 (m, 4H, Ar), 8.00 (s, 1H, H-8).

### EXAMPLE 22

### 2,6-Dichloro-9-(4-methoxyphenylmethyl)-9H-purine

The title product was produced analogously to that of Example 1 using 2,6-dichloropurine and 4-methoxybenzyl chloride and using EtOAc-hexane (2:3) as the eluant. Yield 837 mg (54%), mp 128-130° C., colorless crystals. ¹H NMR (CDCl₃, 200 MHz) δ 3.79 (s, 3H, CH₃), 5.32 (s, 2H, CH₃), 6.88 (d, J=8.2 Hz, 2H, Ar), 7.26 (d, J=8.2 Hz, 2H, Ar), 7.99 (s, 1H, H-8).

### EXAMPLE 23

### 2,6-Dichloro-9-(3-methoxyphenylmethyl)-9H-purine

The title product was produced analogously to that of Example 1 using 2,6-dichloropurine and 3-methoxybenzyl chloride and using EtOAc-hexane (1:2) as the eluant. Yield 835 mg (54%), mp 119-121° C., colorless small needles. ¹H NMR (CDCl₃, 200 MHz) δ 3.79 (s, 3H, CH₃), 5.35 (s, 2H, CH₂), 6.83-6.89 (m, 3H, Ar), 7.28 (t, J=8.2 Hz, 1H, Ar), 8.02 (H-8): Found 308.0238, calcd for C₁₃H₁₀Cl₂N₄O 308.0232; Anal. (C₁₃H₁₀Cl₂N₄O) C, H, N.

### EXAMPLE 24

### 2,6-Dichloro-9-(4-methoxy-3-methylphenylmethyl)-9H-purine

The title product was produced analogously to that of Example 1 using 2,6-dichloropurine and 4-methoxy-3-methylbenzyl chloride and using EtOAc-hexane (1:2) as the eluant. Yield 625 mg (39%), mp 134-135° C., colorless powdery crystals. ¹H NMR (CDCl₂, 200 MHz) δ 2.18 (s, 3H, CH₃), 3.81 (s, 3H, OCH₃), 5.28 (s, 2H, CH₂), 6.79 (d, J=8.2 Hz, 1H, Ar), 7.08-7.16 (m, 2H, Ar), 7.99 (s, 1H, H-8); HRMS: Found 322.0382, calcd for C₁₄H₁₂Cl₂N₄O 322.0388; Anal. (C₁₄H₁₂Cl₂N₄O) C, H, N.

### EXAMPLE 25

### 6-Chloro-9-(4-dimethylaminophenylmethyl)-9H-purine

Ethanesulfonic acid (0.010 mL, 0.12 mmol) was added toga stirred suspension of 5-amino-4-chloro-6-(4-di-methylaminophenyl)pyrimidine (316 mg, 1.14 mmol) in triethyl orthoformate (15 mL). The resulting mixture was stirred for 3 days and evaporated in vacuo. The residue was dissolved in dichloromethane (50 mL) and washed with saturated aqueous NaHCO₃ (10 mL) and brine (2×10 mL), dried (MgSO₄) and evaporated in vacuo. The crude product was purified by flash chromatography on silica gel eluting with acetone-hexane (2:5). Yield 215 mg (66%), mp 125-127° C., colorless crystals. ¹H NMR (CDCl₃, 200 MHz) δ 2.94 (s, 6H, CH₃), 5.32 (s, 2H, CH₂), 6.68 (d, J=8.6 Hz, 2H, Ar), 7.22 (d, J=8.6 Hz, 2H, Ar), 8.04 (s, 1H, H-8), 8.77 (s, 1H, H-2); HRMS: Found 287.0932, calcd for C₁₄H₁₄ClN₅ 287.0938.

### EXAMPLE 26

### 9-(4-Acetoxyphenylmethyl)-6-chloro-9H-purine

The title product was produced analogously to that of Example 1 using 4-acetoxybenzyl chloride and using EtOAc-hexane (1:1) as eluant. Yield 387 mg (51%), mp 120-122° C., colorless crystals. ₁H NMR (CDCl₃, 200 MHz) δ 2.27 (s, 3H, CH₂), 5.43 (s, 2H, CH₂), 7.08 (d, J=8.6 Hz, 2H, Ar), 7.23 (d, J=8.6 Hz, 2H, Ar), 8.09 (s, 1H, H-8), 8.77 (s, 1H, H-2); HRMS: Found 302.0575, calcd for C₁₄H₁₁ClN₄O₂ 302.0571, Anal. (C₁₄H₁₁ClN₄O₂) C, H, N.

### EXAMPLE 27

### 9-(4-Chlorophenylmethyl)-6-(2-furyl)-9H-purine

A mixture of the compound of Example 1 (1.0 mmol), bi(triphenylphosphine)palladium (II) chloride (35 mg, 0.05 mmol) and 2-furyl(tributyl) tin (0.47 mL, 1.4 mmol) in DMF (5 EL) was stirred at 90° C. under a nitrogen atmosphere for 18 hours and evaporated in vacuo. A saturated solution of potassium fluoride in methanol was added to the residue and the mixture was stirred overnight and then evaporated in vacuo with a small amount of silica gel. The residue was added on the top of a chromatography column, and the title product was isolated by flash chromatography on silica using EtOAc-hexane (1:1) and then EtOAc-hexane (2;1) as the eluant. Yield 239 mg (77%), mp 156-159° C., colorless crystals. ¹H NMR (CDCl₃, 200 MHz) δ 5.42 (s, 2H, CH₂), 6.65 (dd, J=3.6 and 1.8 Hz, 1H, H-4 in furyl), 7.23 (d, J=8.6 Hz, 2H, Ar), 7.32 (d, J=8.6 Hz, 2H, Ar), 7.75 (br d, J=1.8 Hz, 1H, H-5 in furyl), 7.83 (br d, J=3.6 Hz, 1H, H-3 in furyl), 8.05 (s, 1H, H-8), 9.06 (s, 1H, H-2); ¹³C NMR (CDCl₃, 50 MHz) δ 46.5 (CH₂), 112.6 (C-4 in furyl), 117.4 (C-3 in furyl), 129.1 (CH in Ar), 129.3. (CH in Ar), 129.6 (C-5), 133.6 (C in Ar), 134.6 (C in Ar), 144.0 (C-8), 145.9 (C-5 in furyl), 146.0 (C-6), 149.6 (C-2 in furyl), 152.0 (C-4), 152.8 (C-2); MS EI m/z (rel. 4): 312/310 (37/100, M⁺), 281 (11), 199 (10), 138 (7), 127 (26), 126 (6), 125 (71), 199 (4), 90 (5), 89 (16); HRMS: Found 310.0625, calcd for C₁₆H₁₁ClN₄O 310.0621; Anal. (C₁₆H₁₀ClN₄O) C, H, N.

### EXAMPLE 28

### 9-(3,4-Dichlorophenylmethyl)-6-(2-furyl)-9H-purine

The title product was produced analogously to that of Example 27 using the compound of Example 2 and using EtOAc-hexane (1:1) and then EtOAc-hexane (2:1) as the eluant. Yield 282 mg (82%), mp 183-185° C., colorless microcrystalline solid. ¹H NMR (CDCl₃, 200 MHx) δ 5.40 (s, 2H, CH₂), 6.66 (dd, J=3.4 and 1.8 Hz, 1H, H-4 in furyl), 7.13 (dd, J=8.4 and 1.8 Hz, 1H, Ar), 7.39 (br s, 1H, Ar), 7.42 (d, J=8.4 Hz, 1H, Ar), 7.76 (dd, J=1.8 and 0.8 Hz, 1H, H-5 in furyl), 7.84 (dd, J=3.4 and 1.8 Hz, 1H, H-3 in furyl), 8.07 (s, 1H, H-8), 8.96 (s, 1H, H-2); Anal. (C₁₆H₁₀Cl₂N₄O) C, H, N.

### EXAMPLE 29

### 9-(4-Fluorophenylmethyl)-6-(2-furyl)-9H-purine

The title product was produced analogously to that of Example 27 using the compound of Example 3 and using EtOAc-hexane (2:1) as the eluant. Yield 269 mg (91%), mp 162-164° C., colorless crystals. ¹H NMR (CDCl₃, 200 MHz) δ 5.42 (s, 2H, CH₂), 6.64 (dd, J=3.4 and 1.8 Hz, 1H, H-4 in furyl), 7.03-7.12 (m, 2H, Ar), 7.32-7.38 (m, 2H, Ar), 7.75 (m, 1H, H-5 in furyl), 7.82 (dd, J=3.4 and 0.6 Hz, 1H, H-3 in furyl), 8.13 (s, 1H, H-8), 8.98 (s, 1H, H-2); HRMS: Found 294.0889, calcd for C₁₆H₁₁FN₄O 294.0917; Anal. (C₁₆H₁₁FN₄O) C, H, N.

### EXAMPLE 30

### 6-(2-Furyl)-9-(4-trifluoromethylphenylmethyl)-9H-purine

The title product was produced analogously to that of Example 27 using the compound of Example 4 and using EtOAc-hexane (2:1) and then pure EtOAc as the eluant. Yield 328 mg (95%), mp 159-161° C., off-white crystals. ¹H NMR (CDCl₃, 200 MHz) δ 5.52 (s, 2H, CH₂), 6.66 (dd, J=3.4 and 1.6 Hz, 1H, H-4 in furyl), 7.40 (d, J=8.2 Hz, 2H, Ar), 7.60 (d, J=8.2 Hz, 2H, Ar), 7.76 (m, 1H, H-5 in furyl), 7.84 (br d, J=3.4, 1H, H-3 in furyl), 8.07 (s, 1H, H-8), 8.96 (s, 1H, H-2); HRMS: Found 344.0871, calcd for C₁₇H₁₁F₃N₄O 344.0885; Anal. (C₁₇H₁₁F₃N₄O) C, H, N.

### EXAMPLE 31

### 6-(2-Furyl)-9-(4-methylphenylmethyl)-9H-purine

The title product was produced analogously to that of Example 27 using the compound of Example 5. Yield 245 mg (84%), mp 166-167° C., off-white powdery crystals. ¹H NMR (CDCl₃, 200 MHz) δ 2.31 (s, 3H, CH₃), 5.40 (s, 2H, CH₂), 6.64 (dd, J=3.4 and 1.8 Hz, 1H, H-4 in furyl), 7.14 (d, J=8.4 Hz, 2H, Ar), 7.20 (d, J=8.4 Hz, 2H, Ar), 7.75 (m, 1H, H-5 in furyl), 7.82 (br d, J=3.4 Hz, 1H, H-3 in furyl), 8.04 (s, 1H, H-8), 8.96 (s, 1H, H-2); HRMS: Found 290.1167, calcd for C₁₇H₁₄N₄O 290.1168; Anal. (C₁₇H₁₄N₄O) C, H, N.

### EXAMPLE 32

### 9-(4-tert-Butylphenylmethyl)-6-(2-furyl)-9H-purine

The title product was produced analogously to that of Example 27 using the compound of Example 6 and using EtOAc-hexane (1:1) as the eluant. Yield 263 mg (79%), mp 185-187° C., off-white crystals. ¹H NMR (CDCl₃, 200 MHz) δ 1.28 (s, 9H, t-Bu), 5.42 (s, 2H, CH₂), 6.65 (dd, J=3.4 and 1.6 Hz, 1H, H-4 in furyl), 7.23 (d, J=8.4 Hz, 2H, Ar), 7.37 (d, J=8.4 Hz, 2H, Ar), 7.75 (m, 1H, H-5 in furyl), 7.83 (br d, J=3.4 Hz, 1H, H-3 in furyl), 8.06 (s, 1H, H-8), 8.97 (s, 1H, H-2); HRMS: Found 322.1631, calcd for C₂₀H₂₀N₄O 322.1637; Anal. (C₂₀H₂₀N₄O) C, H, N.

### EXAMPLE 33

### 6-(2-Furyl)-9-(4-methoxyphenylmethyl)-9H-purine

The title product was produced analogously to that of Example 27 using the compound of Example 7. Yield 237 mg (77%), mp 175-176° C., colorless crystals. ¹H NMR (CDCl₂, 200 MHz) δ 3.77 (s, 3H, CH₃), 5.37 (s, 2H, CH₂), 6.64 (dd, J=3.4 and 1.6 Hz, 1H, H-4 in furyl), 6.86 (d, J=8.0 Hz, 2H, Ar), 7.26 (d, J=8.4 Hz, 2H, Ar), 7.75 (m, 1H, H-5 in furyl), 7.81 (br d, J=3.4 Hz, 1H, H-3 in furyl), 8.02 (s, 1H, H-8), 8.96 (s, 1H, H-2); HRMS: Found 306.1130, calcd for C₁₇H₁₄N₄O₂ 306.1117; Anal. (C₁₇H₁₄N₄O₂) C, H, N.

### EXAMPLE 34

### 6-(2-Furyl)-9-(3-methoxyphenylmethyl)-9H-purine

The title product was produced analogously to that of Example 27 using the compound of Example 8 on a 2 mmol scale. Yield 575 mg (94%), mp 138-139° C., colorless crystals. ¹H NMR (CDCl₃, 200 MHz) δ 3.75 (s, 3H, CH₃), 5.41 (s, 2H, CH₂), 6.65 (dd, J=3.4 and 1.8 Hz, 1H, H-4 in furyl), 6.83-6.87 (m, 3H, Ar), 7.21-7.30 (m, 1H, Ar), 7.75 (m, 1H, H-5 in furyl), 7.83 (dd, J=3.4 and 0.6 Hz, 1H, H-3 in furyl), 8.06 (s, 1H, H-8), 8.97 (s, 1H, H-2); HRMS: Found 306.1112, calcd for C₁₇H₁₄N₄O₂ 306.1117; Anal. (C₁₇H₁₄N₄O₂) C, H, N.

### EXAMPLE 35

### 6-(2-Furyl)-9-(4-methoxy-3-methylphenylmethyl)-9H-purine

The title product was produced analogously to that of Example 27 using the compound of Example 9 and using EtOAc-hexane (2:1) as the eluant. Yield 285 mg (89%), mp 169-170° C., colorless crystals. ¹H NMR (CDCl₃, 200 MHz) δ 2.16 (s, 3H, CH₃), 3.79 (s, 3H, OCH₃), 5.34 (s, 2H, CH₂), 6.64 (dd, J=3.6 and 1.6 Hz, 1H, H-4 in furyl), 6.77 (d, J=8.4 Hz, 1H, Ar), 7.08-7.15 (m, 2H, Ar), 7.75 (m, 1H, furyl), 7.82 (m, 1H, furyl), 8.02 (s, 1H, H-8), 8.87 (s, 1H, H-2); HRMS: Found 320.1288, calcd for C₁₈H₁₆N₄O₂ 320.1273; Anal. (C₁₈H₁₆N₄O₂) C, H, N.

### EXAMPLE 36

### 6-(2-Furyl)-9-(3,4,5-trimethoxyphenylmethyl)-9H-purine

The title product was produced analogously to that of Example 27 using the compound of Example 10 and using EtOAc-hexane (3:1) as the eluant. Yield 246 mg (67%), mp 195-196° C., colorless crystals. ¹H NMR (CDCl₃, 300 MHz) δ 3.78 (s, 6H, 2×CH₃), 3.79 (s, 3H, CH₂), 5.35 (s, 2H, CH₂), 6.52 (s, 2H, Ar), 6.65 (dd, J=3.6 and 1.6 Hz, 1H, H-4 in furyl), 7.74 (m, 1H, furyl), 8.06 (s, 1H, H-8), 8.96 (s, 1H, H-2); HRMS: Found 366.1323, calcd for C₁₉H₁₈N₄O₄ 366.1328; Anal. (C₁₉H₁₈N₄O₄) C, H, N.

### EXAMPLE 37

### 6-(2-Furyl)-9-(4-trifluoromethoxyphenylmethyl)-9H-purine

The title product was produced analogously to that of Example 27 using the compound of Example 11 on a 0.5 mmol scale. Yield 158 mg (88%), mp 146-147° C., off-white crystals. ¹H NMR (CDCl₃, 200 MHz) δ 5.46 (s, 2H, CH₂), 6.66 (dd, J=3.6 and 1.6 Hz, 1H, H-4 in furyl), 7.19 (d, J 8.6 Hz, 2H, Ar), 7.34 (d, J=8.6 Hz, 2H, Ar), 7.76 (m, 1H, H-5 in furyl), 7.84 (br d, J=3.6 Hz, 1H, H-3 in furyl), 8.08 (s, 1H, H-8), 8.97 (s, 1H, H-2); HRMS: Found 360.0839, calcd for C₁₇H₁₁F₃N₄O₂ 360.0834; Anal. (C₁₇H₁₁F₃N₄O₂) C, H, N.

### EXAMPLE 38

### 6-(2-Furyl)-9-(3-trifluoromethoxyphenylmethyl)-9H-purine

The title product was produced analogously to that of Example 27 using the compound of Example 12 on a 0.28 mmol scale and using acetone-hexane (2:5) as the eluant. Yield 72 mg (71%), mp 133-135° C., colorless crystals. ¹H NMR (CDCl₃, 200 MHz) δ 5.48 (s, 2H, CH₂), 6.66 (dd, J=3.6 and 1.6 Hz, 1H, H-4 in furyl), 7.18-7.24 (m, 3H, Ar), 7.34-7.38 (m, 1H, Ar), 7.77 (m, 1H, H-5 in furyl), 7.87 (br d, J=3.6 Hz, 1H, H-3 in furyl), 8.09 (s, 1H, H-8), 8.98 (s, 1H, H-2); HRMS: Found 360.0840, calcd for C₁₇H₁₁F₃N₄O₂ 360.0834; Anal. (C₁₇H₁₁F₃N₄O₂) C, H, N.

### EXAMPLE 39

### 9-(4-Ethoxyphenylmethyl)-6-(2-furyl)-9H-purine

The title product was produced analogously to that of Example 27 using the compound of Example 13. Yield 277 mg (87%), mp 165-166° C., off-white crystals. ¹H NMR (CDCl₃, 200 MHz) δ 1.38 (t, J=7.0 Hz, 3H, CH₃), 4.00 (q, J=7.0 Hz, 2H, OCH₂), 5.37 (s, 2H, CH₂), 6.65 (dd, J 3.6 and 1.6 Hz, 1H, H-4 in furyl), 6.86 (d, J=8.8 Hz, 2H, Ar), 7.22 (d, J=8.8 Hz, 2H, Ar), 7.74 (m, 1H, H-5 in furyl), 7.82 (br d, J=3.6 Hz, 1H, H-3 in furyl), 8.03 (s, 1H, H-8), 8.96 (s, 1H, H-2); HRMS: Found 320.1277, calcd for C₁₈H₁₆N₄O₂ 320.1273; Anal. (C₁₉H₁₆N₄O₂) C, H, N.

### EXAMPLE 40

### 9-(4-Benzyloxyphenylmethyl)-6-(2-furyl)-9H-purine

The title product was produced analogously to that of Example 27 using the compound of Example 14 and using EtOAc-hexane (3:1) as the eluant. Yield 110 mg (71%), mp 145-146° C., colorless crystals. ¹H NMR (CDCl₃, 300 MHz); δ 5.03 (s, 2H, OCH₂), 5.37 (s, 2H, NCH₂), 6.65 (dd, J=3.4 and 1.6 Hz, 1H, H-4 in furyl), 6.92-6.95 (m, 2H, Ar), 7.26-7.39 (m, 7H, Ar), 7.75 (br s, 1H, H-5 in furyl), 7.81 (br d, J=3.4 Hz, 1H, H-3 in furyl), 8.03 (s, 1H, H-8), 8.96 (s, 1H, H-2); HRMS: Found 382.1424, calcd for C₂₃H₁₉N₄O₂ 382.1429; Anal. (C₂₃H₁₈N₄O₂) C, H, N.

### EXAMPLE 41

### 6-(2-Furyl)-9-(4-hydroxyphenylmethyl)-9H-purine

The title product was produced analogously to that of Example 27 using the compound of Example 26 and using EtOAc-hexane (2:1) and then EtOAc as the eluant. Yield 181 mg (56%), mp 231-233° C., colorless crystals. ¹H NMR (DMSO-d₆, 200 MHz) δ 5.37 (s, 2H, CH₂), 6.70 (br d, J=8.4 Hz, 2H, Ar), 6.79 (dd, J=3.6 and 1.8 Hz, 1H, H-4 in furyl), 7.21 (br d, J=8.4 Hz, 2H, Ar), 7.81 (m, 1H, furyl), 8.04 (m, 1H, furyl), 8.70 (s, 1H, H-8), 8.88 (s, 1H, H-2), 9.46 (s, 1H, OH); HRMS: Found 292.0972, calcd for C₁₆H₁₂N₄O₂ 292.0960, Anal. (C₁₆H₁₂N₄O₂) C, H, N.

### EXAMPLE 42

### 6-(2-Furyl)-9-(4-methylthiophenylmethyl)-9H-purine

The title product was produced analogously to that of Example 27 using the compound of Example 15 and using acetone-hexane (2:3) as the eluant. Yield 309 mg (96%), mp 157-159° C., colorless crystals. ¹H NMR (CDCl₃, 200 MHz) δ 2.50 (s, 3H, CH₃), 5.40 (s, 2H, CH₂), 6.65 (dd, J=3.6 and 1.6 Hz, 1H, H-4 in furyl), 7.22-7.24 (m, 4H, Ar), 7.75 (m, 1H, furyl), 7.82 (m, 1H, furyl), 8.05 (s, 1H, H-8), 8.96 (s, 1H, H-2); HRMS: Found 322.0898, calcd for C₁₇H₁₄N₄OS 322.0888; Anal. (C₁₇H₁₄N₄OS) C, H, N.

### EXAMPLE 43

### 9-(4-Dimethylaminophenylmethyl)-6-(2-furyl)-9H-purine

The title product was produced analogously to that of Example 27 using the compound of Example 25 on a 0.5 mmol scale and using acetone-hexane (2:5) as the eluant. Yield 140 mg (88%), mp 158-160° C., colorless crystals. ¹H NMR (CDCl₃, 200 MHz) δ 2.93 (s, 6H, CH₃), 5.33 (s, 2H, CH₂), 6.64 (dd, J=3.6 and 1.6 Hz, 1H, H-4 in furyl), 6.69 (d, J=8.6 Hz, 2H, Ar), 7.22 (d, J=8.6 Hz, 2H, Ar), 7.74 (dd, J=1.6 and 0.6 Hz, 1H, H-5 in furyl), 7.81 (dd, J=3.6 and 0.6 Hz, 1H, H-3 in furyl), 8.02 (s, 1H, H-8), 8.97 (s, 1H, H-2); HRMS: Found 319.1430, calcd for C₁₉H₁₇N₅O 319.1433; Anal. (C₁₈H₁₇N₅O) C, H, N, calcd, 21.93; found, 21.25.

### EXAMPLE 44

### 9-(4-Acetamidophenylmethyl)-6-(2-furyl)-9H-purine

The title product was produced analogously to that of Example 27 using the compound of Example 16 on a 1.1 mmol scale and using methanol:CHCl₃ (1:15) as the eluant. Yield 335 mg (88%), mp 278-280° C., colorless powdery crystals. ¹H NMR (DMSO-d₆, 200 MHz) δ 2.00 (s, 3H, CH₃), 5.44 (s, 2H, CH₂), 6.80 (dd, J=3.6 and 1.8 Hz, 1H, H-4 in furyl), 7.30 (d, J=8.4 Hz, 2H, Ar), 7.52 (d, J=8.4 Hz, 2H, Ar), 7.83 (br d, J=3.6 Hz, 1H, H-3 in furyl), 8.05 (m, 1H, H-5 in furyl), 8.72 (s, 1H, H-8), 8.88 (s, 1H, H-2), 9.94 (br s, 1H, NH); HRMS: Found 333.1229, calcd for C₁₉H₁₅N₅O₂ 333.1226; Anal. (C₁₈H₁₅N₅O₂) C, H, N.

### EXAMPLE 45

### 9-(4-Aminophenylmethyl)-6-(2-furyl)-9H-purine

A mixture of the compound of Example 44 (140 mg, 0.42 ML) in 48% aqueous HBr (2 mL) was stirred at 90° C. for 17 hours and cooled to ambient temperature, before 10% aqueous K₂CO₃ (10 mL) was added carefully and the pH was adjusted to ca. 11 by addition of solid K₂CO₃. The mixture was extracted with EtOAc (4×25 mL) and the combined organic layers were dried (MgSO₄) and evaporated in vacuo. The crude product was purified by flash chromatography on silica gel eluting with EtOAc. Yield 82 mg (70%), mp 195-197° C., yellow powdery crystals. ¹H NMR (DMSO-d₆, 200 MHz) δ 5.10 (br s, 2H, NH₂), 5.28 (s, 2H, CH₂), 6.49 (d, J=8.4 Hz, 2H, Ar), 6.79 (dd, J=3.4 and 1.6 Hz, 1H, H-4 in furyl), 7.08 (d, J=8.4 Hz, 2H, Ar), 7.81 (br d, J=3.4 Hz, 1H, H-3 in furyl), 8.04 (br s, 1H, H-5 in furyl), 8.66 (s, 1H, H-8), 8.88 (s, 1H, H-2); HRMS: Found 291.1125, calcd for C₁₆H₁₃N₅O 291.1120; Anal. (C₁₆H₁₃N₅O) C, H.

### EXAMPLE 46

### 2-Chloro-9-(4-chlorophenylmethyl)-6-(2-furyl)-9H-purine

A mixture of tris(dibenzylideneacetone)dipalladium chloroform adduct (16 mg, 0.015 mmol) and tri(2-furyl)phosphine (26 mg, 0.11 mmol) in DMF (4 mL) was stirred at ambient temperature under nitrogen atmosphere for 15 minutes whereafter the compound of Example 17 (0.5 mmol) and 2-furyl(tributyl) tin (0.20 mL, 0.6 mmol) were added. The resulting mixture was stirred for 8 hours at 50° C. and evaporated in vacuo. The title compound was extracted and purified analogously to Example 27. ¹³C NMR (CDCl₃, 50 MHz) δ 4.67 (CH₂), 112.8 (C-4 in furyl), 118.8 (C-3 in furyl), 128.0 (C-5), 129.2 (CH in Ar), 129.3 (CH in Ar), 133.1 (C in Ar), 134.7 (C in Ar), 144.5 (C-8), 146.7 (C-5 in furyl), 147.3 (C-6), 148.7 (C-2 in furyl), 153.5 (C-4), 156.0 (C-2); MS EI m/z (rel. %): 348/346/344 (6/53/84, M⁺), 343 (25), 315 (2), 309 (5), 233 (1), 127 (29), 126 (4), 125 (100), 99 (2), 90 (3).

### EXAMPLE 47

### 2-Chloro-9-(3,4-dichlorophenylmethyl)-6-(2-furyl)-9H-purine

The title compound was produced analogously to that of Example 46 using the compound of Example 18 and using EtOAc-hexane (1:2) and then EtOAc-hexane (1:1) as the eluant. Yield 142 mg (75%), mp 191-193° C., colorless crystals. ¹H NMR (CDCl₃, 200 MHz) δ 5.36 (s, 2H, CH₂), 6.66 (dd, J=3.4 and 1.8 Hz, 1H, H-4 in furyl), 7.13 (d, J=8.4 and 1.8 Hz, 1H, Ar), 7.38-7.46 (m, 2H, Ar), 7.78 (m, 1H, H-5 in furyl), 7.87 (br d, J=3.4 Hz, 1H, H-3 in furyl), 8.02 (s, 1H, H-8); HRMS: Found 377.9838, calcd for C₁₆H₁₉Cl₃N₄O 377.9842; Anal. (C₁₆H₁₉Cl₃N₄O) C, H, N, calcd, 14.76; found, 14.16.

### EXAMPLE 48

### 2-Chloro-9-(3-chlorophenylmethyl)-6-(2-furyl)-9H-purine

The title compound was produced analogously to that of Example 46 using the compound of Example 19 and using EtOAc-hexane (2:3) as the eluant. Yield 164 mg (95%), mp 196-198° C., off-white crystals. ¹H NMR (CDCl₃, 200 MHz) δ 5.38 (s, 2H, CH₂), 6.66 (dd, J=3.6 and 1.8 Hz, 1H, H-4 in furyl), 7.16-7.18 (m, 1H, Ar), 7.27-7.31 (m, 3H, Ar), 7.78 (m, 1H, H-5 in furyl), 7.86 (br d, J=3.6 Hz, 1H, H-3 in furyl), 8.01 (s, 1H, H-8); HRMS: Found 344.0246, calcd for C₁₆H₁₀Cl₂N₄O 344.0232; Anal. (C₁₆H₁₀Cl₂N₄O) C, H, N.

### EXAMPLE 49

### 2-Chloro-9-(4-fluorophenylmethyl)-6-(2-furyl)-9H-purine

The title compound was produced analogously to that of Example 46 using the compound of Example 20 and using EtOAc-hexane (2:3) as the eluant. Yield 127 mg (77%), mp 152-155° C., pale yellow crystals. ¹H NMR (CDCl₃, 200 MHz) δ 5.38 (s, 2H, CH₂), 6.65 (dd, J=3.6 and 1.8 Hz, 1H, H-4 in furyl), 7.01-7.09 (m, 2H, Ar), 7.24-7.34 (m, 2H, Ar), 7.77 (m, 1H, H-5 in furyl), 7.86 (br d, J=3.6 Hz, 1H, H-3 in furyl), 7.99 (s, 1H, H-8); HRMS: Found 328.0517, calcd for C₁₆H₁₀ClFN₄O 328.0527; Anal. (C₁₆H₁₀ClFN₄O) H, N, C, calcd, 58.46; found 59.18.

### EXAMPLE 50

### 2-Chloro-6-(2-furyl)-9-(4-methyloxyphenylmethyl)-9H-purine

The title compound was produced analogously to that of Example 46 using the compound of Example 21 and using EtOAc-hexane (1:2) as the eluant. Yield 120 mg (74%), mp 171-173° C., colorless crystals. ¹H NMR (CDCl₃, 200 MHz) δ 2.33 (s, 3H, CH₃), 5.35 (s, 2H, CH₂), 6.65 (dd, J=3.6 and 1.8 Hz, 1H, H-4 in furyl), 7.18 (m, 4H, Ar), 7.76 (m, 1H, H-5 in furyl), 7.84 (br d, J=3.6 Hz, 1H, H-3 in furyl), 7.97 (s, 1H, H-8); HRMS: Found 324.0775, calcd for C₁₇H₁₃ClN₄O 324.0778.

### EXAMPLE 51

### 2-Chloro-6-(2-furyl)-9-(4-methoxyphenylmethyl)-9H-purine

The title compound (the preferred embodiment of the invention) was produced analogously to that of Example 46 using the compound of Example 22 and using EtOAc-hexane (1:1) as the eluant. Yield 104 mg (61%), mp 184-186° C., colorless crystals. ¹H NMR (CDCl₃, 200 MHz) δ 3.78 (s, 3H, CH₃), 5.33 (s, 2H, CH₂), 6.65 (dd, J=3.6 and 1.6 Hz, 1H, H-4 in furyl), 6.88 (d, J=8.8 Hz, 2H, Ar), 7.26 (d, J=8.8 Hz, 2H, Ar), 7.76 (m, 1H, H-5 in furyl), 7.84 (dd, J=3.6 and 0.6 Hz, 1H, H-3 in furyl), 7.96 (s, 1H, H-8); ¹³C NMR (CDCl₃, 50 MHz) δ 46.7 (CH₂), 55.2 (CH₃), 112.7 (C-4 in furyl), 114.4 (CH in Ar), 118.6 (C-3 in furyl), 126.4 (C-1 in Ar), 127.1 (C-5), 129.4 (CH in Ar), 144.6 (C-8), 146.5 (C-5 in furyl), 147.1 (C-6), 148.7 (C-2 in furyl), 153.5 (C-4), 154.3 (C-2), 159.7 (C-4 in Ar); MS EI m/z (rel. %): 342/340 (12/38, M⁺), 222 (3), 220 (10), 192 (2), 122 (9), 121 (100), 106 (3), 91 (4), 78 (9), 77 (8). HRMS: Found 340.0735, calcd for C₁₇H₁₃ClN₄O₂ 340.0727; Anal. (C₁₇H₁₃ClN₄O₂) C, H, N.

### EXAMPLE 52

### 2-Chloro-6-(2-furyl)-9-(3-methoxyphenylmethyl)-9H-purine

The title compound was produced analogously to that of Example 46 using the compound of Example 23 and using EtOAc-hexane (2:3) as the eluant. Yield 161 mg (94%), mp 160-161° C., off-white crystals. ¹H NMR (CDCl₃, 200 MHz) δ 3.77 (s, 3H, CH₃), 5.37 (s, 2H, CH₂), 6.65 (dd, J=3.6 and 1.8 Hz, 1H, H-4 in furyl), 6.84-6.88 (m, 3H, Ar), 7.26-7.31 (m, 1H, Ar), 1.77 (m, 1H, H-5 in furyl), 7.85 (br d, J=3.6 Hz, 1H, H-3 in furyl), 8.00 (s, 1H, H-8); ¹²C NMR (CDCl₃, 50 MHz) δ 47.3 (CH₂), 55.3 (CH₃), 112.8 (C-4 in furyl), 113.7 (CH in Ar), 114.0 (CH in Ar), 118.7 (C-3 in furyl), 120.0 (CH in Ar), 127.1 (C-5), 130.3 (CH in Ar), 136.0 (C in Ar), 144.8 (C-8), 146.6 (C-5 in furyl), 147.3 (C-6), 148.7 (C-2 in furyl), 153.6 (C-4), 154.4 (C-2), 160.1 (C-3 in Ar); MS EI m/z (rel. %): 342/340 (33/100, M⁺), 311 (7), 305 (14), 233 (7), 121 (100), 91 (27), 78 (14), 77 (10). HRMS: Found 340.0743, calcd for C₁₇H₁₃ClN₄O₂ 340.0727; Anal. (C₁₇H₁₃ClN₄O₂) C, H, N.

### EXAMPLE 53

### 2-Chloro-6-(2-furyl)-9-(4-methoxy-3-methylphenylmethyl)-9H-purine

The title compound was produced analogously to that of Example 46 using the compound of Example 24 and using EtOAc-hexane (2:3) as the eluant. Yield 117 mg (66%), mp 177-179° C., off-white crystals. ¹H NMR (CDCl₃, 200 MHz) δ 2.17 (s, 3H, CH₃), 3.80 (s, 3H, OCH₃), 5.29 (s, 2H, CH₂), 6.64 (dd, J=3.4 and 1.8 Hz, 1H, H-4 in furyl), 6.78 (d, J=8.0 Hz, 1H, Ar), 7.07-7.14 (m, 2H, Ar), 7.75 (br s, 1H, H-5 in furyl), 7.83 (br d, J=3.4 Hz, 1H, H-3 in furyl), 7.96 (s, 1H, H-8); ¹³C NMR (CDCl₃, 50 MHz) δ 16.2 (CH₃), 47.1 (CH₂), 55.4 (OCH₃), 110.2 (CH in Ar), 112.7 (C-4 in furyl), 118.6 (C-3 in furyl), 125.9 (C in Ar), 126.9 (CH in Ar), 127.2 (C-5), 127.6 (C in Ar), 130.5 (CH in Ar), 144.8 (C-8), 146.5 (C-5 in furyl), 147.2 (C-6), 148.8 (C-2 in furyl), 153.6 (C-4), 154.3 (C-2), 158.0 (C-4 in Ar); MS EI m/z (rel. %): 356/354 (9/28, M⁺), 177 (2), 136 (9), 135 (100), 120 (2), 103 (1), 91 (6). HRMS: Found 354.0872, calcd for C₁₈H₁₅ClN₄O₂ 354.0884; Anal. (C₁₈H₁₅ClN₄O₂) C, H, N.

### EXAMPLE 54

### 2-Chloro-6-(4-methoxyphenyl)-9-(4-methoxyphenylmethyl)-9H-purine

n-Butyllithium (0.69 mL, 1.1 mmol, 1.6 M hexane solution) was added dropwise to a stirred solution of 1-chloro-4-iodobenzene (262 mg, 1.10 mmol) in dry THF (4 ml) under N, at −78° C. After 15 minutes, a 1M solution of anhydrous 4-methoxyphenyl zinc bromide (1.1 mmol) in dry THF (1.2 mL) was added dropwise and the resulting mixture was stirred for 1 hour at −78° C. before the cooling bath was removed and the reaction mixture was allowed to reach ambient temperature. A solution of the compound of Example 22 (309 mg, 1.00 mmol) in dry THF (4 mL) was added followed by a solution of tetrakis(triphenylphosphine)palladium (0) [generated in situ from tris(dibenzylideneacetone)dipalladium chloroform adduct (27 mg, 0.025 mmol) and triphenylphosphine (52 mg, 0.20 mmol)] in dry THF (2 mL). The resulting mixture was heated at 50° C. for 4 hours and cooled to ambient temperature. Saturated aqueous ammonium chloride (10 mL) was added and the aqueous phase extracted with EtOAc (2×25 mL). The combined organic extracts were washed with brine (2×20 mL), dried (MgSO₄), and evaporated. The title product was purified by flash chromatography on silica gel by eluting with EtOAc-hexane (1:3). Yield 231 mg (61%), colorless needles, mp 152-154° C. ¹H NMR (CDCl₃, 200 MHz) δ 3.78 (s, 3H, CH₃), 3.87 (s, 3H, CH₃), 5.32 (s, 2H, C₂), 6.87 (d, J=8.6 Hz, 2H, Ar), 7.02 (d, J=9.0 Hz, 2H, Ar), 7.26 (d, J=8.6 Hz, 2H, Ar), 7.94 (s, 1H, H-8), 8.79 (d, J=9.0 Hz, 2H, Ar), ¹³C NMR (CDCl₃, 50 MHz) δ 46.8 (CH₂), 55.2 (CH₃), 55.3 (CH₃), 113.9 (CH in Ar), 114.3 (CH in Ar), 126.6 (C in Ar), 127.0 (C in Ar), 129.2 (C-5), 129.4 (CH in Ar), 131.7 (CH in Ar), 143.9 (C-8), 153.6 (C-2/C-4/C-6), 153.9 (C-2/C-4/C-6), 155.9 (C-2/C-4/C-6), 159.6 (C in Ar), 162.3 (C in Ar); MS EI m/z (rel. %): 328/380 (12/36, M⁺), 122 (8), 121 (100), 91 (2). HRMS: Found 380.1055. Anal. Calcd for C₂₀H₁₇ClN₄O₂ 380.1045; Found: C, 62.75; H, 4.40; N, 14.54. C₂₀H₁₇ClN₄O₂ requires C, 63.08; H, 4.50; N, 14.71%.

### EXAMPLE 55

### 2-Chloro-9-(methoxyphenylmethyl)-6-(2-thienyl)-9H-purine

A mixture of tris(dibenzylideneacetone)dipalladium chloroform adduct (31 mg, 0.030 mmol) and tri(2-furyl)phosphine (51 mg, 0.22 mmol) in DMF (8 mL) was stirred at ambient temperature under a nitrogen atmosphere for 15 minutes, before the compound of Example 22 (309 mg, 1.00 mmol) and 2-thienyl(tributyl)tin (0.38 mL, 1.20 mmol) were added. The resulting mixture was stirred for 20 hours at 50° C. and evaporated in vacuo. A saturated solution of potassium fluoride in methanol was added to the residue and the mixture was stirred overnight and evaporated in vacuo together with a small amount of silica gel. The residue was added on top of a chromatography column and the product was isolated by flash chromatography on silica gel by eluting with EtOAc-hexane (2:5). Yield 335 mg (94%), colorless needles, mp 183-185° C. ¹H NMR (CDCl₃, 200 MHz) δ 3.78 (s, 3H, CH₃), 5.32 (s, 2H, CH₂), 6.88 (d, J 8.4 Hz, 2H, Ar), 7.20-7.28 (m, 3H, 2H in Ar arid 1H in thienyl), 7.64 (br d, J=5.0 Hz, 1H, thienyl), 7.95 (s, 1H, H-8), 8.62 (br d, J=3.8 Hz, 1H, thienyl); ¹³C NMR (CDCl₃, 50 MHz) δ 47.0 (CH₂), 55.3 (CH₃), 114.4 (CH in Ar), 126.5 (C in Ar), 127.8 (C-5), 128.6 (CH in thienyl), 129.5 (CH in Ar), 131.9 (CH in thienyl), 133.4 (CH in thienyl), 138.5 (C in thienyl), 144.4 (C-8), 151.6 (C-2/C-4/C-6), 153.5 (C-2/C-4/C-6), 154.9 (C-2/C-4/C-6), 159.8 (C in Ar); MS EI m/z (rel. %): 358/356 (9/27, M⁺), 122 (7), 121 (100), 91 (1). HRMS: Found 356.0499. Anal. Calcd for C₁₇H₁₃ClN₄OS; 356.0489. Found: C, 57.55; H, 3.82; N, 15.42. C₁₇H₁₃ClN₄OS requires C, 57.22; H, 3.67; N, 15.70%.

The compounds of the following Examples may be prepared by the following reactions:

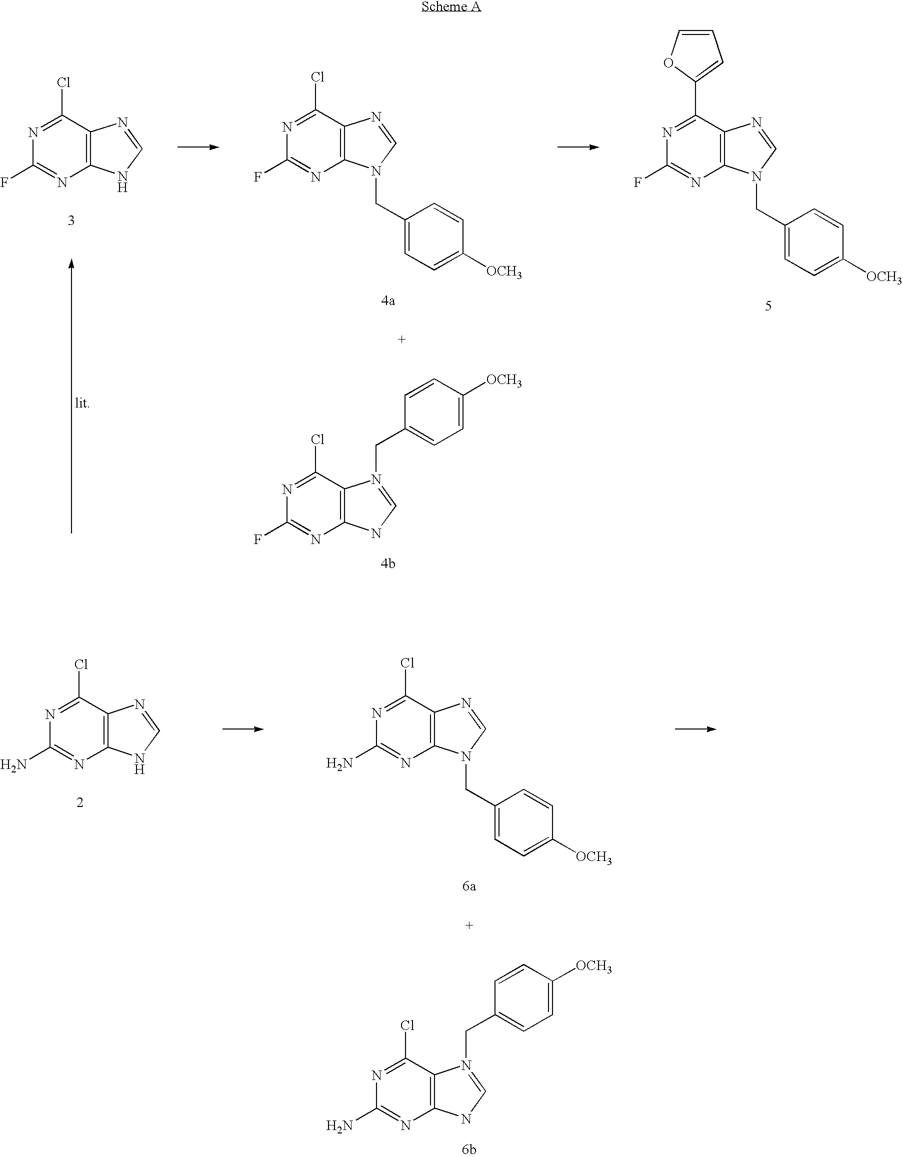

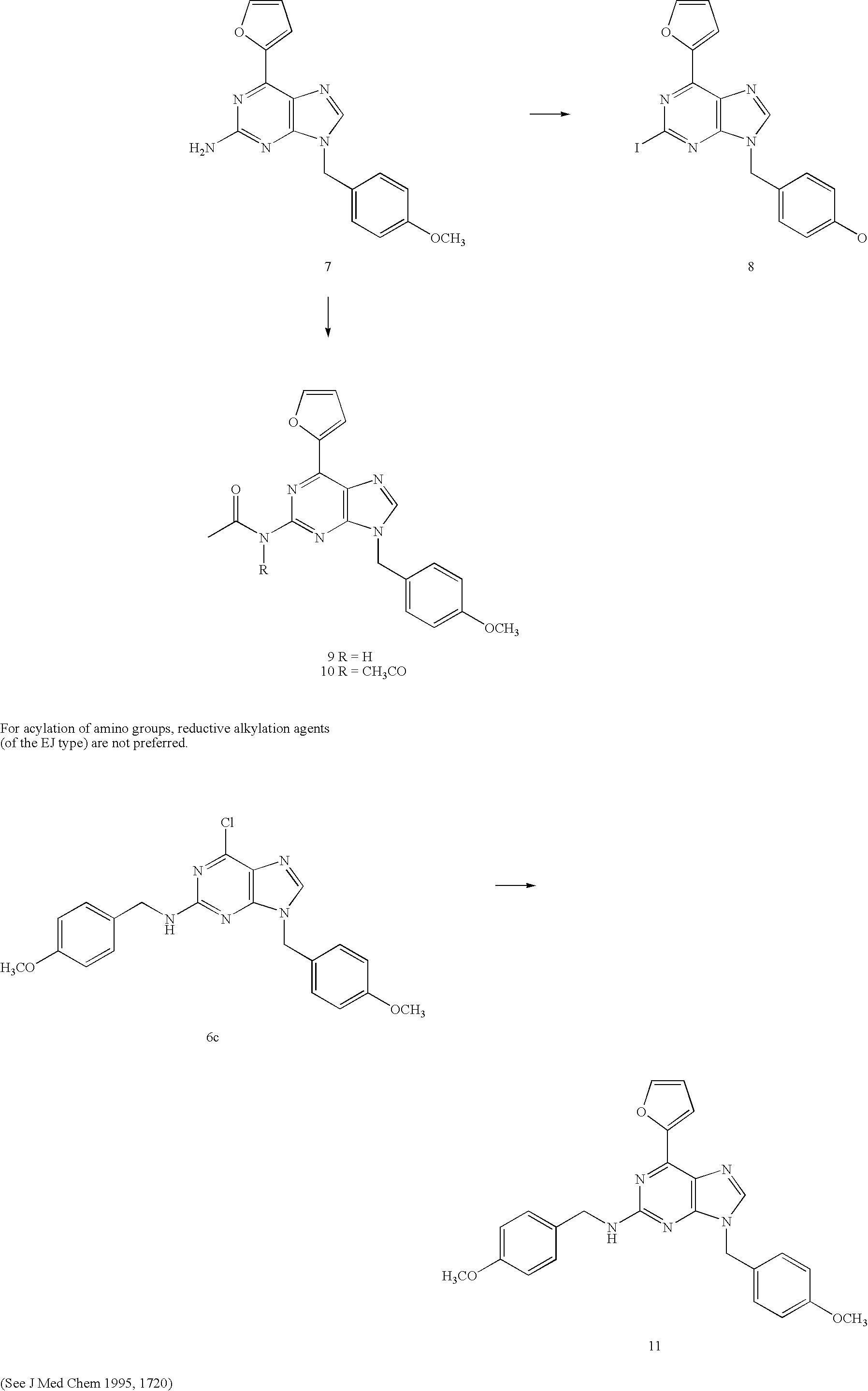

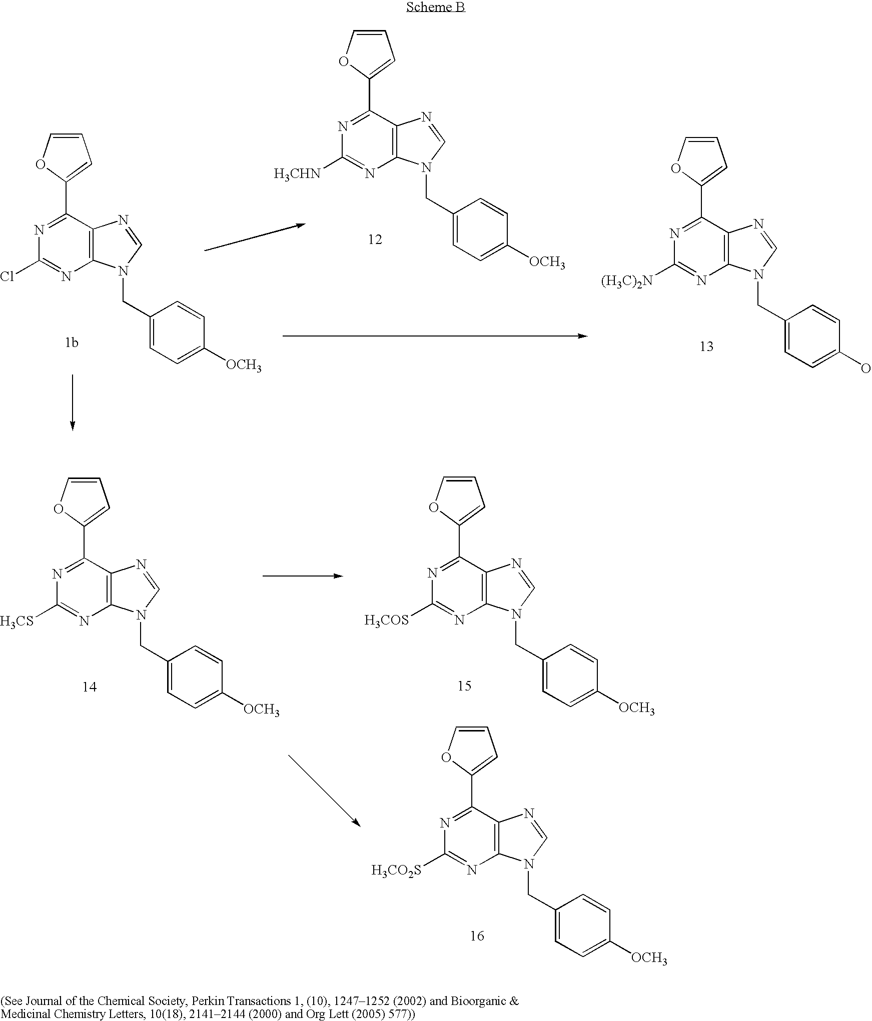

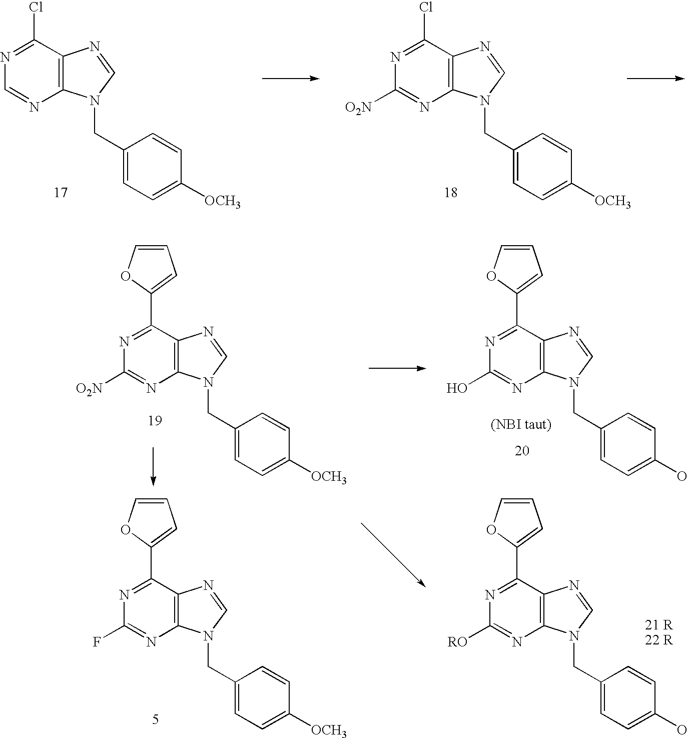

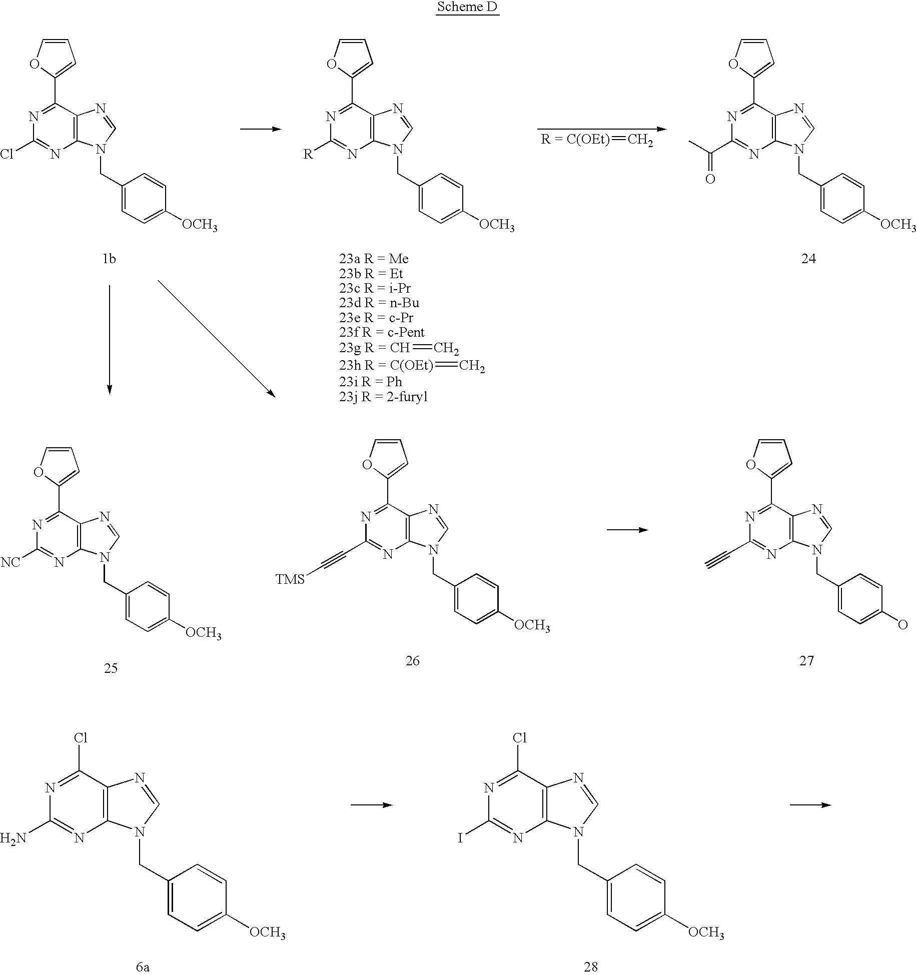

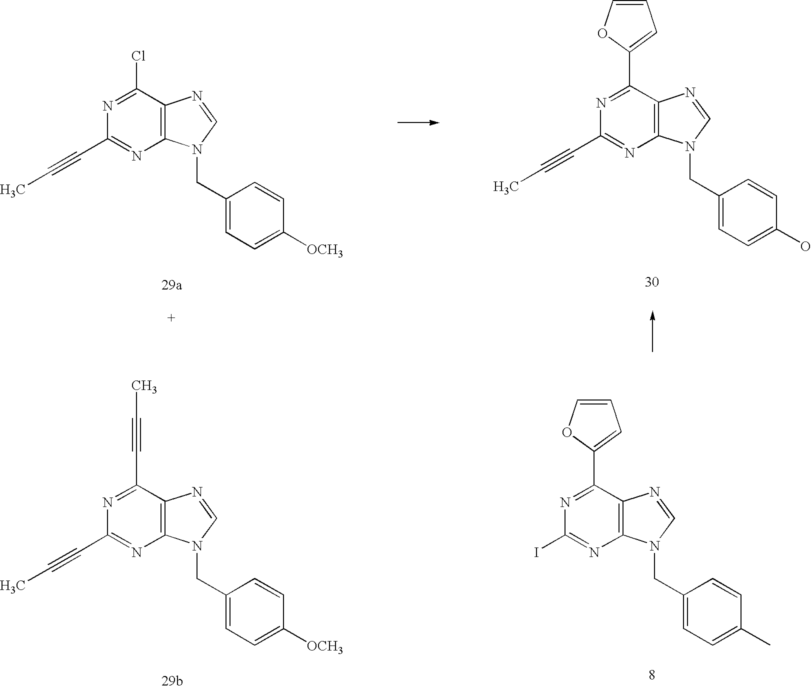

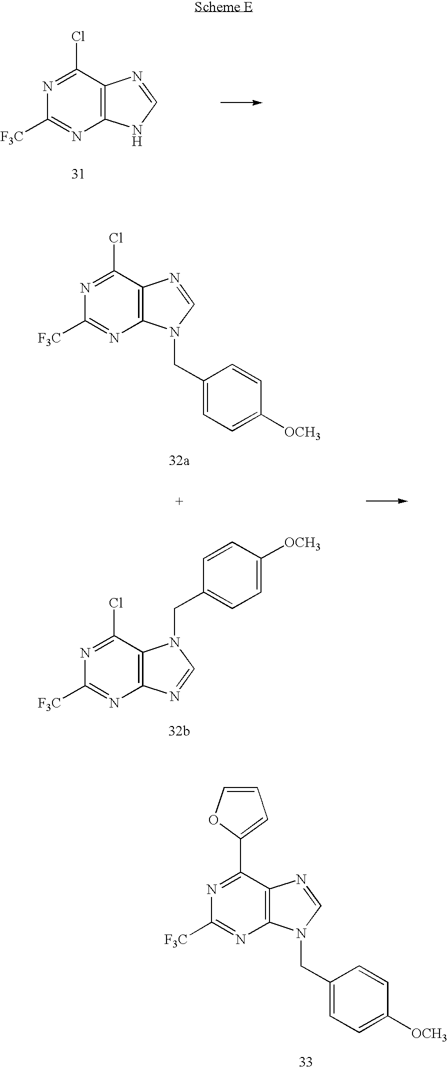

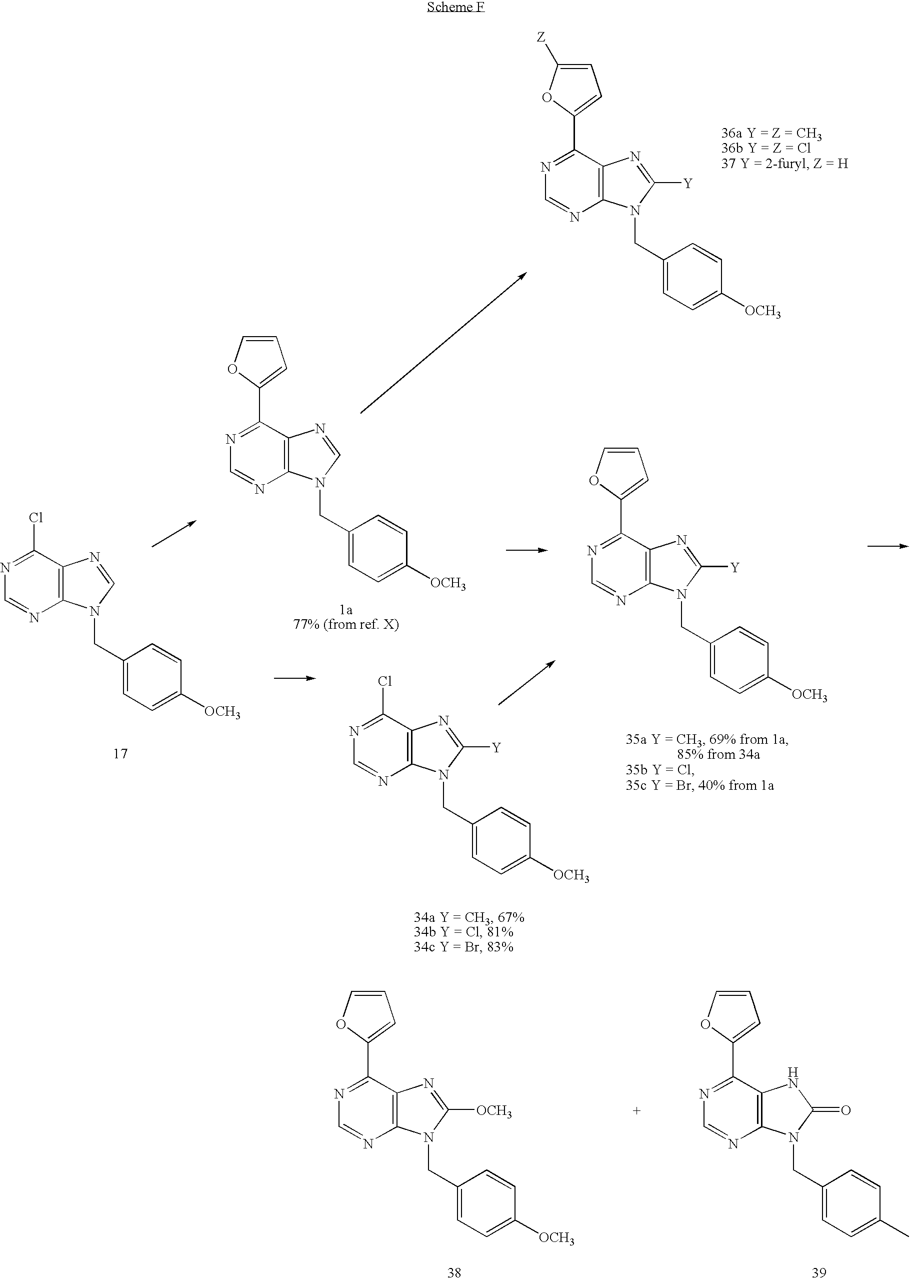

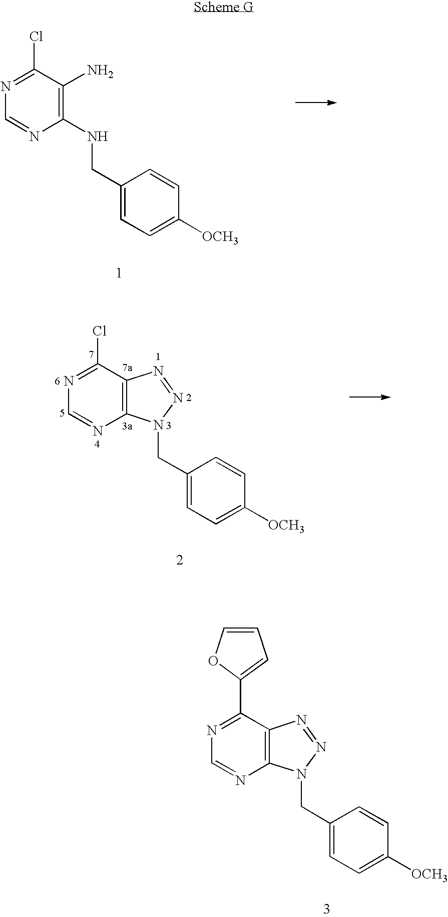

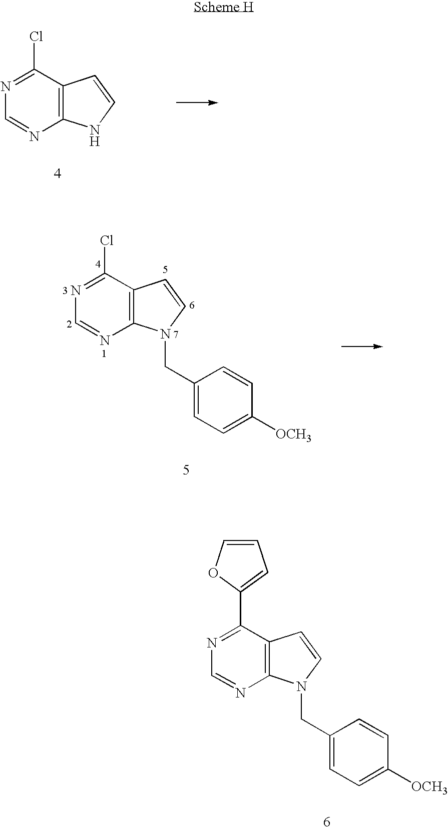

For the following Examples, the ¹H NMR spectra were recorded at 600 MHz with a Bruker AV 600 instrument, at 500 MHz with a Bruker Avance DRX 500 instrument, at 300 MHz with a Bruker Avance DPX 300 instrument or at 200 MHz with a Bruker Avance DPX 200 instrument or a Varian Gemini 200 instrument. The ¹H decoupled ¹³C NMR spectra were recorded at 150, 125, 75 or 50 MHz using instruments mentioned above. ¹⁹F NMR spectra were recorded at 188 MHz with the Avance DPX 200 instrument using CCl₃F in CDCl₃ as referances at 0 ppm. Mass spectra under electron impact conditions (EI) were recorded at 70 eV ionizing voltage with a VG Prospec instrument, and are presented as m/z (% rel. int.). Elemental analyses were performed by Ilse Beetz Mikroanalytisches Laboratorium, Kronach, Germany. Melting points were determined with a C. Reichert melting point apparatus and are uncorrected. DMF was distilled from BaO and stored over 4 Å molsieve. The Grignard reagents used were titrated using salicylaldehyde phenylhydrazone. (See Love, et al. J. Org. Chem. 1999, 64, 3755-3756).

### EXAMPLE 56

### 6-Chloro-2-fluoro-9-(4-methoxyphenylmethyl)-9H-purine

Potassium carbonate (1.38 g, 10.0 mmol) and 6-chloro-2-fluoro-9H-purine (575 mg, 3.33 mmol) was stirred in dry DMF (25 mL) at ambient temperature under N₂ for 20 minutes before 4-methoxyphenylmethyl chloride (904 μL, 6.66 mmol) was added. The resulting mixture was stirred for 16 h, filtered and evaporated in vacuo with a small amount of silica. The isomers were separated by flash chromatography using EtOAc-hexan (1:1) for elution. For the title compound the yield was 327 mg (34%), colorless crystals, mp 90° C. ¹H NMR (CDCl₃, 300 MHz): δ 3.74, (s, 3 H, OCH₃), 5.27 (s, 2H, CH₂), 6.83 (d, J=8.6 Hz, 2H, Ar), 7.23 (d, J=8.6 Hz, 2H, Ar), 8.02 (s, 1H, H-8); ¹³C NMR (CDCl₃ 75 MHz): δ 47.5 (CH₂), 55.2 (OCH₃), 114.5 (CH in Ar), 125.8 (C-1 in Ar), 129.6 (CH in Ar), 130.1 (d, J=5.0 Hz, C-5), 145.5 (d, J=3.2 Hz, C-8), 152.4 (d, J=17.6 Hz, C-6), 153.4 (d, J=17.0 Hz, C-4), 157.2 (d, J=220 Hz, C-2), 159.9 (C-4 in Ar); ¹⁹F NMR (CDCl₃, 188 MHz): −49.78; MS EI m/z (rel. %): 294/292 (7/21, M⁺), 277 (1), 122, (9), 121 (100), 91 (3), 89 (1), 78 (6), 77 (5); HRMS: Found 292.05142, calcd. for C₁₃H₁₀ClFN₄O 292.0527; Anal. (C₁₃H₁₀ClFN₄O) C, H, N.

### EXAMPLE 57

### 2-Fluoro-6-(2-furyl)-9-(4-methoxyphenylmethyl)-9H-purine

Method A. 2-(Tributylstannyl)furan (189 μl, 0.60 mmol) was added to a solution of 6-chloro-2-fluoro-9-(4-methoxyphenylmethyl)-9H-purine (Example 56) (146 mg, 0.50 mmol), and bis(triphenylphosphine)palladium(II) chloride (17.5 mg, 0.025 mmol) in dry DMF (2 mL) and the resulting mixture was heated to 90° C. under N₂ for 16 h, cooled and evaporated in vacuo. A saturated solution of potassium fluoride in methanol (20 mL) was added. The resulting mixture was stirred at ambient temperature for 3 h and evaporated in vacuo together with a small amount of silica. The residue was added at the top of a silica gel column and the product was purified by flash chromatography eluting with EtOAc-hexane (1:1); yield 117 mg (72%), off-white crystals, mp 210-215° C. ¹H NMR (CDCl₃, 200 MHz): δ 3.77 (s, 3H, OCH₃), 5.30 (s, 2H, CH₂), 6.65 (dd, J=3.6 and 1.7 Hz, 1H, H-4 in furyl), 6.87 (d, J=8.7 Hz, 2H, Ar), 7.26 (d, J=8.7 Hz, 2H, Ar), 7.76 (dd, J=1.7 and 0.7 Hz, 1H, H-5 in furyl), 7.83 (dd, J=3.5 and 0.7 Hz, 1H, H-3 in furyl), 7.98 (s, 1H, H-8); ¹³C NMR (CDCl₃, 75 MHz): δ 47.0 (CH₂), 55.3 (OCH₃), 112.8 (C-4 in furyl), 114.5 (CH in Ar), 118.6 (C-3 in furyl), 126.5 (C-1 in Ar), 126.8 (d, J=4.0 Hz, C-5), 129.5 (CH in Ar), 144.8 (d, J=3.0 Hz, C-8), 146.7 (C-5 in furyl), 147.8 (d, J=16.5 Hz, C-6), 149.0 (C-2 in furyl), 154.3 (d, J=17.0 Hz, C-4), 158.9 (d, J=214 Hz, C-2), 159.8 (C-4 in Ar); ¹⁹F NMR (CDCl₃, 188 MHz): −50.50; MS EI m/z (rel. %): 324 (38, M⁺), 162 (2), 122 (9), 121 (100), 91 (3), 78 (5), 77 (5), 65 (1); HRMS: Found 324.1021, calcd. for C₁₇H₁₃FN₄O₂ 324.1023; Anal. (C₁₇H₁₃FN₄O₂) C, H, N.

Method B. TBAF (344 μL, 1.00 M in THF, 0.344 mmol) was added to a solution of 6-(2-furyl)-9-(4-methoxyphenylmethyl)-2-nitro-9H-purine 20 (60 mg, 0.17 mmol) in DMF (0.5 mL) and the mixture was stirred for 1 h at room temperature under N₂ before transfered to a mixture of water (5 mL) and EtOAc (10 ML). The resulting mixture was washed with water (2×10 mL) and brine (2×10 mL), and dried (MgSO₄). The product was purified by flash chromatography on silica gel eluting with EtOAc-hexane (2:1); yield 30 mg (54%).

### EXAMPLE 58

### 2-Amino-6-chloro-9-(4-methoxyphenylmethyl)-9H-purine

Potassium carbonate (2.49 mg, 18.0 mmol) and 2-amino-6-chloro-9H-purine (1.018 g, 6.00 mmol) was stirred in dry DMF (25 mL) at ambient temperature under N₂. After 20 min, 4-methoxyphenylmethyl chloride (1.63 mL, 12.0 mmol) was added. The resulting mixture was stirred for 16 h, filtered and evaporated in vacuo with a small amount of silica. The isomers were separated by flash chromatography eluting with CH₂Cl₂-MeOH (19:1). For the title compound the yield was 917 mg (53%), colorless powdery crystals. ¹H NMR (CDCl₃, 200 MHz); δ 3.78 (s, 3H, CH₃), 5.15 (br s, 4H, CH₂ and NH₂), 6.86 (d, J=8.6 Hz, 2H, Ar), 7.20 (d, J=8.6 Hz, 2H, Ar), 7.68 (s, 1H, H-8); ¹³C NMR (CDCl₃, 50 MHz): δ 46.7 (CH₂), 55.3 (CH₃), 114.4 (CH in Ar), 125.1 (C-5), 126.9 (C-1 in Ar), 129.2 (CH in Ar), 142.0 (C-8), 151.1 (C-4/C-6), 153.7 (C-4/C-6), 159.1 (C-4 in Ar), 159.6 (C-2); MS EI m/z (rel. %); 291/289 (6/18 M⁺), 122 (8), 121 (100), 91 (2), 78 (4), 77 (3); HRMS: Found 289.0730, calcd. for C₁₃H₁₂ClN₅O 289.0732

(See Matsuda et al. Jpn. Kokai Tokkyo Koho (1998), 37 pp. CODEN: JKXXAF JP 10025294 A2 19980127 Heisei).

### EXAMPLE 59

### 6-Chloro-9-(4-methoxyphenylmethyl)-2-(4-methoxyphenylmethylamino)-9H-purine

Colorless needles, mp 161-164° C. ¹H NMR (CDCl₃, 200 MHz): δ 3.815 (s, 3H, CH₃), 3.824 (s, 3H, CH₃), 4.65 (br s, 2H, CH₂NH), 5.18 (s, 2H, CH₂) 5.75 (br s, 1H, NH), 6.84-6.90 (m, 4H, Ar), 7.21-7.34 (m, 4H, Ar), 7.71 (s, 1H, H-8); ¹³C NMR (CDCl₃, 50 MHz) δ 45.4 (CH₂), 46.8 (CH₂), 55.3 (2×CH₃), 113.9 (CH in Ar), 114.3 (CH in Ar), 124.5 (C-5), 127.2 (C in Ar), 128.7 (CH in Ar), 129.4 (CH in Ar), 130.9 (C in Ar), 141.3 (C-8), 151.0 (C-4/C-6), 153.6 (C-4/C-6), 158.69 (C in Ar), 158.74 (C in Ar), 159.6 (C-2); MS EI m/z (rel. %): 411/409 (14/39, M⁺), 290 (7), 288 (22), 136 (2), 122 (9), 121 (100), 91 (3), 78 (6), 77 (5); HRMS: Found 409.1306, calcd. for C₂₁H₂₀ClN₅O₂ 409.1321. Anal. (C₂₁H₂₀ClN₅O₂) C, H, N.

### EXAMPLE 60

### 6-Chloro-9-(4-methoxyphenylmethyl)-2-[di(4-methoxyphenylmethyl)amino]-9H-purine

Colorless powdery crystals, mp 157-159° C. ¹H NMR (CDCl₃, 200 MHz): δ 3.75 (s, 3H, CH₃), 3.78 (s, 6H, 2×CH₃), 4.80 (s, 4H, 2×CH₂), 5.10 (s, 2H, CH₂), 6.74-6.84 (m, 6H, Ar), 7.12-7.21 (m, 6H, Ar), 7.68 (s, 1H, H-8); ¹³C NMR (CDCl₃, 50 MHz): δ 46.8 (CH₂), 49.1 (2×CH), 55.2 (3×CH₃), 113.7 (CH in Ar), 114.2 (CH in Ar), 124.0 (C-5), 129.0 (CH in Ar), 129.4 (CH in Ar), 130.1 (C in Ar), 141.2 (C-8), 150.7 (C-4/C-6), 153.7 (C-4/C-6), 158.7 (3×C in Ar), 159.4 (C-2); MS EI m/z (rel. %): 531/529 (0.5/4, M⁺), 410 (27), 409 (20), 408 (81), 122 (8), 121 (100), 91 (2), 78 (1), 77 (2); HRMS: Found 529.1867, calcd. for C₂₉H₂₈ClN₅O₃ 529.1881. Anal. (C₂₉H₂₈ClN₅O₃) C, H, N.

### EXAMPLE 61

### 2-Amino-6-(2-furyl)-9-(4-methoxyphenylmethyl)-9H-purine

The title compound was prepared by Stille coupling between 2-amino-6-chloro-9-(4-methoxyphenylmethyl)-9H-purine (Example 58) (0.67 mmol) and 2-(tributylstannyl)furan as described for Example 57 (method A). The product was purified by flash chromatography eluting with EtOAc-hexane (1:1) followed by EtOAc-hexane (2:1) and finally pure EtOAc; yield 189 mg (88%), colorless crystals, mp 235-237° C. (dec.). ¹H NMR (CDCl₃, 200 MHz): δ 3.77 (s, 3H, CH₃), 5.10 (br s, 2H, NH₂), 5.19 (s, 2H, CH₂), 6.61 (dd, J=3.6 and 1.6 Hz, 1H, H-4 in furyl), 6.85 (d, J=8.6 Hz, 2H, Ar), 7.22 (d, J=8.6 Hz, 2H, Ar), 7.70 (m, 2H, H1-5 in furyl and H-8), 7.77 (br d, J=3.6 Hz, 1H, H-3 in furyl); ¹³C NMR (DMSO-d₆, 50 MHz): δ 45.1 (CH₂), 55.0 (CH₃), 112.3 (C-4 in furyl), 113.9 (CH in Ar), 116.2 (C-3 in furyl), 121.6 (C-5), 128.6 (CH in Ar), 128.8 (C-4 in Ar), 142.2 (C-8), 145.2 (C-5 in furyl), 145.4 (C-6), 149.3 (C-2 in furyl), 153.6 (C-4), 158.6 (C-2), 160.2 (C in Ar); MS EI m/z (rel. %): 321 (55, M⁺), 122 (8), 121 (100), 91 (3), 78 (5), 77 (5); HRMS: Found 321.1234, calcd. for C₇H₁₅N₅O₂ 321.1226.

### EXAMPLE 62

### 6-(2-Furyl)-2-iodo-9-(4-methoxyphenylmethyl)-9H-purine

A mixture of 2-amino-6-(2-furyl)-9-(4-methoxyphenylmethyl)-9H-purine (Example 61) (80 mg, 0.25 mmol), diiodomethane (2.5 mL) and isoamyl nitrite (670 μL, 5.00 mmol) was heated at 85° C. for 75 min. After cooling, the solution was evaporated in vacuo together with a small amount of silica. The product was purified by flash chromatography on silica gel eluting with EtOAc-hexane (2:1); yield 43 mg (40%), colorless oil. Crystallization from CH₂Cl₂-acetone afforted an analytical sample, colorless crystalline compound, mp 145-148° C. ¹H NMR (CDCl₃, 300 MHz): δ 3.80 (s, 3H, OCH₃), 5.33 (s, 2H, CH₂), 6.65 (dd, J=3.5 and 1.7 Hz, 1H, H-4 in furyl), 6.89 (d, J=8.7 Hz, 2H, Ar), 7.27 (d, J=8.7 Hz, 2H, Ar), 7.77 (d, J=1.7 and 0.7 Hz, 1H, H-5 in furyl), 7.82 (dd, J=3.5 and 0.7 Hz, 1H, H-3 in furyl), 7.91 (s, 1H, H-8); ¹³C NMR (CDCl₃, 75 MHz): δ 46.9 (CH₂), 55.2 (OCH₃), 112.7 (C-4 in furyl), 114.4 (CH in Ar), 118.4 (C-3 in furyl), 119.6 (C-2), 126.5 (C-1 in Ar), 128.0 (C-5), 129.5 (CH in Ar), 144.1 (C-8), 146.5 (C-5 in furyl), 146.6 (C-6), 148.7 (C-2 in furyl), 152.8 (C-4), 159.8 (C-4 in Ar); MS EI m/z (rel. %): 432 (56, M⁺), 305 (2), 184 (2), 122 (8), 121 (100), 106 (2), 91 (3), 78 (8), 77 (7); HRMS: Found 432.0070, calcd. for C₁₇H₁₃IN₄O, 432.0083; Anal. (C₁₇H₁₃IN₄O₂) C, H, N.

### EXAMPLE 63

### N-[6-(2-Furyl)-9-(4-methoxybenzyl)-9H-purin-2-yl]acetamide

A stirred suspension of 2-amino-6-(2-furyl)-9-(4-methoxyphenylmethyl)-9H-purine (Example 61) (40 mg, 0.125 mmol) and trietylamine (174 μg, 1.25 mmol) in toluene (2 mL) was heated at reflux. Acetic anhydride (118 μ1, 1.25 mmol) was added and the reaction mixture was heated at reflux for 22 h, cooled and evaporated in vacuo together with a small amount of silica. The product was purified by flash chromatography on silica gel eluting with CH₂Cl₂-acetone (9:1) followed by CH₂Cl₂-acetone (7:3); yield 47 mg (52%), off-white crystalline compound, mp 167-168° C. ¹H NMR (CDCl₃, 300 MHz): δ 2.55 (s, 3H, CH₃CO), 3.75 (s, 3H, OCH₃), 5.26 (s, 2H, NCH₂), 6.61 (dd, J=3.5 and 1.7 Hz, 1H, H-4 in furyl), 6.85 (d, J=8.7 Hz, 2H, Ar), 7.25 (d, J=8.7 Hz, 2H, Ar), 7.71 (dd, J=1.7 and 0.7 Hz, 1H, H-5 in furyl), 7.75 (dd, J=3.5 and 0.7 Hz, 1H, H-3 in furyl), 7.91 (s, 1H, H-8), 8.27 (br s, 1H, NH); ¹³C NMR (CDCl₃, 75 MHz): δ 25.1 (CH₃CO), 46.9 (NCH₂), 55.3 (OCH₃), 112.6 (C-4 in furyl), 114.4 (CH in Ar), 117.6 (C-3 in furyl), 125.1 (C-5), 127.0 (C-1 in Ar), 129.5 (CH in Ar), 143.7 (C-8), 146.0 (C-5 in furyl), 146.5 (C-6), 149.5 (C-2 in furyl), 152.7 (C-2), 153.0 (C-4), 159.7 (C-4 in Ar), 171.0 (C═O); MS EI m/z (rel. %): 363 (100, M⁺), 362 (9), 348 (6), 322 (4), 321 (21), 320 (3), 122 (9), 121 (99), 91 (3), 78 (2), 77 (3); HRMS: Found 363.1324, calcd. for C₁₉H₁₇N₅O₃ 363.1331; Anal. (C₁₉H₁₇N₅O₃) C, H, N. 2-(N,N-diacetylamino)-6-(2-furyl)-9-(4-methoxyphenylmethyl)-9H-purine (Example 64) was isolated in 39 mg (38%) yield, data see below.

### EXAMPLE 64

### 2-(N,N-diacetylamino)-6-(2-furyl)-9-(4-methoxyphenylmethyl)-9H-purine

A stirred suspension of 2-amino-6-(2-furyl)-9-(4-methoxyphenylmethyl)-9H-purine (Example 61) 160 mg, 0.50 mmol) in toluene (4 mL) was heated at refluxfor 24 h. Acetic anhydride (472 μL, 5.00 mmol) was added. After additional 24 h reflux, the solution was cooled, CH₂Cl₂ and a small amount of silica gel was added and the mixture evaporated in vacuo. The product was isolated by flash chromatography on silica gel eluting with CH₂Cl₂-acetone (9:1); yield 151 mg (74%), colorless crystalline compound, mp 187-189° C. ¹H NMR (CDCl₂, 300 MHz): δ 2.28 (s, 6H, 2×CH₃CO), 3.71 (s, 3H, OCH₃), 5.27 (s, 2H, NCH₂), 6.60 (dd, J=3.5 Hz, J=1.7 Hz, 1H, H-4 in furyl), 6.80 (d, J=8.7 Hz, 2H, Ar), 7.21 (d, J=8.7 Hz, 2H, Ar), 7.70 (dd, J=1.7 and 0.7 Hz, 1H, H-5 in furyl), 7.85 (dd, J=3.5 and 0.7 Hz, 1H, H-3 in furyl), 8.06 (s, 1H, H-8); ¹³C NMR (CDCl₃, 75 MHz): δ 26.3 (2×CH₃CO), 47.0 (NCH₂), 55.1 (OCH₃), 112.7 (C-4 in furyl), 114.3 (CH in Ar), 118.7 (C-3 in furyl), 126.5 (C-1 in Ar), 127.4 (C-5), 129.4 (CH in Ar), 145.6 (C-8), 146.3 (C-5 in furyl), 147.2 (C-6), 148.7 (C-2 in furyl), 153.2 (C-2/C-4), 153.4 (C-4/C-2), 159.7 (C-4 in Ar), 172.1 (2×C═O); MS EI m/z (rel. %); 405 (34, M⁺), 364 (16), 363 (70), 362 (6), 349 (17), 348 (80), 321 (12), 122 (9), 121 (100), 91 (3), 78 (4), 77 (4); HRMS: Found 405.1432, calcd. for C₂₁H₁₉N₉O₄ 405.1437; element

### EXAMPLE 65

### 6-(2-Furyl)-2-(4-methoxyphenylmethylamino)-9-(4-methoxyphenylmethyl)-9H-purine

The title compound was prepared by Stille coupling on 6-chloro-9-(4-methoxyphenylmethyl)-2-(4-methoxyphenylmethylamino)-9H-purine (Example 59) (0.70 mmol) as described for Example 57 above. EtOAc-hexane (1:1) followed by EtOAc-hexane (2:1) were used for flash chromatography; yield 262 mg (85%), colorless crystals, mp 153-154° C. ¹H NMR (CDCl₃, 200 MHz): δ 3.75 (5, 3H, CH₂), 3.76 (s, 3H, CH₃), 4.63 (br d, J=5.8 Hz, 2H, CH₂NH), 5.16 (s, 2H, CH₂) 6.09 (br s, 1H, NH), 6.59 (dd, J=3.4 and 1.6 Hz, 1H, H-4 in furyl), 6.78-6.84 (m, 4H, Ar), 7.16-7.31 (m, 4H, Ar), 7.70 (m, 1H, H-5 in furyl), 7.71 (s, 1H, H-8), 7.79 (br d, J=3.4 Hz, 1H, H-3 in furyl); ¹³C NMR (CDCl₃, 50 MHz): δ 45.3 (CH₂), 46.4 (CH₂), 55.2 (2×CH₃), 112.5 (C-4 in furyl), 113.8 (CH in Ar), 114.2 (CH in Ar), 117.7 (C-3 in furyl), 122.1 (C-5), 127.5 (C-1 in Ar), 128.7 (CH in Ar), 129.4 (CH in Ar), 131.2 (C-1 in Ar), 141.9 (C-8), 145.8 (C-6), 145.9 (C-5 in furyl), 148.6 (C-2 in furyl), 154.3 (C-4), 158.1 (C-2/C-4 in Ar), 158.6 (C-2/C-4 in Ar), 159.4 (C-2/C-4 in Ar); MS EI m/z (rel. %): 441 (100, M⁺), 426 (4), 321 (14), 320 (67), 185 (4), 136 (6), 121 (64), 91 (1), 78 (2), 77 (2); HRMS: Found 441.1780, calcd. for C₂₅H₂₃N₅O₃ 441.1801. Anal: Found: 68.02; H, 5.28; N, 15.09. C₁₇H₁₅N₅O₂ requires C, 68.04; H, 5.25; N, 15.86%.

### EXAMPLE 66

### 6-(2-Furyl)-9-(4-methoxyphenylmethyl)-2-(N-methylamino)-9H-purine

A solution of 2-chloro-6-(2-furyl)-9-(4-methoxyphenylmethyl)-9H-purine (Example 50) (112 mg, 0.33 mmol) in THF (3 mL) and 40% methylamine in water (3 mL) was stirred under N₂ in a sealed tube at 85° C. for 15 h. After cooling the solution was evaporated in vacuo together with a small amount of silica. The product was purified by flash chromatography on silica gel eluting with CH₂Cl₂-acetone (19:1) followed by CH₂Cl₂-acetone (9:1); yield 95 mg (86%), off-white crystalline compound, mp 144-145° C. ¹H NMR (CDCl₃, 300 MHz): δ 3.06 (d, 3H, NCH₃), 3.76 (s, 3H, OCH₄), 5.20 (s, 2H, NCH₂), 5.32 (br s, 1H, NH), 6.58 (dd, J=3.5 and 1.8 Hz, 1H, H-4 in furyl), 6.84 (d, J=8.7 Hz, 2H, Ar), 7.25 (d, J=8.7 Hz, 2H, Ar), 7.67 (br s, H-5 in furyl and H-8), 7.73 (dd, J=3.5 and 0.7 Hz, 1H, H-3 in furyl); ¹³C NMR (CDCl₃, 75 MHz): δ 28.8 (NCH₃), 46.2 (NCH₂), 55.3 (OCH₃), 112.2 (C-4 in furyl), 114.2 (CH in Ar), 116.4 (C-3 in furyl), 122.4 (C-5), 127.9 (C-1 in Ar), 129.4 (CH in Ar), 140.9 (C-8), 145.1 (C-5 in furyl), 146.5 (C-6), 150.0 (C-2 in furyl), 154.1 (C-4), 159.5 (C-4 in Ar), 160.2 (C-2); MS EI m/z (rel. %): 335 (100, M⁺), 334 (3), 320 (1), 214 (3), 122 (8), 121 (81), 106 (1), 91 (2), 78 (4), 77 (4); HRMS: Found 335.1378, calcd. for C₁₈H₁₇N₅O₂ 335.1382; Anal. (C₁₉H₁₇N₅O₂) C, H, N.

### EXAMPLE 67

### 2-(N,N-Dimethylamino)-6-(2-furyl)-9-(4-methoxyphenylmethyl)-9H-purine

Triethylamine (1.05 mL, 7.50 mmol) was added to a mixture of glycine methyl ester hydrochloride (4.71 mg, 3.75 mmol) and 2-chloro-6-(2-furyl)-9-(4-methoxyphenylmethyl)-9H-purine (Example 50) (112 mg, 0.33 mmol) in DMF (3 mL) and the reaction mixture was heated in a sealed tube under nitrogen at 85° C. for 15 h. After cooling the precipitate was filtered off and the solution evaporated in vacuo. The product was dissolved in CH₂Cl₂ (15 mL), the precipitate was filtered off and the solution was evaporated in vacuo together with a small amount of silica. The product was purified by flash chromatography on silica gel eluting with CH₂Cl₂-acetone (19:1); yield 95 mg (82%), colorless crystalline compound, mp 163-164° C. ¹H NMR (CDCl₃, 300 MHz): δ 3.27 [s, 6H, N(CH₂)₂], 3.75 (s, 3H, OCH₃), 5.18 (s, 2H, NCH₂), 6.57 (dd, J=3.4 and 1.7 Hz, 1H, H-4 in furyl), 6.83 (d, J=8.7 Hz, 2H, Ar), 7.25 (d, J=8.7 Hz, 2H, Ar), 7.62 (br d, J=3.4 Hz, H-3 in furyl), 7.67 (s, 1H, H-8), 7.68-7.72 (br s, 1H, H-5 in furyl); ¹³C NMR (CDCl₃, 75 MHz): δ 37.4 [N(CH₃)₂], 46.1 (NCH₂), 55.2 (OCH₃), 111.9 (C-4 in furyl), 114.2 (CH in Ar), 115.2 (C-3 in furyl), 121.3 (C-5), 128.1 (C-1 in Ar), 129.4 (CH in Ar), 140.9 (C-8), 145.1 (C-5 in furyl), 146.1 (C-6), 151.2 (C-2 in furyl), 154.3 (C-4), 159.4 (C-4 in Ar), 159.7 (C-2) ; MS EI m/z (rel. %): 349 (100, M⁺), 348 (3), 334 (7), 321 (3), 320 (14), 306 (2), 305 (2), 228 (4), 122 (4), 121 (52), 91 (2); HRMS: Found 349.1530, calcd. for C₁₉H₁₉N₅O₂ 349.1539; Anal. (C₁₉H₁₉N₅O₂) C, H, N.

### EXAMPLE 68

### 6-(2-Furyl)-9-(4-methoxyphenylmethyl)-2-methylthio-9H-purine

A solution of 2-chloro-6-(2-furyl)-9-(4-methoxyphenylmethyl)-9H-purine (Example 50) (341 mg, 1.00 mmol) and sodium methythiolate (1.05 g, 15.0 mmol) in dry DMF (8 mL) were heated at 90° C. under N₂ for 1.5 h and cooled. The reaction mixture was diluted with water (5 mL) and EtOAc (10 mL), extracted with EtOAc (4×25 mL), washed with brine (2×50 mL), and dried (MgSO₄). The product was purified by flash chromatography on silica gel eluting with EtOAc-hexane (1:1) followed by EtOAc-hexane (1:1); yield 255 mg (72%), pale yellow crystals, mp 147-148° C. ¹H NMR (CDCl₃, 300 MHz): δ 2.62 (s, 3H, SCH₃), 3.71 (s, 3H, OCH₃), 5.24 (s, 2H, CH₂), 6.57 (dd, J=3.5 and 1.8 Hz, 1H, H-4 in furyl), 6.80 (d, J=8.7 Hz, 2H, Ar), 7.21 (d, J=8.7 Hz, 2H, Ar), 7.69-7.70 (m, 1H, H-5 in furyl), 7.71 (dd, J=3.5 and 0.7 Hz, 1H, H-3 in furyl), 7.84 (s, 1H, H-8); ¹³C NMR (CDCl₃, 75 MHz): δ 14.6 (SCH₃), 46.5 (CH₂), 55.1 (OCH₃), 112.3 (C-4 in furyl), 114.2 (CH in Ar), 117.0 (C-3 in furyl), 125.4 (C-5), 127.1 (C-1 in Ar), 129.4 (CH in Ar), 142.9 (C-8), 145.6 (C-6), 145.8 (C-5 in furyl), 149.7 (C-2 in furyl), 152.8 (C-4), 159.5 (C-4 in Ar), 165.8 (C-2); MS EI m/z (rel. %): 352 (56, M⁺), 351 (4), 231 (8), 122 (9), 121 (100), 106 (1), 91 (3), 78 (4), 77 (5); HRMS: Found 352.0983, calcd. for C₁₈H₁₆N₄O₂S 352.0994; Anal. (C17H15N5O2) C, H, N.

### EXAMPLE 69

### 6-(2-Furyl)-9-(4-methoxyphenylmethyl)-2-methylsulfinyl-9H-purine

A solution of 6-(2-furyl)-9-(4-methoxyphenylmethyl)-2-methylthio-9H-purine (Example 68) (88 mg, 0.25 mmol) in CH₂Cl₂ (2 mL) was cooled to −10° C., before a cold solution of m-CPBA (21.6 mg, 0.125 mmol, 0.5 equiv.) in CH₂Cl₂ (1 mL) was added drop wise. The mixture was stirred for 30 minutes. Additional m-CPBA (21.6 mg, 0.125 mmol, 0.5 equiv.) in CH₂Cl₂ (1 mL) was added and the mixture stirred for 30 minutes. The solution was washed with 5% aq. Na₂S₂O₄ (10 mL), sat. aq. NAHCO₃ (10 mL) and brine (10 mL). The dried (MgSO₄) solution was evaporated together with a small amount of silica and added at the top of a silica gel column. The product was purified by flash chromatography eluting with EOAc-hexane (4:1) followed by CH₂Cl₂-MeOH (19:1); yield 64 mg (70%), pale yellow oil. ¹H NMR (CDCl₃, 300 MHz); δ 3.01 (s, 3H, SOCH₃), 3.77 (s, 3H, OCH₃), 5.45 (s, 2H, CH₂), 6.65 (dd, J=3.5 and 1.7 Hz, 1H, H-4 in furyl), 6.87 (d, J=8.7 Hz, 2H, Ar), 7.29 (d, J=8.7 Hz, 2H, Ar), 7.77 (dd, J=1.7 and 0.6 Hz, 1H, H-5 in furyl), 7.86 (dd, J=3.5 and 0.6 Hz, 1H, H-3 in furyl), 8.08 (s, 1H, H-8); ¹³C NMR (CDCl₃, 75 MHz): δ 40.7 (SOCH₃), 47.2 (CH₂), 55.3 (OCH₃), 112.8 (C-4 in furyl), 114.5 (CH in Ar), 118.7 (C-3 in furyl), 126.4 (C-1 in Ar), 128.3 (C-5), 129.8 (CH in Ar), 145.6 (C-8), 146.2 (C-6), 146.7 (C-5 in furyl), 149.1 (C-2 in furyl), 153.0 (C-4), 159.9 (C-4 in Ar), 166.8 (C-2); MS EI m/z (rel. %): 368 (6, M⁺), 367 (3), 352 (9), 322 (3), 305 (4), 249 (13), 122 (9), 121 (100), 91 (4), 78 (5), 77 (6); HRMS: Found 368.0946, calcd. for C₁₈H₁₆N₄O₃S 368.0943; Anal: Found: C, 57.10; H, 4.55; N, 15.05. C₁₈H₁₆N₄O₃S requires C, 58.68; H, 4.38; N, 15.21%.

### EXAMPLE 70

### 6-(2-Furyl)-9-(4-methoxyphenylmethyl)-2-methylsulfonyl-9H-purine

A solution of 6-(2-furyl)-9-(4-methoxyphenylmethyl)-2-methylthio-9H-purine (Example 68) (70 mg, 0.20 mmol) in CH₂Cl₂ (3 mL) was cooled to 0° C. before a cold solution of m-CPBA (77.7 mg, 0.45 mmol, 2.25 equivs.) in CH₃Cl₂ (3 mL) was added drop wise. The mixture was stirred at ambient temperature for 21 h before cold 10% aq. NA₂S₂O₄ (6 mL) was added. After 15 min, the mixture was dilluted with CH₂Cl₂ (10 mL), the phases were separated and the organic layer was washed with sat. aq. NaHCO₃ (2×10 mL) and brine (20 mL). The dried (MgSO₄) solution was evaporated together with a small amount of silica and added at the top of a silica gel column. The product was purified by flash chromatography eluting with EtOAc-hexane (4:1); yield 50 mg (65%), colorless crystalline compound, mp 184-185° C. ¹H NMR (CDCl₃, 300 MHz): δ 3.45 (s, 3H, SO₂CH₃), 3.76 (s, 3H, OCH₃), 5.41 (s, 2H, CH₂), 6.65 (dd, J=3.5 and 1.7 Hz, 1H, H-4 in furyl), 6.85 (d, J=8.7 Hz, 2H, Ar), 7.27 (d, J=8.7 Hz, 2H, Ar), 7.77-7.79 (m, H-5 in furyl), 7.84 (br d, J=3.5, 1H, H-3 in furyl), 8.17 (s, 1H, H-8); ¹³C NMR (CDCl₃, 75 MHz): δ 39.7 (SO₂CH₃), 47.5 (CH₂), 55.3 (OCH₃), 112.9 (C-4 in furyl), 114.6 (CH in Ar), 119.1 (C-3 in furyl), 126.1 (C-1 in Ar), 129.2 (C-5), 129.8 (CH in Ar), 146.2 (C-6), 146.8 (C-8), 147.2 (C-5 in furyl), 149.0 (C-2 in furyl), 152.2 (C-4), 159.5 (C-2), 159.9 (C-4 in Ar); MS EI m/z (rel. %): 384 (45, M⁺), 383 (7), 369 (3), 305 (2), 122 (9), 121 (100), 91 (2), 78 (2), 77 (3); HRMS: Found 384.0893, calcd. for C₁₈H₁₆N₄O₄S 384.0892; Anal. (C₁₈H₁₆N₄O₄S) C, H, N.

### EXAMPLE 71

### 6-Chloro-9-(4-methoxyphenylmethyl)-2-nitro-9H-purine

A nitrating mixture was prepared by adding cold 2,2,2-trifluoroacetic anhydride (890 μL, 6.40 mmol) over 2 minutes to a solution of tetrabutylammonium nitrate (1.95 g, 6.40 mmol) in dry CH₂Cl₂ (15 mL) at 0° C. After 15 minutes the solution was added to 6-chloro-9-(4-methoxyphenylmethyl)-9H-purine (Example 7) (1.10 g, 4.00 mmol) in dry CH₂Cl₂ (15 mL) at 0° C. After 3 h the reaction mixture was poured into cold aqueous NAHCO₃ (100 mL) and Et₂O (100 mL). The aqueous phase was extracted with Et₂O (2×100 ml), washed with water (2×100 mL) and brine (100 mL), and dried (MgSO₄). The mixture was evaporated in vacuo and recrystallized from CH₂Cl₂-hexane; yield 735 mg (57%) [purity>95%], which was used without further purifications. A second recrystallization afforted analytically pure sample; yellow crystalline compound, mp 181-182° C. ¹H NMR (CDCl₁, 300 MHz): δ 3.78 (s, 3H, OCH₁), 5.45 (s, 2H, CH₂), 6.89 (d, J=8.7 Hz, 2H, Ar), 7.31 (d, J=8.7 Hz, 2H, Ar), 8.28 (s, 1H, H-8): ¹³C NMR (CDCl₃, 75 MHz): δ 48.4 (CH₂), 55.4 (OCH₃), 114.8 (CH in Ar), 125.1 (C-1 in Ar), 130.0 (CH in Ar), 134.3 (C-5), 148.6 (C-8), 151.9 (C-4), 152.2 (C-6/C-2), 153.4 (C-2/C-6), 160.3 (C-4 in Ar); MS EI m/z (rel. %): 321/319 (8/26, M⁺), 304 (3), 303, (1), 302 (11), 273 (2), 272 (6), 237 (2), 136 (1), 122 (9), 121 (100), 91 (3), 89 (1), 78 (6), 77 (5); HRMS: Found 319.0467 calcd. for C₁₃H₁₀ClN_{b}O₃ 319.0472; Anal. (C₁₃H₁₀ClN₅O₂) C, H, N.

### EXAMPLE 72

### 6-(2-Furyl)-9-(4-methoxyphenylmethyl)-2-nitro-9H-purine

The title compound was prepared by Stille coupling between 6-chloro-9-(4-methoxyphenylmethyl)-2-nitro-9H-purine (Example 71) (335 mg, 1.05 mmol) and 2-(tributylstannyl)furan (397 μL, 1.26 mmol) as described for compound (Example 57) (method A). The product was purified by flash chromatography eluting with EtOAc-hexane (1:2) followed by CH₂Cl₂-acetone (19:1); yield 257 mg (70%), yellow crystalline compound, mp 182-183° C. ¹H NMR (CDCl₃, 300 MHz): δ 3.77 (s, 3H, OCH₃), 5.43 (s, 2H, CH₂), 6.67 (dd, J=3.6 and 1.7 Hz, 1H, H-4 in furyl), 6.87 (d, J=8.7 Hz, 2H, Ar), 7.30 (d, J=8.7 Hz, 2H, Ar), 7.80 (dd, J=1.7 and 0.7 Hz, 1H, H-5 in furyl), 7.94 (dd, J=3.6 and 0.7 Hz 1H, H-3 in furyl), 8.22 (s, 1H, H-8); ¹³C NMR (CDCl₃, 125 MHz): δ; 47.7 (CH₂), 55.3 (OCH₃), 113.2 (C-4 in furyl), 114.7 (CH in Ar), 120.1 (C-3 in furyl), 125.9 (C-1 in Ar), 129.9 (CH in Ar), 130.2 (C-5), 146.6 (C-6), 147.5 (C-5 in furyl), 147.6 (C-8), 148.4 (C-2 in furyl), 152.5 (C-4), 155.3 (C-2), 160.0 (C-4 in Ar); MS EI m/z (rel. %): 351 (25, M⁻), 334 (1), 322 (2), 321 (6), 305 (1), 122 (9), 121 (100), 91 (3), 78 (3), 77 (4); HRMS: Found 351.0956, calcd. for C₁₇H₁₃N₅O₄ 351.0968; Anal. (C₁₇H₁₃N₅O₄) C, H, N.

### EXAMPLE 73

### 6-(2-Furyl)-2-hydroxy-9-(4-methoxyphenylmethyl)-9H-purine

A mixture of 6-(2-furyl)-9-(4-methoxyphenylmethyl)-2-nitro-9H-purine (Example 72) (176 mg, 0.50 mmol) in THF (6 mL) was heated at 50° C. until the purine dissolved, and cooled to ambient temperature. Tetrabutylammonium hydroxide (670 μL, 1.0 mmol, 40% in water, 1.5 M) was added drop wise over 1 min. The resulting mixture was stirred at ambient temperature for 48 h. Sat. aq. NH₄Cl (20 mL) was added and the mixture extracted with EtOAc (2×20 mL) and CH₂Cl₂ (2×20 mL). The combined organic extracts were washed with water (20 mL) and brine (20 mL). The water phase was extracted with CH₂Cl₂ (2×20 mL) The combined organic extracts were dried (MgSO₄) and evaporated in vacuoe. The product was purified by flash chromatography on silica gel eluting with CH₂Cl₂-MeOH (19:1) followed by CH₂Cl₂-MeOH (9:1); yield 109 mg (68%), pale yellow crystalline compound. ¹H NMR (DMSO-d₆, 300 MHz) δ 3.72 (s, 3H, OCH₄), 5.24 (s, 2H, NCH₂), 6.80 (dd, J=3.5 and 1.7 Hz, 1H, H-4 in furyl), 6.90 (d, J=8.7 Hz, 2H, Ar), 7.29 (d, J=8.7 Hz, 2H, Ar), 7.79 (dd, J=3.5 and 0.7 Hz, H-3 in furyl), 7.90 (dd, J=1.7 and 0.7 Hz, H-5 in furyl), 8.37 (s, 1H, H-8), 11.8 (br s, 1H, NH/OH); ¹³C NMR (CDCl₃, 75 MHz) δ 46.1 (NCH₂), 55.2 (OCH₃), 113.4 (C-4 in furyl), 114.4 (C-3 in Ar), 118.8 (C-5), 120.1 (C-3 in furyl), 127.0 (C-1 in Ar), 129.5 (C-2 in Ar), 137.4 (C-6/C-2), 143.7 (C-2 in furyl), 145.8 (C-8), 147.8 (C-5 in furyl), 158.2 (C-2/C-6), 159.5 (C-4), 159.7 (C-4 in Ar); MS EI m/z (rel. %) 322 (39, M⁻), 321 (1), 122 (7), 121 (100), 91 (3), 78 (6), 77 (6); HRMS Found 322.1069 calcd. for C₁₇H₁₄N₄O₃ 322.1066.

### EXAMPLE 74

### 6-(2-Furyl)-2-methoxy-9-(4-methoxyphenylmethyl)-9H-purine

Method A. A mixture of 2-chloro-6-(2-furyl)-9-(4-methoxyphenylmethyl)-9H-purine (Example 50) (299 mg, 0.88 mmol) in a 0.7 M solution of sodium methoxide in methanol (15 mL) was stirred at ref lux under N₂-atm. for 4 h, cooled and poured into a beaker containing cold sat. aq. NH₄Cl. Most of the methanol was removed by evaporation in vacuo and the aqueous mixture was extracted with EtOAc (5×20 mL). The combined organic layers were dried (MgSO₄) and evaporated in vacuo. The product was purified by flash chromatography on silica gel eluting with EtOAc-hexane (2:1); yield 223 mg (75%), colorless crystals, mp 167-169° C. ¹H NMR (CDCl₃, 200 MHz): δ 3.74 (s, 3H, CH₃), 4.08 (s, 3H, CH₃), 5.25 (s, 2H, CH₂), 6.60 (m, 1H, H-4 in furyl), 6.83 (d, J=8.6 Hz, 2H, Ar), 7.24 (d, J=8.6 Hz, 2H, Ar), 7.71 (br s, 1H, furyl), 7.72 (s, 1H, furyl/H-8), 7.84 (s, 1H, furyl/H-8); ¹³C NMR (CDCl₃, 50 MHz): δ 46.6 (CH₂), 55.1 (CH₃), 55.3 (CH₃), 112.3 (C-4 in furyl), 114.3 (CH in Ar), 116.7 (C-3 in furyl), 124.6 (C-5), 127.2 (C-1 in Ar), 129.3 (CH in Ar), 143.0 (C-8), 145.9 (C-5 in furyl), 147.0 (C-2 in furyl/C-6), 150.2 (C-2 in furyl/C-6), 154.2 (C-4), 159.6 (C-4 in Ar), 162.0 (C-2); MS EI m/z (rel. %): 336 (56, M⁺), 321 (2), 122 (8), 121 (100), 91 (2), 78 (4), 77 (4); HRMS: Found 336.1237, calcd. for C₁₈H₁₆N₄O₃ 336.1222. Anal. (C₁₈H₁₆N₄O₃): C, H, N.

### EXAMPLE 75

### 2-Ethoxy-6-(2-furyl)-9-(4-methoxyphenylmethyl)-9H-purine

A mixture of 6-(2-furyl)-9-(4-methoxyphenylmethyl)-2-nitro-9H-purine (Example 72) (88 mg, 0.25 mmol) and KF (781 mg, 13.9 mmol) in ethanol (10 mL) was heated to 50° C. for 24 h and at 70° C. for additional 24 h. The solution was cooled to ambient temperature and evaporated in vacuo and the product was purified by flash chromatography on silica gel eluting with CH₂Cl₂-acetone (19:1); yield 72 mg (82%), colorless crystalline compound, mp 137-138° C. ¹H NMR (CDCl₃, 200 MHz) δ 1.47 (t, J=7.1 Hz, 3H, CH₃), 3.77 (s, 3H, OCH₃), 4.53 (q, J=7.1 Hz, 2H, OCH₂), 5.27 (s, 2H, NCH₂), 6.61 (dd, J=3.3 and 1.9 Hz, 1H, H-4 in furyl), 6.85 (d, J=8.7 Hz, 2H, Ar), 7.25 (d, J=8.7 Hz, 2H, Ar), 7.72-7.73 (m, 2H, H-3 and H-5 in furyl), 7.85 (s, 1H, H-8) ; ¹³C NMR (CDCl₃, 75 MHz) δ 14.5 (CH₃), 46.5 (NCH₂), 55.2 (OCH₃), 63.6 (OCH₂), 112.3 (C-4 in furyl), 114.3 (CH in Ar), 116.6 (C-3 in furyl), 124.5 (C-5), 127.3 (C-1 in Ar), 129.4 (CH in Ar), 142.9 (C-8), 145.9 (C-5 in furyl), 147.1 (C-6), 150.2 (C-2 in furyl), 154.3 (C-4), 159.6 (C-4 in Ar), 161.7 (C-2) ; MS EI m/z (rel. %) 350 (41, M⁺), 349 (3), 336 (3), 335 (12), 322 (3), 321 (3), 122 (9), 121 (100); HRMS Found 350.1371, calcd. for C₁₉H₁₈N₄O₃ 350.1379.

### EXAMPLE 76

### 6-(2-Furyl)-9-(4-methoxyphenylmethyl)-2-methyl-9H-purine

Zinc bromide (1.41 mL, 1.40 mmol, 0.995 M in THF) was added dropwise to a stirred solution of methyllithium (1.0 mL, 1.4 mmol, 1.4 M in diethyl ether) at −78° C. under N₂. After stirring for 1 h, the cooling bath was removed and the mixture was stirred at ambient temperature for 20 min. A solution of 2-chloro-6-(2-furyl)-9-(4-methoxyphenylmethyl)-9H-purine (Example 50) (248 mg, 0.73 mmol) in dry THF (8 mL) was added followed by a solution of tetrakis(triphenylphosphine)palladium(0) [generated in situ from tris(diphenylmethylideneacetone)dipalladium chloroform adduct (16 mg, 0.016 mmol) and triphenylphosphine 312 mg, 0.12 mmol)] in dry THF (4 mL). The resulting mixture was heated at reflux for 21 h and cooled to ambient temperature. Sat. aq. NH₄Cl (10 mL) was added and the aq. phase extracted with EtOAc (2×25 mL). The combined organic extracts were washed with brine (2×20 mL), dried (MgSO₄) and evaporated. The product was purified by flash chromatography on silica gel eluting with EtOAc-hexane (2:1); yield (154 mg) 66%, colorless crystals, mp 127-130° C. ¹H NMR (CDCl₃, 200 MHz): δ 2.85 (s, 3H, CH₃), 3.76 (s, 3H, OCH₃), 5.32 (s, 2H, CH₃), 6.61 (dd, J=3.6 and 1.8 Hz, 1H, H-4 in furyl), 6.85 (d, J=8.8 Hz, 2H, Ar), 7.23 (d, J=8.8 Hz, 2H, Ar), 7.72 (br d, J=3.6 Hz, 1H, H-3 in furyl), 7.81 (s, 1H, H-5 in furyl/H-8), 7.90 (s, 1H, H-5 in furyl/H-8); ¹³C N (CDCl3, 50 MHz): δ 26.3 (CH₃), 46.5 (CH₂), 55.3 (OCH₃), 112.4 (C-4 in furyl), 114.4 (CH in Ar), 117.1 (C-3 in furyl), 126.3 (C-5), 127.2 (C-1 in Ar), 129.3 (CH in Ar), 143.6 (C-8), 145.48 (C-5 in furyl), 145.54 (C-6), 149.7 (C-2 in furyl), 152.6 (C-4), 159.6 (C-4 in Ar), 162.4 (C-2); MS EI m/z (rel. %): 320 (47, M⁺), 305 (3), 122 (8), HRMS: Found 320.1287, calcd. for C₁₈H₁₆N₄O₂ 320.1273. Anal: Found: 67.29; H, 4.83; N 16.92. C₁₈H₁₆N₄O₃ requires C, 67.49; H, 5.03; N, 17.49%.

### EXAMPLE 77

### 2-Ethyl-6-(2-furyl)-9-(4-methoxyphenylmethyl)-9H-purine

ZnBr₂ (500 μL, 0.50 mmol, 1.00 M in THF) was added to [1,1-bis(diphenylphosphino)ferrocene]dichloro-palladium(II) complex with CH₂Cl₂ (1:1) (10 mg, 0.012 mmol). After cooling to −78° C., ethylmagnesium bromide (610 μL, 0.50 mmol, 0.82 M in THF) was added dropwise during 15 minutes, and the resulting mixture was stirred for an additional 15 minutes at −78° C. before a solution of 2-chloro-6-(2-furyl)-9-(4-methoxyphenylmethyl)-9H-purine (Example 50) (85 mg, 0.25 mmol) in THF (2.5 mL) was added. After further 10 minutes the mixture was allowed to reach ambient temperature and stirred over night. Sat. aq. NH₄Cl (10 mL) was added and the resulting mixture was extracted with EtOAc (3×15 mL). The organic phase was washed with water (15 mL) and brine (15 mL). The dried (MgSO₄) solution was evaporated together with a small amount of silica and added at the top of a silica gel column. The product was purified by flash chromatography eluting with CH₂Cl₂-acetone (19:1); yield 64 mg (65%) colorless crystalline solid, mp 119-120° C. ¹H NMR (CDCl₃, 300 MHz): δ 1.43 (t, J=7.6 Hz, 3H, CH₃), 3.13 (q, J=7.6 Hz, 2H, CH₂) 3.74 (s, 3H, OCH₃), 5.32 (s, 2H, NCH₃), 6.60 (dd, J=3.5 and 1.7 Hz, 1H, H-4 in furyl), 6.83 (d, J=8.7 Hz, 2H, Ar), 7.25 (d, J=8.7 Hz, 2H, Ar), 7.70-7.72 (m, 1H, H-5 in furyl), 7.82 (br d, J=3.5 Hz, 1H, H-3 in furyl), 7.93 (s, 1H, H-8); ¹³C NMR (CDCl₃, 75 MHz): δ 13.2 (CH₃), 32.6 (CH₂), 46.5 (NCH₂), 55.2 (OCH₃), 112.3 (C-4 in furyl), 114.3 (CH in Ar), 117.0 (C-3 in furyl), 126.3 (C-5), 127.3 (C-1 in Ar), 129.5 (CH in Ar), 143.6 (C-8), 145.4 (C-6), 145.5 (C-5 in furyl), 149.8 (C-2 in furyl), 152.6 (C-4), 159.6 (C-4 in Ar), 166.6 (C-2); MS EI m/z (rel. %): 334 (63, M⁺), 333 (10), 319 (4), 122 (9), 121 (100), 91 (3), 78 (5), 77 (5); HRMS: Found 334.1427, calcd. for C₁₂H₁₈N₄O₂ 334,1430; Anal. (C₁₉H₁₈N₄O₂) C, H, N.

### EXAMPLE 78

### 6-(2-Furyl)-9-(4-methoxyphenylmethyl)-2-(1-methylethyl)-9H-purine

The title compound was prepared by Negishi coupling between 1-methylethylzinc bromide (generated from 1-methylethylmagnesium bromide 327 μL, 0.50 mmol, 1.53 M in THF) and 2-chloro-6-(2-furyl)-9-(4-methoxyphenylmethyl)-9H-purine (Example 50) (85 mg, 0.25 mmol) as described for Example 77 above. The product was purified by flash chromatography on silica gel eluting with EtOAc-hexane (2:1); yield 66 mg (76%), off-white crystalline solid, mp. 123° C. ¹H NMR (CDCl₃, 300 MHz): δ 1.47 (d, J=6.9 Hz, 6H, 2×CH₃), 3.39 (m, J=6.9 Hz, 1H), 3.75 (s, 3H, OCH₃), 5.33 (s, 2H, NCH₂), 6.60 (dd, J=3.5 and 1.7 Hz, 1H, H-4 in furyl), 6.84 (d, J=8.7 Hz, 2H, Ar), 7.29 (d, J=8.7 Hz, 2H, Ar), 7.72 (dd, J=1.6 and 0.7 Hz, 1H, H-5 in furyl), 7.77 (dd, J=3.5 and 0.7 Hz, 1H, H-3 in furyl), 7.94 (s, 1H, H-8), ¹³C NMR (CDCl₃, 75 MHz): δ22.2 (CH₃), 37.6 (CH), 46.6 (NCH₂), 55.2 (OCH₃), 112.2 (C-4 in furyl), 114.3 (CH in Ar), 116.4 (C-3 in furyl), 126.5 (C-5), 127.5 (C-1 in Ar), 129.5 (CH in Ar), 143.6 (C-8), 145.5 (C-6), 145.6 (C-5 in furyl), 150.4 (C-2 in furyl), 152.7 (C-4), 159.6 (C-4 in Ar), 170.0 (C-2); MS EI m/z (rel. %): 348 (51, M⁻), 347 (5), 334 (5), 333 (31), 320 (7), 149 (3), 122 (5), 121 (100), 91 (2), 78 (2), 77 (4), 69 (5); HRMS: Found 348.1591, calcd. for C₂₀H₂₀N₄O₂ 348.1586; Anal. (C₂₀H₂₀N₄O₂) C, H, N.

### EXAMPLE 79

### 2-n-Butyl-6-(2-furyl)-9-(4-methoxyphenylmethyl)-9H-purine

The title compound was prepared by Negishi coupling between n-butylzinc bromide (generated from n-BuLi, 280 μL, 0.50 mmol, 1.80 M in hexane) and 2-chloro-6-(2-furyl)-9-(4-methoxyphenylmethyl)-9H-purine (Example 50) (85 mg, 0.25 mmol) as described for Example 77 above. The product was purified by flash chromatography on silica gel eluting with EtOAc-hexane (2:1); yield 39 mg (43%), colorless crystalline solid, mp 103° C. ¹H NMR (CDCl₃, 300 MHz): δ 0.96 (t, J=7.3 Hz, 3H, CH₃), 1.42-1.49 (m, 2H, CH₂) 1.85-1.93 (m, 2H, CH₂), 3.09-3.14 (m, 2H, CH₂), 3.76 (s, 3H, OCH₃), 5.33 (s, 2H, NCH₂), 6.61 (dd, J=3.5 and 1.7 Hz, 1H, H-4 in furyl), 6.85 (d, J=8.7 Hz, 2H, Ar), 7.26 (d, J=8.7 Hz, 2H, Ar), 7.72-7.74 (m, 1H, H-5 in furyl), 7.84 (br d, J=3.5 Hz, 1H, H-3 in furyl), 7.93 (r, 1H, H-8); ¹³C NMR (CDCl₃, 75 MHz): δ 14.0 (CH₃), 22.5 (CH₂), 31.2 (CH₂), 39.2 (CH₂), 46.6 (NCH₂), 55.2 (OCH₃), 112.4 (C-4 in furyl), 114.3 (CH in Ar), 117.0 (C-3 in furyl), 126.3 (C-5), 127.3 (C-1 in Ar), 129.5 (CH in Ar), 143.7 (C-8), 145.5 (C-6), 145.6 (C-5 in furyl), 149.8 (C-2 in furyl), 152.6 (C-4), 159.6 (C-4 in Ar), 165.9 (C-2); MS EI m/z (rel. %): 362 (7, M⁺), 347 (4), 334 (3), 333 (9), 321 (17), 320 (83), 122 (8), 121 (100), 91 (3), 78 (3), 77 (4); HRMS: Found 362.1735, calcd. for C₂₁H₂₂N₄O₂ 362,1743; Anal. (C₂₁H₂₂N₄O₂) C, H, N.

### EXAMPLE 80

### 2-Cyclopropyl-6-(2-furyl)-9-(4-methoxyphenylmethyl)-9H-purine

The title compound was prepared by Negishi coupling between cyclopropylzinc bromide (generated from cyclopropylmagnesium bromide 1.14 mL, 0.44 M, 0.50 mmol) and 2-chloro-6-(2-furyl)-9-(4-methoxyphenylmethyl)-9H-purine (Example 50) (85 mg, 0.25 mmol) as described for Example 77 above. The product was purified by flash chromatography on silica gel eluting with CH₂Cl₂-acetone (19:1); yield 62 mg (72%), colorless oil. Evaporation from acetone-hexane gave colorless crystalline compound, mp 115-116° C. ¹H NMR (CDCl₃, 300 MHz): δ 1.02-1.09 (m, 2H, CH₂), 1.18-1.25 (m, 2H, CH₂), 2.42 (tt, J=8.1 and 4.8 Hz, 1H, CH), 3.76 (s, 3H, OCH₃), 5.27 (s, 2H, NCH₂), 6.60 (dd, J=3.4 and 1.7 Hz, 1H, H-4 in furyl), 6.84 (d, J=8.7 Hz, 2H, Ar), 7.24 (d, J=8.7 Hz, 2H, Ar), 7.71-7.74 (m, 2H, H-5 and H-3 in furyl), 7.89 (s, 1H, H-8); ¹³C NMR (CDCl₃, 75 MHz): δ 10.7 (2×CH₂). 18.3 (CH), 46.5 (NCH₂), 55.2 (OCH₃), 112.3 (C-4 in furyl), 114.3 (CH in Ar), 116.4 (C-3 in furyl), 126.4 (C-5), 127.4 (C-1 in Ar), 129.5 (CH in Ar), 143.3 (C-8), 145.4 (C-6), 145.5 (C-5 in furyl), 150.3 (C-2 in furyl), 152.9 (C-4), 159.6 (C-4 in Ar), 166.5 (C-2); MS EI m/z (rel. %); 346 (78, M⁺), 345 (19), 331 (4), 239 (2), 225 (6), 122 (10), 121 (100), 91 (3), 78 (5), 77 (5); HRMS: Found 346.1422, calcd. for C₂₀H₁₀N₄O₂ 346.1430; Anal. (C₂₀H₁₈N₄O₂) C, H, N.

### EXAMPLE 81

### 2-Cyclopentenyl-6-(2-furyl)-9-(4-methoxyphenylmethyl)-9H-purine

A round bottom flask with condenser was charged with magnesium turnings (243 mg, 10.0 mmol) and a few crystals of I₂. 2 mL of a solution of cyclopentyl bromide (1.49 g, 10.0 mmol) in dry THF (10 mL) was added under stirring and gently heated to start the reaction. The rest of the solution was added in a rate to keep gentle reflux. The mixture was stirred for additional 2 h, cooled. and resulting cyclopentylmagnesium bromide was titrated. Cyclopentylmagnesium bromide (1.00 mL, 0.50 M, 0.50 mmol) was cooled to 0° C. and ZnBr₂ (500 μL, 1.00 M, 0.50 mmol) was added. The mixture was stirred 1 h at room temperature before a solution of 2-chloro-6-(2-furyl)-9-(4-methoxyphenylmethyl)-9H-purine (Example 50) (85 mg, 0.25 mmol) in dry THF (1.5 ML) and tetrakis (triphenylphosphine)palladium(0) [generated in situ from tris(dibenzylideneacetone)dipalladium (5.7 mg, 6.25 μmol) and triphenylphosphine (13.1 mg, 0.050 mmol)] in THF (1 mL) was added. The resulting mixture was then reflux for 21 h, cooled and worked up as described for Example 77 above. The product was purified by flash chromatography on silica gel eluting with CH₂Cl₂-acetone (19:1); yield 57 mg (61%), colorless oil. Evaporation from acetone-hexane gave colorless crystalline compound, mp 114-115° C. ¹H NMR (CDCl₃, 300 MHz): δ 1.63-1.79 (m, 2H, CH₂), 1.79-1.95 (m, 2H, CH₂), 1.95-2.08 (m, 2H, CH₂), 2.08-2.21 (m, 2H, CH₂), 3.77 (s, 3H, OCH₃), 5.33 (s, 2H, NCH₂), 6.61 (dd, J=3.5 and 1.8 Hz, 1H, H-4 in furyl), 6.85 (d, J=8.7 Hz, 2H, Ar), 7.28 (d, J=8.7 Hz, 2H, Ar), 7.73 (dd, J=1.8 and 0.7 Hz, H-5 in furyl), 7.76 (br d, J=3.5 Hz, H-3 in furyl), 7.94 (s, 1H, H-8); ¹³C NMR (CDCl₃, 75 MHz): δ 26.0 (2×CH₂), 33.5 (2×CH₂), 46.6 (NCH₂), 48.9 (CH), 55.3 (OCH₃), 112.3 (C-4 in furyl)₁ 114.4 (CH in Ar), 116.5 (C-3 in furyl), 126.5 (C-5), 127.6 (C-1 in Ar), 129.6 (CH in Ar), 143.6 (C-8), 145.5 (C-5 in furyl), 150.4 (C-2 in furyl), 152.8 (C-4), 159.7 (C-4 in Ar), 169.1 (C-2), C-6 was hidden; MS EI m/z (rel. %): 374 (44, M⁺), 373 (8), 347 (6), 346 (27), 334 (17), 333 (75), 253 (5), 225 (4), 122 (9), 121 (100), 91 (4), 78 (6), 77 (6); HRMS: Found 374.1732, calcd. for C₂₂H₂₂N₄O₂ 374.1743; Anal. (C₂₂H₂₂N₄O₂) C, H, N.

### EXAMPLE 82

### 2-Ethenyl-6-(2-furyl)-9-(4-methoxyphenylmethyl)-9H-purine

The title compound was prepared by Stille coupling between 2-chloro-6-(2-furyl-9-(4-methoxyphenylmethyl)-9H-purine (Example 50) (341 mg, 1.00 mmol) and ethenyl (tributyl)stannane (350 μL, 1.20 mmol) as described for (Example 57) (method A) above.The product was purified by flash chromatography on silica gel eluting with EtOAc-hexane (2:1); yield 254 mg (76%), colorless crystals, mp 94-95° C. ¹H NMR (CDCl₃, 300 MHz): δ 3.79 (s, 3H, OCH₃), 5.38 (s, 2H, NCH₂), 5.74 (dd, J=10.5 and 1.9 Hz, 1H, ═CH₂), 6.65 (dd, J=3.5 and 1.8 Hz, 1H, H-4 in furyl), 6.73 (dd, J=17.3 and 1.9 Hz, 1H, ═CH₂), 6.88 (d, J=8.7 Hz, 2H, Ar), 7.07 (dd, J=17.3 and 10.5 Hz, 1H, ═CH), 7.30 (d, J=8.7 Hz, 2H, Ar), 7.77 (dd, J=1.8 and 0.7 Hz, 1H, H-5 in furyl), 7.83 (dd, J=3.5 and 0.7 Hz, 1H, H-3 in furyl), 7.99 (s, 1H, H-8); ¹³C NMR (CDCl₃, 75 MHz): δ 46.6 (NCH₂), 55.2 (OCH₃), 112.4 (C-4 in furyl), 114.4 (CH in Ar), 116.8 (C-3 in furyl), 122.4 (═CH₂), 127.1 (C-5), 127.3 (C-1 Ar), 129.5 (CH in Ar), 136.9 (═CH), 144.4 (C-8), 145.6 (C-5 in furyl), 145.6 (C-6), 150.1 (C-2 in furyl), 152.7 (C-4), 158.9 (C-2), 159.7 (C-4 in Ar); MS EI m/z (rel. %): 332 (60, M⁺), 331 (5), 317 (3), 122 (9), 121 (100), 106 (3), 91 (3), 78 (6), 77 (6); HRMS: Found 332.1281, calcd. for C₁₉H₁₆N₄O₂ 332.1273; Anal. (C₁₉H₁₆N₄O₂) C, H, N.

### EXAMPLE 83

### 2-(1-Ethoxyvinyl)-6-(2-furyl)-9-(4-methoxyphenylmethyl)-9H-purine

The title compound was prepared by Stille coupling between 2-chloro-6-(2-furyl-9-(4-methoxyphenylmethyl)-9H-purine (Example 50) (170 mg, 0.50 mmol), and 1-(ethoxyvinyl)tributylstannan (203 μL, 0.60 mmol) as described for Example 57 (method A) above. The product was purified by flash chromatography on silica gel eluting with EtOAc-hexane (1:1); yield 155 mg (83%), colorless powdery crystals, mp 144-145° C. ¹H NMR (CDCl₃, 200 MHz): δ 1.53 (t, J=7.0 Hz, 3H, CH₃), 3.77 (s, 3H, OCH₃), 4.06 (q, J=7.0 Hz, 2H, OCH₂), 4.62 (s, 1H, ═CH₂), 5.38 (s, 2H, NCH₂), 5.77 2H, ═CH₂), 6.61 (dd, J=3.5 and 1.8 Hz, 1H, H-4 furyl), 6.86 (d, J=8.7 Hz, 2H, Ar), 7.29 (d, J=8.7 Hz, 2H, Ar), 7.73 (m, 1H, H-5 in furyl), 7.78 (dd, J≦3.5 and 0.7 Hz, 1H, H-3 in furyl), 7.96 (s, 1H, H-8); ¹³C NMR (CDC₃, 125 MHz): δ 14.4 (CH₃), 46.8 (NCH₂), 55.2 (OCH₃), 64.1 (OCH₂), 89.4 (═CH₂), 112.3 (C-4 in furyl), 114.4 (CH in Ar), 116.8 (C-3 in furyl), 127.0 (C-1 in Ar), 127.4 (C-5), 129.7 (CH in Ar), 144.6 (C-8), 145.6 (C-6), 145.7 (C-5 in furyl), 150.1 (C-2 in furyl), 152.6 (C-4), 156.2 (C-2), 157.9 (═CO), 159.7 (C-4 in Ar); MS EI m/z (rel. %): 376 (4, M⁻), 363 (2), 362 (16), 361 (69), 347 (3), 333 (4), 332 (15), 331 (15), 317 (5), 255 (2), 211 (1), 122 (8), 121 (100), 106 (1), 91 (3), 78 (4), 77 (5); HRMS: Found 376.1547, calcd. for C₂₁H₂₀N₄O₃ 376.1535; Anal. (C₂₁H₂₀N₄O₃) C, H, N.

### EXAMPLE 84

### 6-(2-Furyl)-9-(4-methoxyphenylmethyl)-2-phenyl-9H-purine

The title compound was prepared by Stille coupling between 2-chloro-6-(2-furyl-9-(4-methoxyphenylmethyl)-9H-purine (Example 50) (125 mg, 0.367 mmol), and trimethyl (phenyl)stannane (100 μl, 0.55 mmol) as described for Example 57 (method A) above. The product was purified by flash chromatography on silica gel eluting with EtOAc-hexane (1:3), followed by EtOAc-hexane (1:2) and EtOAc-hexane (1:1); yield 122 mg (87%), colorless crystalline compound, mp 140-142° C. ¹H NMR (CDCl₃, 300 MHz): δ 3.72 (s, 3H, OCH₃), 5.35 (s, 2H, NCH₂), 6.64 (dd, J=3.5 and 1.8 Hz, 1H, H-4 in furyl), 6.83 (d, J=8.7 Hz, 2H, Ar), 7.28 (d, J=8.7 Hz, 2H, Ar), 7.42-7.53 (m, 3H, Ph), 7.78 (dd, J=1.8 and 0.8 Hz, H-5 in furyl), 7.82 (dd, J=3.5 and 0.8 Hz, H-5 in furyl), 7.97 (s, 1H, H-8), 8.61-8.64 (m, 2H, Ph); ¹³C NMR (CDC₃, 75 MHz): δ 46.7 (NCH₂), 55.2 (OCH₃), 112.3 (C-4 in furyl), 114.4 (CH in Ar), 116.4 (C-3 in furyl), 127.0 (C-5), 127.5 (C-1 in Ar), 128.3 (CH in Ph), 128.4 (CH in Ph), 129.5 (C in Ar), 130.1 (CH in Ph), 138.1 (C-1 in Ph), 144.4 (C-8), 145.8 (C-5 in furyl), 145.8 (C-6), 150.8 (C-2 in furyl), 153.1 (C-4), 158.9 (C-2), 159.7 (C-4 in Ar); MS EI m/z (rel. %); 382 (70, M⁺), 381 (9), 261 (2), 367 (4), 122 (10), 121 (100), 106 (4), 91 (3), 78 (5), 77 (7); HRMS: Found 382.1430, calcd. for C₂₃H₁₈N₄O₂ 382.1430; Anal. (C₂₃H₁₈N₄O₂) C, H, N.

### EXAMPLE 85

### 2,6-(Di-2-furyl)-9-(4-methoxyphenylmethyl)-9H-purine

The title compound was prepared by Stille coupling between 2,6-dichloro-9-(4-methoxyphenylmethyl)-9H-purine (77 mg, 0.25 mmol), and 2-(tributylstannyl)furan (236 μl, 0.75 mmol) as described for Example 57 (method A) above. The product was purified by flash chromatography on silica gel eluting with EtOAc-hexane (1:1); yield 69 mg (74%), off-white powdery crystals, mp 155-156° C. ¹H NMR (CDCl₃, 300 MHz): δ 3.75 (s, 3H, OCH₃), 5.38 (s, 2H, CH₂), 6.56 (dd, J=3.4 and 1.8 Hz, 1H, H-4 in 2-furyl), 6.63 (dd, J=3.5 and 1.7 Hz, 1H, H-4 in 6-furyl), 6.85 (d, J=8.7 Hz, 2H, Ar), 7.27 (d, J=8.7 Hz, 2H, Ar), 7.39 (dd, J=3.4 and 0.7 Hz, 1H, H-3 in 2-furyl), 7.63-7.64 (m, H-5 in 2-furyl), 7.75-7.76 (m, H-5 in 6-furyl), 7.83 (dd, J=3.5 and 0.6 Hz, 1H, H-3 in 6-furyl), 7.94 (s, 1H, H-8); ¹³C NMR (CDCl₃, 75 MHz): δ 46.7 (CH₂), 55.2 (OCH₃), 112.0 (C-4 in 2-furyl), 112.4 (C-4 in 6-furyl), 112.7 (C-3 in 2-furyl), 114.4 (CH in Ar), 117.2 (C-3 in 6-furyl), 126.7 (C-5), 127.0 (C-1 in Ar), 129.6 (CH in Ar), 144.3 (C-8), 144.6 (C-5 in 2-furyl), 145.9 (C-5 in 6-furyl), 146.1 (C-2/C-4/C-6), 149.9 (C-2 in 6-furyl), 152.3 (C-2 in 2-furyl), 152.6 (C-2/C-4/C-6), 152.7 (C-4/C-2/C-6), 159.7 (C-4 in Ar); MS EI m/z (rel. %): 372 (55, M⁺), 371 (4), 357 (2), 251 (2), 122 (8), 121 (100), 106 (3), 91 (3), 78 (6), 77 (6); HRMS: Found 372.1214, calcd. for C₂₁H₁₆N₄O₃ 372.1222; Anal. (C₃₁H₁₆N₄O₃) C, H, N.

### EXAMPLE 86

### 2-Acetyl-6-(2-furyl)-9-(4-methoxyphenylmethyl)-9H-purine

2-(1-Ethoxyvinyl)-6-(2-furyl)-9-(4-methoxyphenylmethyl)-9H-purine (Example 83) (87 mg, 0.23 mmol) and acetone/1 M HCl (aq) 4:1 (2 mL) was stirred for 20 h at ambient temperature, before water (5 mL) was added and the mixture was extracted with EtOAc (4×15 mL). The combined organic extracts were washed with sat. aq. NAHCO₃ (10 mL), brine (10 mL), dried (MgSO₄) and evaporated. The product was purified by flash chromatography on silica gel eluting with EtOAc-hexane (2:1); yield 68 mg (85%), colorless powdery crystals, mp. 166-167° C. ¹H NMR (CDCl₃, 200 MHz): δ 2.87 (s, 3H, CH₃), 3.74 (s, 3H, OCH₃), 5.42 (s, 2H, CH₂), 6.63 (dd J=3.5 and 1.8 Hz, 1H, H-4 in furyl), 6.84 (d, J=8.6 Hz, 2H, Ar), 7.26 (d, J=8.6 Hz, 2H, Ar), 7.76 (dd J=1.6 and 0.7 Hz, 1H, H-5 in furyl), 7.81 (dd J=3.5 and 0.6 Hz, 1H, H-3 in furyl), 8.13 (s, 1H, H-8); ¹³C NMR (CDCl₃, 125 MHz): δ 27.2 (CH₃), 47.1 (CH₂), 55.2 (OCH₃), 112.6 (C-4 in furyl), 114.4 (CH in Ar), 117.6 (C-3 in furyl), 126.6 (C-1 in Ar), 128.9 (C-5), 129.7 (CH in Ar), 145.7 (C-6), 146.3 (C-5 in furyl), 146.6 (C-8), 149.7 (C-2 in furyl), 152.5 (C-4), 154.5 (C-2), 159.8 (C-4 in Ar), 197.7 (C═O); MS EI m/z (rel. %): 348 (55, M⁺), 347 (10), 333 (7), 305 (2), 122 (8), 121 (100), 91 (3), 78 (3), 77 (4); HRMS: Found 348.1208, calcd. for C₁₉H₁₆N₄O₃ 348.1222; Anal. (C₁₉H₁₆N₄O₃) C, H, N.

### EXAMPLE 87

### 6-(2-Furyl)-9-(4-methoxyphenylmethyl)-9H-purine-2-carbonitrile

To a mixture of zinc cyanide (70 mg, 0.60 mmol) in NMP (1 mL) was added 2-chloro-6-(2-furyl)-9-(4-methoxy-phenylmethyl)-9H-purine (Example 50) (170.4 mg, 0.50 mmol) in NMP (1 mL) and tetrakis(triphenylphosphine)palladium(0) [generated in situ from tris(diphenylmethylideneacetone) dipalladium (32 mg, 0.035 mmol) and triphenylphosphine (73 mg, 0.28 mmol)] in dry NMP (2 mL)]. The resulting mixture was stirred at 120° C. under N₂ for 24 h and cooled, before 2 M aq. ammonia (5 mL) was added. The mixture was extracted with EtOAc (3×20 mL), and the combined organic extracts were washed with 2 M aq. ammonia (15 mL) and brine (20 mL), dried (MgSO₄) and evaporated in vacuo. The product was isolated by flash chromatography on silica gel eluting with EtOAc-hexane (2:1); yield 80 mg (48%), colorless crystals, mp 205-207° C. ¹H NMR (CDCl₃, 500 MHz): δ 3.78 (s, 3H, OCH₃), 5.37 (s, 2H, CH₂), 6.67 (dd J=3.4 and 1.5 Hz, 1H, H-4 in furyl), 6.88 (d, J=8.6 Hz, 2H, Ar), 7.29 (d, J=8.6 Hz, 2H, Ar), 7.79 (br s, H-5 in furyl), 7.87 (br d J=3.5 Hz, 1H, H-3 in furyl), 8.17 (s, 1H, H-8); ¹³C NMR (CDCl₃, 125 MHz): δ; 47.5 (CH₂), 55.3 (OCH₃), 113.0 (C-4 in furyl), 114.6 (CH in Ar), 116.4 (CN), 119.2 (C-3 in furyl), 126.1 (C-1 in Ar), 129.2 (C-5), 129.8 (CH in Ar), 137.6 (C-6/C-2), 146.6 (C-2/C-6), 146.6 (C-8), 147.1 (C-5 in furyl), 148.6 (C-2 in furyl), 151.7 (C-4), 160.0 (C-4 in Ar); MS EI m/z (rel. %): 331 (26, M⁺), 122 (8), 121 (100), 106 (1), 91 (4), 89 (1), 78 (6), 77 (5); HRMS: Found 331.1056 calcd. for C₁₉H₁₃N₅O₂ 331.1069; Anal: Found: C, 64.62; H, 4.15; N, 20.76. C₁₉H₁₃N₅O₂ requires C, 65.25; H, 3.95; N, 21.14%.

### EXAMPLE 88

### 2-Trimethylsilylethynyl-6-(2-furyl)-9-(4-methoxyphenylmethyl)-9H-purine

A mixture of CuI (9.5 mg, 0.050 mmol), bis(triphenylphosphine)palladium(II) chloride (17.5 mg, 0.025 mmol), 2-chloro-6-(2-furyl)-9-(4-methoxyphenylmethyl)-9H-purine (Example 50) (170 mg, 0.50 mmol), triethylamine (250 μL, 1.79 mmol) and ethynyltrimethylsilane (139 μL, 1.00 mmol) in dry DMF (1.5 mL) was heated at 120° C. for 5 h in a sealed tube. After cooling the solution was evaporated in vacuo, dissolved in CH₂Cl₂ (20 mL), and filtered through a plug of silica (5 g) using CH₂Cl₂-acetone (7:3) (100 mL). The solution was evaporated in vacuo together with a small amount of silica and the product was purified by flash chromatography on silica gel eluting with CH₂Cl₂-acetone (39:1); yield 82 mg (41%), off-white crystalline comtound, mp 155-157° C. ¹H NMR (CDCl₃, 300 MHz): δ 0.26 (s, 9H, TMS), 3.71 (s, 3H, OCH₃), 5.31 (s, 2H, NCH₂), 6.58 (dd, J=3.5 and 1.7 Hz, 1H, H-4 in furyl), 6.80 (d, J=8.7 Hz, 2H, Ar), 7.17 (d, J=8.7 Hz, 2H, Ar), 7.69-7.70 (m, 1H, H-5 in furyl), 7.79 (br d, J=3.5 Hz, H-3 in furyl), 7.96 (s, 1H, H-8); ¹³C NMR (CDCl₃, 75 MHz): δ −0.4 (TMS), 46.6 (NCH₂), 55.2 (OCH₃), 92.1 (C≡), 103.2 (C≡), 112.5 (C-4 in furyl), 114.4 (CH in Ar), 117.8 (C-3 in furyl), 126.7 (C-1 in Ar), 127.4 (C-5), 129.4 (CH in Ar), 145.1 (C-8), 145.8 (C-6 /C-2), 146.0 (C-5 in furyl), 149.1 (C-2 in furyl), 152.0 (C-4), 159.7 (C-4 in Ar), C-2 or C-6 was hidden; MS EI m/z (rel. %): 402 (56, M⁺), 401 (8), 387 (9), 373 (1), 266 (1), 251 (3), 122 (9), 121 (100), 108 (2), 91 (3), 78 (4), 77 (4); HRMS: Found 402.1507, calcd. for C₂₂H₂₂N₄O₂Si 402.1512; Anal. (C₂₂H₂₂N₄O₂Si) C, H, N.

### EXAMPLE 89

### 2-Ethynyl-6-(2-furyl)-9-(4-methoxyphenylmethyl)-9H-purine

A mixture of 2-trimethylsilylethynyl-6-(2-furyl)-9-(4-methoxyphenylmethyl)-9H-purine (Example 88) (120 mg, 0.298 mmol) and K₂CO₃ (413 mg, 3.13 mmol) in CH₂Cl₂ (2.5 ml) and MeOH (2.5 mL) was stirred for 1.5 h at ambient temperature, diluted with CH₂Cl₂ (20 mL), washed with brine (3×15 mL), dried (MgSO₄) and evaporated in vacuo. The product was purified by flash chromatography on silica gel eluting with CH₂Cl₂-acetone (9:1); yield 72 mg (73%), off-white powdery crystals, mp 155° C. ¹H NMR (CDCl₃, 300 MHz): δ 3.09 (s, 1H, C≡CH), 3.75 (s, 3H, OCH₃), 5.31 (s, 2H, NCH₂), 6.62 (dd, J=3.5 Hz, J=1.7 Hz, 1H, H-4 in furyl), 6.85 (d, J=8.7 Hz, 2H, Ar), 7.24 (d, J=8.7 Hz, 2H, Ar), 7.73-7.75 (m, 1H, H-5 in furyl), 7.82 (br d, J=3.5 Hz, H-3 in furyl), 8.03 (s, 1H, H-8); ¹³C NMR (CDCl₃, 75 MHz): δ 46.8 (NCH₂), 55.3 (OCH₃ in Ar), 74.4 (C≡CH), 82.6 (C≡CH), 112.6 (C-4 in furyl), 114.5 (CH in Ar), 117.9 (C-3 in furyl), 126.8 (C-1 in Ar), 127.7 (C-5), 129.6 (CH in Ar), 145.2 (C-2), 145.3 (C-8), 146.2 (C-6), 146.3 (C-5 in furyl), 149.2 (C-2 in furyl), 152.1 (C-4), 159.8 (C-4 in Ar); MS EI m/z (rel. %): 330 (54, M⁺), 329 (4), 315 (3), 165 (1), 122 (9), 121 (100), 106 (3), 91 (4), 89 (1), 78 (6), 77 (6); HRMS: Found 330.1122, calcd. for C₁₉H₁₄N₄O₂ 330.1117; Anal. (C₂₂H₂₂N₄N₄O₂) C, H, N.

### EXAMPLE 90

### 6-Chloro-2-iodo-9-(4-methoxyphenylmethyl)-9H-purine

2-Amino-6-chloro-9-(4-methoxyphenylmethyl)-9H-purine (Example 58) (290 mg, 1.00 mmol), diiodomethane (10 mL) and isoamyl nitrite (2-7 mL, 20 mmol) was heated at 85° C. for 75 min. After cooling the solution was evaporated in vacuo, dissolved in EtOAc (30 mL), washed with brine (2×15 mL), dried (MgSO₄) and evaporated in vacuo together with a small amount of silica. The product was purified by flash chromatography on silica gel eluting with EtOAc-hexane (2:1); yield 197 mg (49%), colorless oil. ¹H NMR (CDCl₃, 300 MHz): δ 3.79 (s, 3H, OCH₃), 5.31 (s, 2H, CH₂), 6.89 (d, J=8.7 Hz, 2H, Ar), 7.25 (d, J=8.7 Hz, 2H, Ar), 7.90 (s, 1H, H-8); ¹³C NMR (CDCl₃, 75 MHz); δ 47.5 (CH₂), 55.2 (OCH₃), 114.4 (CH in Ar), 116.5 (C-2), 125.8 (C-1 in Ar), 129.6 (CH in Ar), 131.2 (C-5), 144.9 (C-8), 150.0 (C-6), 152.4 (C-4), 159.8 (C-4 in Ar); MS EI m/z (rel. %): 402/400 (16/46, M⁺), 399 (2), 136 (2), 122 (12), 121 (100), 91 (4), 89 (2), 78 (8), 77 (6); HRMS: Found 399.9584, calcd. for C₁₃H₁₀ClN₄O 399.9588; Anal. (C₁₃H₁₀CIN₄O) C, H, N.

### EXAMPLE 91

### 6-Chloro-9-(4-methoxyphenylmethyl)-2-(1-propynyl)-9H-purine

Propyne (1.06 g, 26.4 mmol) was condensed at −78° C. in a sealed tube. CuI (5.8 mg, 0.0305 mmol), bis(triphenylphosphine)palladiun(II) chloride (12.8 mg, 0.0183 mmol), a solution of 6-chloro-2-iodo-9-(4-methoxyphenyl)-9H-purine (Example 90) (122 mg, 0.305 mmol) in dry CH₃CN (2.2 mL), and triethylamine (1.10 mL, 7.89 mmol) was added. The tube was sealed and the mixture stirred at ambient temperature for 24 h. The reaction mixture was cooled to −78° C., opened, and allowed to reach room temperature under N₂. The mixture was evaporated in vacuo together with a small amount of silica and the product was purified by flash chromatography on silica gel eluting with EtOAc-hexane (1:1) followed by EtOAc-hexane (2:1) and EtOAc-hexane (4:1).

Yield 35 mg (37%), off-white crystalline compound, mp 143-145° C. ¹H NMR (CDCl₃, 300 MHz): δ 2.14 (s, 3H, CH₃), 3.79, (s, 3 E, OCH₃), 5.35 (s, 2H, CH₂), 6.88 (d, J=8.7 Hz, 2H, Ar), 7.25 (d, J=8.7 Hz, 2H, Ar), 8.00 (s, 1H, H-8); ¹³C NMR (CDCl₃, 75 MHz): δ 4.4 (CH₃), 47.3 (CH₂), 55.3 (OCH₃), 78.7 (C≡), 86.5 (C≡), 114.6 (CH in Ar), 126.2 (C-1 in Ar), 129.5 (CH in Ar), 130.5 (C-5), 145.4 (C-8), 145.9 (C-6/C-2), 150.8 (C-2/C-6), 151.8 (C-4), 160.0 (C-4 in Ar); MS EI m/z (rel. %): 312 isotop? (47, M⁺), 311 (5), 299 (1), 297 (3), 156 (1), 122 (14), 121 (100), 106 (1), 91 (5), 90 (1), 89 (2), 78 (8), 77 (7); HRMS: Found 312.0786, calcd. for C₁₆H₁₃N₄OCl 312.0778.

### EXAMPLE 92

### 6-(2-Furyl)-9-(4-methoxyphenylmethyl)-2-(1-propynyl)-9H-purine

Method A. The title compound was prepared by Stille coupling between 6-chloro-9-(4-methoxyphenylmethyl)-2-(1-propynyl)-9H-purine (Example 91) (29 mg, 0.093 mmol) and 2-(tributylstannyl)furan (35 μL, 0.11 mmol) as described for Example 57 (method A) above. The product was purified by flash chromatography on silica gel eluting with CH₃Cl₂-acetone (19:1); yield 18 mg (56%), off-white crystalline compound, mp 158-160° C. ¹H NMR (CDCl₃, 200 MHz): δ 2.12 (s, 3H, CH₃), 3.76 (s, 3H, OCH₃), 5.35 (s, 2H, NCH₂), 6.61 (dd, J=3.5 Hz, J=1.7 Hz, 1H, H-4 in furyl), 6.85 (d, J=8.7 Hz, 2H, Ar), 7.23 (d, J=8.7 Hz, 2H, Ar), 7.71-7.74 (m, 1H, H-5 in furyl), 7.82 (dd, J=3.5 and 0.6 Hz, H-3 in furyl), 7.96 (s, 1H, H-8); ¹³C NMR (CDCl₃, 75 MHz): δ 4.5 (CH₃), 46.6 (NCH₂), 55.2 (OCH₃), 79.7 (C≡), 84.6 (C≡), 112.5 (C-4 in furyl), 114.4 (CH in Ar), 117.6 (C-3 in furyl), 126.8 (C-1 in Ar), 127.1 (C-5), 129.5 (CH in Ar), 144.7 (C-8), 146.0 (C-5 in furyl), 146.1 (C-6/C-2), 146.6 (C-2/C-6), 149.2 (C-2 in furyl), 152.1 (C-4), 159.7 (C-4 in Ar); MS EI m/z (rel. %): 344 (100, M⁺), 343 (17), 330 (2), 329 (10), 315 (2), 122 (7), 121 (78), 91 (2), 78 (3), 77 (3); HRMS: Found 344.1280, calcd. for C₂₀H₁₆N₄O₂ 344.1273; Anal: Found: C, 67.99: H, 4.71; N, 15.75. C₂₀H₁₆N₄O₂ requires C, 69.76; H, 4.68; N, 16.27%. Method 1. Propyne (476 mg, 11.9 mmol) was condensed at −78° C. in a sealed tube. CuI (2.2 mg, 0.0116 mmol), bis(triphenylphosphine)palladium(II) chloride (4.9 mg, 0.007 mmol), a solution of 6-(2-furyl)-2-iodo-9-(4-methoxyphenylmethyl)-9H-purine (Example 62) (50 mg, 0.12 mmol) in dry CH₃CN (1 mL), and triethylamine (0.50 mL, 3.6 mmol) was added. The tube was sealed and the mixture was stirred at ambient temperature for 24 h. The reaction mixture was cooled to −78° C., opened, and allowed to reach ambient temperature under N₂. The mixture was evaporated in vacuo and the product was purified by flash chromatography on silica gel eluting with CH₂Cl₂-acetone (19:1); yield 18 mg (56%).

### EXAMPLE 93

### 6-Chloro-9-(4-methoxyphenylmethyl)-2-trifluoromethyl-9H-purine

Potassium carbonate (610 mg, 4.41 mmol) and 6-chloro-2-trifluoromethyl-9H-purine (328 mg, 1.47 mmol) in dry DMF (10 mL) was stirred at ambient temperature under N₂ for 20 minutes before 4-methoxyphenylmethyl chloride (400 μL, 2.95 mmol) was added. The resulting mixture was stirred for 16 h, filtered and evaporated in vacuo with a small amount of silica. The isomers were separated by flash chromatography using EtOAc-hexan (1:1) for elution. The yield of the title compound was 206 mg (41%) colorless crystalline compound, mp 104-106° C. ¹H NMR (CDCl₃, 300 MHz): δ 3.78 (s, 3H, OCH₃), 5.41 (s, 2H, CH₂), 6.88 (d, J=8.7 Hz, 2H, Ar), 7.30 (d, J=8.7 Hz, 2H, Ar), 8.18 (s, 1H, H-8); ¹³C NMR (CDCl₃, 75 MHz): δ 48.0 (CH₂), 55.3 (OCH₃), 114.7 (CH in Ar), 119.3 (q, J=275 Hz, CF₃), 125.7 (C-1 in Ar), 129.9 (CH in Ar), 132.6 (C-5), 146.9 (C-8), 149.8 (q, J=38 Hz, C-2), 151.6 (C-4/C-6), 151.7 (C-6/C-4), 160.1 (C-4 in Ar); ¹³F NMR (CDCl₃, 188 MHz): −69.20 (CF₃); MS EI m/z (rel. %): 344/342 (12/36, M⁺), 341 (2), 122 (9), 121 (100), 91 (3), 89 (2), 78 (7), 77 (6); HRMS: Found 342.0490, calcd. for C₁₄H₁₀ClF₃N₄O 342.0495; Anal: Found: C, 49.55; H, 3.23; N, 16.35 C₁₄H₁₀ClF₃N₄O requires C, 49.07; H, 2.94; N, 16.35.

### EXAMPLE 94

### 6-(2-Furyl)-9-(4-methoxyphenylmethyl)-2-trifluoromethyl-9H-purine

The title compound was prepared by stille coupling between 6-chloro-9-(4-methoxyphenylmethyl)-2-trifluoromethyl-9H-purine (Example 93) (130 mg, 0.38 mmol), and 2-(tributylstannyl)furan (143 μL, 0.46 mmol) as described for Example 57 (method A) above. The product was purified by flash chromatography on silica gel eluting with EtOAc-hexane (1:1) followed by EtOAc-hexane (2:1); yield 118 mg (83%), colorless crystals, mp 149-150° C. ¹H NMR (CDCl₃, 200 MHz): δ 3.78 (s, 3H, OCH₃), 5.41 (s, 2H, CH₂), 6.66 (dd, J=3.6 and 1.7 Hz, 1H, H-4 in furyl), 6.88 (d, J=8.6 Hz, 2H, Ar), 7.30 (d, J=8.6 Hz, 2H, Ar), 7.78-7.80 (m, 1H, H-5 in furyl), 7.89 (br d, J=3.5 Hz, 1H, H-3 in furyl), 8.13 (s, 1H, H-8); ¹³C NMR (CDCl₃, 75 MHz): δ 47.2 (CH₂), 55.3 (OCH₃), 112.8 (C-4 in furyl), 114.5 (CH in Ar), 118.7 (C-3 in furyl), 120.0 (q, J=275 Hz, CF₃), 126.4 (C-1 in Ar), 128.8 (C-5), 129.7 (CH in Ar), 146.1 (C-8), 146.2 (C-6), 146.8 (C-5 in furyl) 149.1 (C-2 furyl), 150.4 (q, J=36.5 Hz, C-2), 152.1 (C-4), 159.9 (C-4 in Ar); ¹⁹F NMR (CDCl₃, 188 MHz): −69.3 (CF₃). MS EI m/z (rel. %): 374 (40, M⁺), 187 (2), 122 (8), 121 (100), 91 (2), 78 (4); HRMS: Found 374.0986, calcd. for C₁₈H₁₃F₃N₄O₂ 374.0991; Anal. (C₁₈H₁₃F₃N₄O₂) C, H, N.

### EXAMPLE 95

### 6-Chloro-9-(4-methoxyphenylmethyl)-8-methyl-9H-purine

6-Chloro-9-(4-methoxyphenylmethyl)-9H-purine (Example 7) (137 mg, 0.50 mmol) in THF (2 mL) was added dropwise to a stirred solution of LDA [generated in situ from diisopropylamine (0.11 mL, 0.77 mmol) and n-BuLi (0.44 mL, 0.7 mmol, 1.6M in hexane)] in THF (2 mL) at −78° C. under N₂. After stirring for 1 h at −78° C., iodomethane (0.31 mL, 5.0 mmol) was added dropwise and the resulting mixture was stirred at −78° C. for 3.5 h, gradually warmed to ambient temperature over 2 h and stirred at ambient temperature for 15 h. Sat. aq. NH₄Cl (15 mL) was added and the mixture as extracted with EtOAc (2×25 mL). The combined organic extracts were washed with brine (20 mL), dried (MgSO₄) and evaporated in vacuo. The product was purified by flash chromatography on silica gel eluting with EtOAc-hexane (2:1); yield 96 mg (67%), colorless crystals, mp 121-123° C. ¹H NMR (CDCl₃, 200 MHz): δ 2.59 (s, 3H, CH₃), 3.76 (s, 3H, OCH₃), 5.36 (s, 2H, CH₂), 6.83 (d, J=8.6 Hz, 2H, Ar), 7.12 (d, J=8.6 Hz, 2H, Ar), 8.70 (s, 1H, H-2); MS EI m/z (rel. %): 290/288 (10/30, M⁺), 275 (1), 273 (4), 246 (1), 122 (9), 121 (100), 91 (3), 78 (5), 77 (5); HRMS: Found 288.0776, calcd. for C₁₄H₁₃ClN₄O 288.0778. Anal. Found: 58.36; H, 4.44. C₁₄H₁₃ClN₄O requires C, 58.82; H, 4.54%.

### EXAMPLE 96

### 6,8-Dichloro-9-(4-methoxyphenylmethyl)-9H-purine

6-Chloro-9-(4-methoxyphenylmethyl)-9H-purine (Example 7) (275 mg, 1.00 mmol) in THF (4 mL) was added dropwise to a stirred solution of LDA [generated in situ from diisopropylamine (0.21 mL, 1.50 mmol) and n-BuLi (0.88 mL, 1.40 mmol, 1.6M in hexane)] in THF (4 mL) at −78° C. under N₂. After stirring for 1 h at −78° C., a solution of hexachloroethane (473 mg, 2.00 mmol) in THF (4 mL) was added dropwise and the resulting mixture was stirred at −78° C. for 2 h, and 10 min without cooling Sat. aq. NH₄Cl (15 mL) was added and the mixture as extracted with EtOAc (2×25 mL). The combined organic extracts were washed with brine (20 mL), dried (MgSO₄) and evaporated in vacuo. The product was purified by flash chromatography on silica gel eluting with EtOAc-hexane (1:1) followed by EtOAc-hexane (1:3); yield 251 mg (81%), colorless crystals, mp 110-112° C. ¹H NMR (CDCl₃, 200 MHz): δ 3.73 (s, 3H, OCH₃), 5.38 (s, 2H, CH₂), 6.81 (d, J=8.5 Hz, 2H, Ar), 7.30 (d, J=8.5 Hz, 2H, Ar), 8.72 (s, 1H, H-2); ¹³C NMR (CDCl₃, 50 MHz): δ 47.1 (CH₂) 55.3 (OCH₃), 114.3 (CH in Ar), 126.0 (C-1 in Ar), 129.6 (CH in Ar), 130.5 (C-5), 144.1 (C-8), 149.4 (C-6), 152.0 (C-2), 152.4 (C-4), 159.7 (C-4 in Ar); MS EI m/z (rel. %): 312/310/308 (1/10/16, M⁺), 293 (1), 273 (2), 122 (8), 121 (100), 91 (3), 78 (5), 77 (5), HRMS: Found 308.0246, calcd. for C₁₃H₁₀Cl₂N₄O 308.0232. Anal. (C₁₃H₁₀ClN₄O) C, H, N.

### EXAMPLE 97

### 8-Bromo-6-chloro-9-(4-methoxyphenylmethyl)-9H-purine

6-Chloro-9-(4-methoxyphenylmethyl)-9H-purine (Example 7) (272 mg, 0.99 mmol) in THF (4 mL) was added dropwise to a stirred solution of LDA [generated in situ from diisopropylamine (0.21 mL, 1.50 mmol) and n-BuLi (0.88 mL, 1.40 mmol, 1.6M in hexane)] in THF (4 mL) at −78° C. under N₂. After stirring for 1 h at −78° C., a solution of 1,2-dibromo-1,1,2,2,-tetrachloroethane (651 mg, 2.00 mmol) in THF (4 mL) was added dropwise and the resulting mixture was stirred at −78° C. for 5 h, gradually warmed to ambient temperature over 2 h and stirred at ambient temperature for 14 h. Sat. aq. NH₄Cl (15 ml) was added and the mixture as extracted with EtOAc (2×25 mL). The combined organic extracts were washed with brine (20 mL), dried (MgSO₄) and evaporated in vacuo. The product was purified by flash chromatography on silica gel eluting with EtOAc-hexane (1:2); yield 293 mg (83%), colorless wax. ¹H NMR (CDCl₃, 200 MHz): δ 3.76 (s, 3H, OCH₃), 5.41 (s, 2H, CH₂), 6.83 (d, J=8.4 Hz, 2H, Ar), 7.33 (d, J=8.4 Hz, 2H, Ar), 8.74 (s, 1H, H-2); ¹³C NMR (CDCl₃, 50 MHz): δ 47.8 (CH₂), 55.1 (OCH₃), 114.2 (CH in Ar), 126.0 (C-1 in Ar), 129.4 (CH in Ar), 131.6 (C-5), 133.9 (C-8), 149.1 (C-6), 151.8 (C-2), 152.6 (C-4), 159.5 (C-4 in Ar); MS EI m/z (rel. %): 356/354/352 (3/13/10, M⁺), 275 (3), 273 (11), 122 (8), 121 (100), 91 (3), 78 (6), 77 (6); HRMS: Found 351.9731, calcd. for C₁₃H₁₀BrClN₄O 351.9727; Anal. (C₁₃H₁₀BrClN₄O) C, H, N.

### EXAMPLE 98

### 6-(2-Furyl)-9-(4-methoxyphenylmethyl)-8-methyl-9H-purine

Method A: LDA [generated from diisopropylamine (0.085 mL, 0.60 mmol) and n-BuLi (0.34 mL, 0.55 mmol, 1.6M in hexane)] in THF (2 mL) was added dropwise to a stirred solution of 6-(2-furyl)-9-(4-methoxyphenylmethyl)-9H-purine (Example 33) (153 mg, 0.5 mmol) in THF (10 mL) at −78° C. under N₂. After stirring for 50 min at −78° C., iodomethane (0.0.62 mL, 1.0 mmol) was added dropwise and the resulting mixture was stirred at −78° C. for 5 h, gradually warmed to ambient temperature over ca. 2 h and stirred at ambient temperature for ca. 15 h. Sat. aq. NH₄Cl (15 mL) was added and the mixture as extracted with EtOAc (3×25 mL). The combined organic extracts were washed with brine (2×20 mL), dried (MgSO₄) and evaporated in vacuo. The product was purified by flash chromatography on silica gel eluting with EtOAc-hexane (2:1); yield (110 mg) 69%, colorless wax. ¹H NMR (CDCl₃, 200 MHz): δ 2.56 (s, 3H, CH₃), 3.72 (s, 3H, OCH₃), 5.34 (s, 2H, CH₂), 6.61 (dd, J=3.4 and 1.8 Hz, 1H, H-4 in furyl), 6.79 (d, J=8.6 Hz, 2H, Ar), 7.09 (d, J=8.6 Hz, 2H, Ar), 7.72 (br s, 1H, H-5 in furyl), 7.76 (br d, J=3.4 Hz, 1H, H-3 in furyl), 8.88 (s, 1H, H-2); ¹³C NMR (CDCl₃, 50 MHz): δ 14.9 (CH₃), 45.4 (CH₂), 55.2 (OCH₃), 112.4 (C-4 in furyl), 114.3 (CH in Ar), 116.5 (C-3 in furyl), 127.3 (C-1 in Ar), 127.6 (C-5), 128.4 (CH in Ar), 145.4 (C-5 in furyl), 145.7 (C-6), 149.9 (C-2 in furyl), 151.8 (C-2), 153.5 (C-4/C-8), 154.3 (C-4/C-8), 159.3 (C-4 in Ar); MS EI m/z (rel. %): 320 (77, M⁺), 305 (8), 213 (1), 160 (3), 122 (9), 121 (100), 91 4), 78 (6), 77 (6); HRMS: Found 320.1276, calcd. for C₁₈H₁₆N₄O₂ 320.1273; Found: 67.30; H, 5.02; N 17.26. C₁₈H₁₆N₄O₂ requires C, 67.75; H, 5.22; N, 17.49%.

Method B: The title compound was prepared by Stille coupling on 6-chloro-9-(4-methoxyphenylmethyl)-8-methyl-9H-purine (Example 95) (0.65 mmol) as described for Example 57 above and the product was purified by flash chromatography on silica gel eluting with EtOAc-hexane (2:1); yield 176 mg (85%).

### EXAMPLE 99

### 8-Chloro-6-(2-furyl)-9-(4-methoxyphenylmethyl)-9H-purine

Method A: LDA [generated from diisopropylamine (0.13 mL, 0.89 mmol) and n-BuLi (0.51 mL, 0.81 mmol, 1.6 M in hexane)] in THF (4 mL) was added dropwise to a stirred solution of 6-(2-furyl)-9-(4-methoxyphenylmethyl)-9H-purine (Example 33) (227 mg, 0.74 mmol) in THF (10 mL) at −78° C. under N₂. After stirring for 55 min at −78° C., hexachloroethane (350 mg, 1.48 mmol) in THF (4 mL) was added dropwise and the resulting mixture was stirred at −78° C. for 3 h and without cooling for 15 min. Sat. aq. NH₄Cl (15 mL) was added and the mixture as extracted with EtOAc (3×25 mL). The combined organic extracts were washed with brine (2×20 mL), dried (MgSO₄) and evaporated in vacuo. The product was purified by flash chromatography on silica gel eluting with EtOAc-hexane (1:5) followed by EtOAc-hexane (1:2); yield 137 mg (54%), colorless small needles, mp 156-157° C. ¹H NMR (CDCl₃, 200 MHz): δ 3.76 (s, 3H, OCH₃), 5.41 (s, 2H, CH₂), 6.64 (dd, J=3.4 and 1.8 Hz, 1H, H-4 in furyl), 6.84 (d, J=8.4 Hz, 2H, Ar), 7.33 (d, J=8.4 Hz, 2H, Ar), 7.74 (br s, 1H, H-5 in furyl), 7.80 (br d, J=3.4 Hz, 1H, H-3 in furyl), 8.94 (s, 1H, H-2); ¹³C NMR (CDCl₃, 50 MHz): δ 46.4 (CH₂), 55.3 (OCH₃), 112.6 (C-4 in furyl), 114.2 (CH in Ar), 117.6 (C-3 in furyl), 126.6 (C-1 in Ar), 127.2 (C-5), 129.5 (CH in Ar), 143.2 (C-6/C-8), 144.5 (C-6/C-8), 145.9 (C-5 in furyl), 149.0 (C-2 in furyl), 152.7 (C-2), 153.1 (C-4), 159.6 (C-4 in Ar); MS EI m/z (rel. %): 342/340 (10/30, M⁺), 170 (1), 122 (8), 121 (100), 91 (3), 78 (4), 77 (5); HRMS: Found 340.1717, calcd. for C₁₇R₁₃ClN₄O₂ 340.0727; Anal. (C₁₇H₁₃ClN₄O₂): C, H, N. 8-Chloro-6-(5-chloro-2-furyl)-9-(4-methoxyphenylmethyl)-9H-purine (Example 100) 41 mg (15%) was also isolated (data see below).

Method B: A mixture of tris (diphenylmethylideneacetone) dipalladium chloroform adduct (23 mg, 0.022 mmol) and tri(2-furyl)phosphine (37 mg, 0.16 mmol) in DMF (8 mL) was stirred at ambient temperature under N₂ atm. for 15 min., before 6,8-dichloro-9-(4-methoxyphenylmethyl)-9H-purine (Example 96) (228 mg, 0.74 mmol) and 2-furyl(tributyl)tin (0.28 mL, 0.88 mmol) were added. The resulting mixture was stirred for 18 h at 50° C. and evaporated in vacuo. The residue was dissolved in acetonitrile (30 mL) and washed with hexane (15×10 mL). The acetonitrile layer was evaporated and the product was isolated by flash chromatography on silica eluting with EtOAc-hexane (1:5) followed by EtOAc-hexane (1:2); yield 118 mg (47%), colorless small needles. Fractions containing 6,8-di-(2-furyl)-9-(4-methoxyphenylmethyl)-9H-purine (Example 103) (see below) were also isolated.

### EXAMPLE 100

### 8-Chloro-6-(5-chloro-2-furyl)-9-(4-methoxyphenylmethyl)-9H-purine

Colorless microcrystaline solid, mp 166-168° C. ¹H NMR (CDCl₃, 200 MHz): δ 3.76 (s, 3H, OCH₃), 5.40 (s, 2H, CH₂), 6.43 (d, J=3.6 Hz, 1H, H-4 in furyl), 6.83 (d, J=8.6 Hz, 2H, Ar), 7.33 (d, J=8.6 Hz, 2H, Ar), 7.82 (d, J=3.6 Hz, 1H, H-3 in furyl), 8.94 (s, 1H, H-2); ¹³C NMR (CDCl₃, 50 MHz): δ 46.5 (CH₂), 55.3 (OCH₃), 109.7 (C-4 in furyl), 114.2 (CH in Ar), 120.0 (C-3 in furyl), 126.6 (C-1 in Ar), 127.0 (C-5), 129.5 (CH in Ar), 141.0 (C-6/C-8/C-5 in furyl), 143.28 (C-6/C-8/C-5 in furyl), 143.32 (C-6/C-8/C-5 in furyl), 148.1 (C-2 in furyl), 152.6 (C-4), 152.7 (C-2), 159.6 (C-4 in Ar); MS EI m/z (rel. %): 378/376/374 (1/10/15, M⁺), 122 (8), 121 (100), 91 (3), 89 (1), 87 (2), 78 (5), 77 (5); HRMS: Found 374.0343, calcd. for C₁₇H₁₂Cl₂N₄O₂ 374.0337; Anal. (C₁₇H₁₂Cl₂N₄O₂): C, H, N.

### EXAMPLE 101

### 8-Bromo-6-(2-furyl)-9-(4-methoxyphenylmethyl)-9H-purine

LDA [generated from diisopropylamine (0.17 mL, 1.2 mmol) and n-BuLi (0.69 mL, 1.1 mmol, 1.6 M in hexane)] in THF (4 mL) was added dropwise to a stirred solution of 6-(2-furyl)-9-(4-methoxyphenylmethyl)-9H-purine (Example 33) (306 mg, 1.00 mmol) in THF (20 mL) at −78° C. under N₂. After stirring for 50 min at −78° C., a solution of 1,2-dibromo-1,1,2,2,-tetrachloroethane (651 mg, 2.00 mmol) in THF (2 mL) was added dropwise and the resulting mixture was stirred at −78° C. for 5 h, gradually warmed to ambient temperature over ca. 4 h and stirred at ambient temperature for ca. 14 h. Sat. aq. NH₄Cl (15 mL) was added and the mixture as extracted with EtOAc (3×25 mL). The combined organic extracts were washed with brine (2×20 mL), dried (MgSO₄) and evaporated in vacuo. The product was purified by flash chromatography on silica gel eluting with EtOAc-hexane (1:5) followed by EtOAc-hexane (1:1); yield (153 mg) 45%, colorless crystals, mp 150-151° C. ¹H NMR (CDCl₃, 200 MHz): δ 3.74 (s, 3H, OCH₃), 5.39 (s, 2H, CH₂), 6.62 (dd, J=3.4 and 1.8 Hz, 1H, H-4 in furyl), 6.81 (d, J=8.6 Hz, 2H, Ar), 7.30 (d, J=8.6 Hz, 2H, Ar), 7.68 (br s, 1H, H-5 in furyl), 7.72 (br d, J=3.4 Hz, 1H, H-3 in furyl), 8.81 (s, 1H, H-2); ¹³C NMR (CDCl₃, 50 MHz): δ 47.2 (CH₂), 55.3 (OCH₃), 112.6 (C-4 in furyl), 114.2 (CH in Ar), 117.8 (C-3 in furyl), 126.8 (C-1 in Ar), 128.5 (C-5), 129.4 (CH in Ar), 133.0 (C-8), 144.4 (C-6), 145.9 (C-5 in furyl), 149.0 (C-2 in furyl), 152.7 (C-2), 153.0 (C-4), 159.5 (C-4 in Ar); MS EI m/z (rel. %): 386/384 (15/15, M⁺), 305 (6), 122 (8), 121 (100), 91 (3), 78 (5), 77 (5) ; HRMS: Found 384.0220, calcd. for C₁₇H₁₃BrN₄O₂ 3840222; Anal: Found: 53.25; H, 3.47; N 14.04. C₁₇H₁₃BrN₄O₂ requires C, 53.01; H, 3.40; N, 14.54%.

### EXAMPLE 102

### 9-(4-Methoxyphenylmethyl)-8-methyl-(5-methyl-2-furyl)-9H-purine

To a stirred solution of LDA [generated from diisopropylamine (0.25 mL, 1.74 mmol) and n-BuLi (0.91 mL, 1.45 mmol, 1.6 M in hexane)] in THF (4 mL) at −78° C. under N₂, was added dropwise a solution of 6-(2-furyl)-9-(4-methoxyphenylmethyl)-9H-purine (Example 33) (177 mg, 0.58 mmol) in THF (12 mL). After stirring for 1 h at −78° C., iodomethane (0.36 mL, 5.8 mmol) was added dropwise and the resulting mixture was stirred at −78° C. for 6 h, gradually warmed to ambient temperature over ca. 2 h and stirred at ambient temperature for ca. 15 h. Sat. aq. NH₄Cl (15 mL) was added and the mixture as extracted with EtOAc (3×25 mL). The combined organic extracts were washed with brine (2×20 mL), dried (MgSO₄) and evaporated in vacuo. The product was purified by flash chromatography on silica gel eluting with EtOAc-hexane (1:1); yield 106 mg (55%), off-white wax. ¹H NMR (CDCl₃, 200 MHz): δ 2.44 (s, 3H, CH₃), 2.50 (s, 3H, CH₃), 3.68 (s, 3H, OCH₃), 5.28 (s, 2H, CH₂), 6.20 (d, J=3.4 Hz, 1H, H-4 in furyl), 6.75 (d, J=8.6 Hz, 2H, Ar), 7.06 (d, J=8.6 Hz, 2H, Ar), 7.73 (d, J=3.4 Hz, 1H, H-3 in furyl), 8.84 (s, 1H, H-2); ¹³C NMR (CDCl₃, 125 MHz): δ 14.0 (CH₃), 14.7 (CH₃), 45.2 (CH₂), 55.1 (OCH₃), 109.2 (C-4 in furyl), 114.2 (CH in Ar), 118.9 (C-3 in furyl), 127.1 (C-5), 127.3 (C-1 in Ar), 128.4 (CH in Ar), 144.0 (C-6), 147.9 (C-2 in furyl), 151.8 (C-2), 153.0 (C-4), 153.8 (C-8), 156.0 (C-5 in furyl), 159.3 (C-4 in Ar); MS EI m/z (rel. %): 334 (53, M⁺), 319 (5), 291 (1), 277 (2), 213 (1), 167 (1), 122 (8), 121 (100), 91 (3), 78 (4), 77 (5); HRMS: Found 334.1434, calcd. for C₁₉H₁₈N₄O₂ 324.1430.

### EXAMPLE 103

### 6,8-Di(2-furyl)-9-(4-methoxyphenylmethyl)-9H-purine

Fractions containing a mixture of the title compound and 8-chloro-6-(2-furyl)-9-(4-methoxyphenylmethyl)-9H-purine (Example 99, method B, see above) were subjected to flash chromatography on silica gel eluting with acetone-hexane (1:4); yield 65 mg (24%), off-white crystalline solid, mp 134-136° C. ¹H NMR (CDCl₃, 200 MHz): δ 3.72 (s, 3H, OCH₃), 5.76 (s, 2H, CH₂), 6.60 (dd, J=3.6 and 1.8 Hz, 1H, H-4 in furyl), 6.65 (dd, J=3.4 and 1.6 Hz, 1H, H-4 in furyl), 6.77 (d, J=8.6 Hz, 2H, Ar), 7.16 (d, J=8.6 Hz, 2H, Ar), 7.27 (br d, J=3.6 Hz, 1H, H-3 in furyl), 7.66 (m, 1H, H-5 in furyl), 7.74 (m, 1H, H-5 in furyl), 7.94 (br d, J=3.4 Hz, 1H, H-3 in furyl), 8.94 (s, 1H, H-2); ¹³C NMR (CDCl₃, 50 MHz): δ 46.5 (CH₂), 55.2 (OCH₃), 112.4 (C-4 in furyl), 112.6 (C-4 in furyl), 114.1 (CH in Ar), 114.9 (C-3 in furyl), 117.6 (C-3 in furyl), 128.1 (C-1 in Ar and C-5), 128.4 (CH in Ar), 144.7 (C-8), 144.8 (C-5 in furyl), 145.5 (C-6 and C-5 in furyl), 149.4 (2×C-2 in furyl), 152.4 (C-2), 153.5 (C-4), 159.1 (C-4 in Ar); MS EI m/z (rel. %): 372 (43, M⁺), 265 (1), 122 (8), 121 (100), 119 (3), 91 (3), 78 (5), 77 (6); HRMS: Found 372.1215, calcd. for C₂₃H₁₆N₄O₃ 372.1222; Anal. (C₂₁H₁₆N₄O₃) C, H, N.

### EXAMPLE 104

### 6-(2-Furyl)-8-methoxy-9-(4-methoxyphenylmethyl)-9H-purine

A mixture of 8-chloro-6-(2-furyl)-9-(4-methoxyphenylmethyl)-9H-purine (Example 99) (205 mg, 0.60 mmol) in a 0.7 M solution of sodium methoxide in methanol (10 mL) was stirred at reflux under N₂-atm. for 4 h, cooled and poured into a beaker containing cold sat. aq. NH₄Cl (10 mL). Most of the methanol was removed by evaporation in vacuo and the aqueous mixture was extracted with EtOAc (4×20 mL). The combined organic layers were dried (MgSO₄) and evaporation in vacuo. The product was purified by flash chromatography on silica gel eluting with acetone-hexane (2:7); 111 mg (55%), colorless crystals. 6-(2-Furyl)-8-hydroxy-9-(4-methoxyphenylmethyl)-9H-purine (Example 105) 54 mg (28%) was also isolated (data see below). Mp 143-144° C. ¹H NMR (CDCl₃, 200 MHz); δ 3.75 (s, 3H, OCH₃), 4.28 (s, 3H, OCH₃), 5.18 (s, 2H, CH₂), 6.61 (dd, J=3.4 and 1.8 Hz, 1H, H-4 in furyl), 6.82 (d, J=8.6 Hz, 2H, Ar), 7.31 (d, J=8.6 Hz, 2H, Ar), 7.72 (br s, 1H, H-5 in furyl), 7.81 (br d, J=3.4 Hz, 1H, H-3 in furyl), 8.81 (s, 1H, H-2); ¹³C NMR (CDCl₃, 50 MHz): δ 44.2 (CH₂), 55.2 (OCH₃), 57.5 (OCH₃), 112.2 (C-4 in furyl), 114.0 (CH in Ar), 115.9 (C-3 in furyl), 126.1 (C-5), 127.7 (C-1 in Ar), 129.3 (CH in Ar), 141.3 (C-6), 145.6 (C-5 in furyl), 149.4 (C-2 in furyl), 150.7 (C-2), 152.3 (C-4), 158.3 (C-8), 159.2 (C-4 in Ar); MS EI m/z (rel. %); 336 (34, M⁺), 168 (1), 122 (8), 121 (100), 91 (3), 78 (4), 77 (5); HRMS: Found 336.1221, calcd. for C₁₈H₁₆N₄O₃ 336.1222; Anal. (C₁₈H₁₆N₄O₃) C, H, N.

### EXAMPLE 105

### 6-(2-Furyl)-8-hydroxy-9-(4-methoxyphenylmethyl)-9H-purine

Colorless crystals, mp 249-251° C., ¹NMR (CDCl₃, 200 MHz): δ 3.75 (s, 3H, OCH₃), 5.07 (s, 2H, CH₂), 6.61 (dd, J=3.2 and 1.8 Hz, 1H, H-4 in furyl), 6.83 (d, J=8.8 Hz, 2H, Ar), 7.28 (br d, J=3.2 Hz, 1H, H-3 in furyl), 7.45 (d, J=8.8 Hz, 2H, Ar), 7.64 (br s, 1H, H-5 in furyl), 8.65 (s, 1H, H-2), 8.97 (br s, 1H, NH); ¹H NMR (DMSO-d₆, 600 MHz); δ 3.70 (s, 3H, OCH₃), 4.96 (s, 2H, CH₂), 6.73 (m, 1H, H-4 in furyl), 6.88 (d, J=8.4 Hz, 2H, Ar), 7.29-7.31 (m 3H, H-3 in furyl and Ar), 7.91 (s, 1H, H-5 in furyl), 8.55 (s, 1H, H-2), 11.66 (br s, 1H, NH); ¹³C NMR (DMSO-d₆, 150 MHz): δ 42.1 (CH₂), 55.0 (OCH₃), 111.9 (C-3 in furyl), 112.4 (C-4 in furyl), 113.9 (CH in Ar), 115.3 (C-5), 128.4 (C-1 in Ar), 129.0 (CH in Ar), 132.3 (C-6),145.5 (C-5 in furyl), 149.6 (C-2 in furyl), 150.4 (C-2), 150.5 (C-4), 153.4 (C-8), 158.7 (C-4 in Ar); MS EI m/z (rel. %): 322 (35, M⁺), 122 (4), 121 (100), 78 (1), 77 (2); HRMS: Found 322.1067, calcd. for C₁₇H₁₄N₄O₃ 322.1066; Anal. (C₁₇H₁₄N₄O₃) C, H, N.

### EXAMPLE 106

### 7-Chloro-[3-(4-methoxybenzylamino)]-3H-[1,2,3]triazolo[4,5-d]pyrimidine

A mixture of 5-Amino-4-chloro-6-(4-methoxybenzylamino) pyrimidine (670 mg, 2.53 mmol), AcOH (50% in water, 12 mL) and NaNO₂ (192 mg, 2.78 mg) in dichloromethane (12 mL) was stirred at ambient temperature for 30 min, dilluted with dichloromethane (20 mL), washed with water (10 mL) and brine (10 mL), dried (MgSO₄) and evaporated in vacou. The products was purified by flash chromatography on silica gel eluting with dichoromethane; yield 562 mg (81%), m.p. 110-112° C., off-white powdery crystals. ¹H NMR (CDCl₃ 200 MHz) δ 3.75 (s, 3H, OCH₃), 5.81 (s, 2H, CH₂), 6.85 (d, J=8.4 Hz, 2H, Ar), 7.42 (d, J=8.4 Hz, 2H, Ar), 8.90 (s, 1H, H-5); ¹³C NMR (CDCl₃, 50 MHz) δ 51.0 (CH₂), 55.3 (OCH₃), 114.3 (CH in Ar), 125.8 (C-1 in Ar), 130.0 (CH in Ar), 134.1 (C-7a), 149.4 (C-3a/C-7), 154.0 (C-3a/C-7), 155.3 (C-5), 159.9 (C-4 in Ar); MS EI m/z (rel. %): 277/275 (12/36, M⁺), 248 (39), 246 (92), 234 (6), 232 (18), 218 (7), 216 (22), 134 (13), 122 (9), 121 (199); HRMS: Found 275.0582, calcd. for C₁₂H₁₀ClN₅O 275.0574.

### EXAMPLE 107

### 7-(2-Furyl)-[3-(4-methoxybenzylamino)]-3H-[1,2,3]triazolo[4,5-d]pyrimidine

A mixture of 7-chloro-[3-(4-methoxybenzylamino)]-3H-[1,2,3]triazolo[4,5-d]pyrimidine (Example 106) (278 mg, 1.0 mmol), 2-furyl(tributyl)tin (0.48 mL, 1.5 mmol) and (Ph₃P)₂PdCl₂ (36 mg. 0.05 mmol) in DMF (4 mL) was stirred at 90° C. under N₂-atm for 4 h, and evaporated in vacuo. KF in MeOH (sat. sol., 10 mL) was added to the resudue and the mixt. stirred for 18 h. The product was purified by flash chromatography on silica gel eluting with EtOAc-hexane (1:2) followed by EtOAc-hexane (1:1) and finally pure EtOAc; yield 275 mg (90%), m.p. 166-168° C., colorless powdery crystals. ¹H NMR (CDCl₃, 200 MHz) δ 3.75 (s, 3H, OCH₃), 5.81 (s, 2H, CH₂), 6.72 (dd, J 3,6 and 1.8 Hz, 1H, furyl), 6.84 (d, J=8.6 Hz, 2H, Ar), 7.43 (d, J=8.6 Hz, 2H, Ar), 7.82 (br s, 1H, furyl),) 8.11 (br d, J=3.6 Hz, 1H, furyl), 9.07 (s, 1H, H-5); ¹³C NMR (CDCl₃, 50 MHz) δ 50.1 (CH₂), 55.0 (OCH₃), 113.1 (CH in furyl), 114.0 (CH in Ar), 120.3 (CH in furyl), 126.3 (C-1 in Ar), 129.7 (CH in Ar), 130.3 (C-7a), 147.2 (CH in furyl) 147.7 (C-3a/C-7/C-2 in furyl), 148.4 (C-3a/C-7/C-2 in furyl) 149.1 (C-3a/C-7/C-2 in furyl), 155.9 (C-5), 159.5 (C-4 in Ar); MS EI m/z (rel. %): 307 (30, M⁺), 279 (26), 278 (82), 264 (15), 250 (17), 159 (7), 146 (9), 121 (100); HRMS: Found 307.1065, calcd. for C₁₆H₁₃N₅O₂ 307.1069.

### EXAMPLE 108

### 4-Chloro-7-(4-methoxybenzyl)-7H-pyrrolo[2,3-d]pyrimidine

A mixture of 7-deazapurine (487 mg, 3.17 mmol) and K2CO3 (1.13 g, 9.51 mmol) in DMF (12 mL) was stirred at ambient temperature under N2-atm, for 45 min, before p-methoxybenzyl chloride (0.86 ml, 6.34 mmol) was added. The resulting reaction mixture was stirred at ambent temperature for 23 h, filtered and evaporated in vacuo. The product was purified by flash chromatography on silica gel eluting with EtOAc-hexane (1:4); yield 522 mg (60%); colorless wax. ¹H NMR (CDCl₃, 200 MHz) δ 3.76 (s, 3H, OCH₃), 5.36 (s, 2H, CH₂), 6.57 (d, J=3.8 Hz, 1H, H-5), 6.83 (d, J=8.6 Hz, 2H, Ar), 7.14-7.19 (m, 3H, Ar and H-6), 8.65 (s, 1H, H-2); ¹³C NMR (CDCl₃, 50 MHz) δ 47.6 (CH₂), 54.9 (CH₃), 99.3 (C-5), 113.9 (CH in Ar), 117.0 (C-4a), 127.9 (C-1 in Ar), 128.7 (C-6), 128.8 (CH in Ar), 150.3 (C-2), 150.5 (C-4/C-7a), 151.5 (C-4/C-7a), 159.0 (C-4 in Ar); MS EI m/z (rel. %): 275/273 (14/38, M⁺), 154 (14), 122 (16), 121 (100), 91 (6), 90 (2), 98 (3), 78 (13), 77 (9); HRMS: Found 273.0668, calcd. for C₁₄H₁₂ClN₃O 273.0669.

### EXAMPLE 109

### 4-(2-Furyl)-7-(4-methoxybenzyl)-7H-pyrrolo[2,3-d]pyrimidine

A mixture of 4-chloro-7-(4-methoxybenzyl)-7H-pyrrolo[2,3-d]pyrimidine (Example 108) (277 mg, 1.01 mmol), 2-furyl(tributyl)tin (0.48 mL, 1.5 mmol) and (Ph₃P)₂PdCl₂ (36 mg. 0.05 mmol) in DMF (4 mL) was stirred at 90° C. under N₂-atm for 17 h, and evaporated in vacuo. KF in MeOH (sat. sol., 10 mL) was added to the resudue and the mixt. stirred for 18 h. The product was purified by flash chromatography on silica gel eluting with EtOAc-hexane (2:3); yield 280 mg (92%); m.p. 130-131° C., colorless small needles. ¹H NMR (CDCl₃, 200 MHz) δ 3.76 (s, 3H, OCH₃), 5.39 (s, 2H, CH₂), 6.61 (dd, J 3.2 and 1.6 Hz, 1H, furyl), 6.83 (d, J=8.6 Hz, 2H, Ar), 6.99 (d, J=3.6 Hz, 1H, H-5), 7.14-7.19 (m, 3H, Ar and H-6), 7.38 (br d, J 3.2 Hz, 1H, furyl), 7.68 (br s, 1H, furyl), 8.87 (s, 1H, H-2); ¹³C NMR (CDCl₃, 50 MHz) δ 47.3 (CH₂), 55.2 (CH₃), 101.1 (C-5), 112.1 (Ch in furyl), 112.6 (CH in furyl), 112.8 (C-4a), 114.1 (CH in Ar), 128.5 (C-6), 128.7 (C-1 in Ar), 128.9 (CH in Ar), 144.8 (CH in furyl), 147.1 (C in furyl), 151.3 (C-2), 151.8 (C-4/C-7a), 153.2 (C-4/C-7a), 159.02(C-4 in Ar); MS EI m/z (rel. %): 305 (59, M⁺), 290 (3), 198 (2), 185 (7), 184 (4), 157 (4), 156 (3), 153 (2), 129 (4), 122 (14), 121 (100); HRMS: Found 305.1160, calcd. for C₁₈H₁₅N₃O₂ 305.1164.

### EXAMPLE 110

### 2-Ethylthio-6-(2-furyl)-9-(4-methoxyphenylmethyl)-9H-purine

A mixture of KCN (65 mg, 1.0 mmol) and 6-(2-furyl)-9-(4-methoxyphenylmethyl)-2-nitro-9H-purine (Example 72) (88 mg, 0.25 mmol) in ethanethiol (10 mL) and CH₂C₂ (5 mL) was heated at reflux for 40 h, cooled to ambient temperature and evaporated in vacuo. The product was purified by flash chromatography on silica gel eluting with EtOAc-hexane (2:1); yield 61 mg (67%), colorless crystalline compound, mp 116-117° C. ¹H NMR (CDCl₃, 300 MHz) δ 1.43 (t, J=7.3 Hz, 3H, CH₃), 3.25 (q, J=7.3 Hz, 2H, SCH₂), 3.76 (s, 3H, OCH₃ in Ar), 5.28 (s, 2H, NCH₂), 6.60 (dd, J=3.5 Hz, J=1.5 Hz, 1H, H-4 in furyl), 6.84 (d, J=8.7 Hz, 2H, Ar), 7.24 (d, J=8.7 Hz, 2H, Ar), 7.71-7.73 (m, 2H, H-3 and H-5 in furyl), 7.88 (s, 1H, H-8) ; ¹³C NMR (CDCl₃, 75 MHz) δ 14.6 (CH₃), 25.7 (SCH₂), 46.7 (NCH₂), 55.3 (OCH₃), 112.3 (C-4 in furyl), 114.3 (CH in Ar), 116.9 (C-3 in furyl), 125.6 (C-5), 127.2 (C-1 in Ar), 129.4 (CH in Ar), 143.0 (C-8), 146.0 (C-5 in furyl), 150.0 (C-2 in furyl), 153.0 (C-4), 159.6 (C-4 in Ar), 165.6 (C-2), C-6 was hidden; MS EI m/z (rel. %) 366 (71, M⁺), 351 (3), 333 (8), 305 (4), 246 (4), 245 (30), 122 (9), 121 (100), 78 (4), 77 (4); HRMS Found 366.1139, calcd. for C₁₉H₁₉N₄O₂S 366.1150.

### EXAMPLE 111

### 6-(2-Furyl)-9-(4-methoxyphenylmethyl)-2-propoxy-9H-purine

A mixture of 6-(2-furyl)-9-(4-methoxyphenylmethyl)-2-nitro-9H-purine (Example 72) (88 mg, 0.25 mmol) and KF (56 mg, 1.0 mmol) in n-propanol (10 mL) was heated at reflux for 6 h. The solution was cooled to ambient temperature and evaporated in vacuo. The product was purified by flash chromatography on silica gel eluting with CH₂Cl₂-acetone (19:1); yield 67 mg (74%), colorless oil. Evaporation from acetone-hexane gave colorless crystals, mp 119° C. ¹H NMR (CDCl₃, 300 MHz) δ 1.06 (t, J=7.4 Hz, 3H, CH₃), 1.81-1.93 (m, 2H, CH₂), 3.75 (s, 3H, OCH₃), 4.41 (t, J=6.8 Hz, 2H, OCH₂), 5.26 (s, 2H, NCH₂), 6.59 (dd, J=3.4 and 1.8 Hz, 1H, H-4 in furyl), 6.84 (d, J=8.8 Hz, 2H, Ar), 7.23 (d, J=8.8 Hz, 2H, Ar), 7.71-7.73 (m, 2H, H-3 and H-5 in furyl), 7.83 (s, 1H, H-8); ¹³C NMR (CDCl₃, 75 MHz) δ 10.6 (CH₃), 22.2 (CH₂), 46.5 (NCH₂), 55.3 (OCH₃), 69.5 (OCH₂), 112.3 (C-4 in furyl), 114.3 (CH in Ar), 116.7 (C-3 in furyl), 124.5 (C-5), 127.3 (C-1 in Ar), 129.4 (CH in Ar), 142.9 (C-8), 145.8 (C-5 in furyl), 147.1 (C-6), 150.2 (C-2 in furyl), 154.3 (C-4), 159.6 (C-4 in Ar), 161.9 (C-2); MS EI m/z (rel. %) 364 (27, M⁺), 335 (9), 334 (3), 322 (14), 321 (4), 122 (9), 121 (100), 91 (3), 78 (4), 77 (5); HRMS Found 364.1531, calcd. for C₂₀H₂₀N₄O₃ 364.1535.

### EXAMPLE 112

### 6-(2-Furyl)-9-(4-methoxyphenylmethyl)-2-isopropoxy-9H-purine

A mixture of 6-(2-furyl)-9-(4-methoxyphenylmethyl)-2-nitro-9H-purine (Example 72) (88 mg, 0.25 mmol) and KCN (65 mg, 1.0 mmol) in isopropanol (10 mL) was heated at reflux for 48 h and cooled to ambient temperature. CH₂Cl₂ (50 mL), was added and the mixture was washed with water (3×25 mL) and brine (25 mL), dried (MgSO₄) and evaporated in vacuo. The product was purified by flash chromatography on silica gel eluting with CH₂Cl₂-acetone (19:1); yield 48 mg (53%), off-white crystalline compound, mp 145-146° C. ¹H NMR (CDCl₃, 300 MHz) δ 1.42 (d, J=6.2 Hz, 6H, 2×CH₃), 3.75 (s, 3H, OCH₃), 5.24 (s, 2H, NCH₂), 5.40 [sept, J=6.2 Hz, 1H, CH(CH₃)₃], 6.61 (dd, J=3.4 and 1.8 Hz, 1H, H-4 in furyl), 6.83 (d, J=8.7 Hz, 2H, Ar), 7.23 (d, J=8.7 Hz, 2H, Ar), 7.70-7.71 (m, 2H, H-3 and H-5 in furyl), 7.82 (s, 1H, H-8); ¹³C NMR (CDCl₃, 75 MHz) δ 21.9 (2×CH₃), 46.5 (NCH₂), 55.2 (OCH₃), 70.4 [CH(CH₃)₂], 112.2 (C-4 in furyl), 114.3 (CH in Ar), 116.5 (C-3 in furyl), 124.3 (C-5), 127.3 (C-1 in Ar), 129.4 (CH in Ar), 142.8 (C-8), 145.8 (C-5 in furyl), 147.2 (C-6), 150.3 (C-2 in furyl), 154.3 (C-4), 159.6 (C-4 in Ar), 161.3 (C-2); MS EI m/z (rel. %) 364 (26, M⁺), 350 (3), 349 (14), 322 (12), 321 (4), 306 (9), 122 (9), 121 (100), 78 (4), 77 (5); HRMS Found 364.1530, calcd. for C₂₀H₂₀N₄O₃ 364.1535

### EXAMPLE 113

### 2-Butoxy-6-(2-Furyl)-9-(4-methoxyphenylmethyl)-9H-purine

A mixture of 6-(2-furyl)-9-(4-methoxyphenylmethyl)-2-nitro-9H-purine (Example 72) (88 mg, 0.25 mmol) and KF (56 mg, 1.0 mmol) in n-butanol (10 mL) was heated at reflux for 7 h. The solution was cooled to ambient temperature and evaporated in vacuo with a small amount of silica gel. The product was purified by flash chromatography on silica gel eluting with CH₂Cl₂-acetone (19:1); yield 77 mg (81%), colorless crystals, mp 113° C. ¹H NMR (CDCl₃, 300 MHz) δ 0.94 (t, J=7.4 Hz, 3H, CH₃), 1.43-1.56 (m, 2H, CH₂), 1.76-1.85 (m, 2H, CH₂), 3.72 (s, 3H, OCH₃), 4.43 (t, J=6.7 Hz, 2H, OCH₂), 5.22 (s, 2H, NCH₂), 6.56 (dd, J=3.5 and 1.8 Hz, 1H, H-4 in furyl), 6.80 (d, J=8.7 Hz, 2H, Ar), 7.20 (d, J=8.7 Hz, 2H, Ar), 7.68-7.71 (m, 2H, H-3 and H-5 in furyl), 7.81 (s, 1H, H-8); ¹³C NMR (CDCl₃, 75 MHz) δ 13.8 (CH₃), 19.1 (CH₂), 30.9 (CH₂), 46.4 (NCH₂), 55.1 (OCH₃), 67.6 (OCH₂), 112.2 (C-4 in furyl), 114.2 (CH in Ar), 116.6 (C-3 in furyl), 124.4 (C-5), 127.3 (C-1 in Ar), 129.3 (CH in Ar), 142.9 (C-8), 145.7 (C-5 in furyl), 147.0 (C-6), 150.1 (C-2 in furyl), 154.2 (C-4), 159.5 (C-4 in Ar), 161.8 (C-2); MS EI m/z (rel. %) 378 (21, M⁺), 349 (6), 348 (6), 335 (6), 324 (5), 322 (16), 321 (4), 122 (9), 121 (100), 77 (4); HRMS Found 378.1688, calcd. for C₂₁H₂₂N₄O₃ 378.1692.

### EXAMPLE 114

### 2-Benzyloxy-6-(2-Furyl)-9-(4-methoxyphenylmethyl)-9H-purine

A mixture of 6-(2-furyl)-9-(4-methoxyphenylmethyl)-2-nitro-9H-purine (Example 72) (88 mg, 0.25 mmol) and KF (56 mg, 1.0 mmol) in benzyl alcohol (10 mL) was heated at 100° C. for 2.5 h. The solution was cooled to ambient temperature and CH₂Cl₂ (50 mL) was added to the residue. The solution was washed with water (4×25 mL), brine (2×25 mL), dried (MgSO₄) and evaporated in vacuo. The product was purified by flash chromatography on silica gel eluting with CH₂Cl₂-acetone (19:1); yield 73 mg (71%), colorless crystalline compound, mp 122° C. ¹H NMR (CDCl₃, 300 MHz) δ 3.74 (s, 3H, OCH₃), 5.24 (s, 2H, NCH₂), 5.54 (s, 2H, OCH₂), 6.60 (dd, J=3.5 and 1.8 Hz, 1H, H-4 in furyl), 6.81 (d, J=8.7 Hz, 2H, Ar), 7.19 (d, J=8.7 Hz, 2H, Ar), 7.27-7.36 (m, 3H, H-3 and H-4 in Ph), 7.50-7.55 (m, 2H, H-2 in Ph), 7.71-7.75 (m, 2H, H-3 and H-5 in furyl), 7.84 (s, 1H, H-8); ¹³C NMR (CDCl₃, 75 MHz) δ 46.5 (NCH₂), 55.2 (OCH₃), 69.4 (OCH₂), 112.3 (C-4 in furyl), 114.3 (CH in Ar), 116.8 (C-3 in furyl), 124.7 (C-5), 127.3 (C-1 in Ar), 127.8 (CH in Ph), 128.3 (CH in Ph), 129.3 (CH in Ar), 136.9 (C-1 in Ph), 143.1 (C-8), 145.9 (C-5 in furyl), 147.1 (C-6), 150.1 (C-2 in furyl), 154.2 (C-4), 159.6 (C-4 in Ar), 161.4 (C-2); MS EI m/z (rel. %) 412 (68, M⁺), 335 (9), 306 (22), 305 (8), 292 (9), 291 (47), 122 (8), 121 (100), 91 (28), 77 (6); HRMS Found 412.1541, calcd. for C₂₄H₂₀N₄O₂ 412.1535.

### EXAMPLE 115

### 6-(2-Furyl)-9-(4-methoxyphenylmethyl)-2-phenoxy-9H-purine

A mixture of 6-(2-furyl)-9-(4-methoxyphenylmethyl)-2-nitro-9H-purine (Example 72) (88 mg, 0.25 mmol), KF (56 mg, 1.0 mmol) and phenol (10.7 g) was heated at 100° C. for 21 h. The mixture was cooled to ambient temperature and CH₂Cl₂ (50 mL) was added to the residue. The solution was washed with sat. aq. Na₂CO₃ (4×25 mL), water (25 mL), brine (2×25 mL), dried (MgSO₄), and evaporated in vacuo. The residue was dissolved in CH₂Cl₂ (50 mL), washed with aq. NaOH (4×25 mL, pH=10), water (25 mL) and brine (25 mL), dried (MgSO₄), and evaporated in vacuo. The product was purified by flash chromatography on silica gel eluting with CH₂Cl₂ followed by CH₂Cl₂-acetone (19:1) and CH₂Cl₂-acetone (9:1); yield 68 mg (68%), colorless crystalline compound. ¹H NMR (CDCl₃, 300 MHz) δ 3.73 (s, 3H, OCH₃), 5.11 (s, 2H, NCH₂), 6.56 (dd, J=3.5 and 1.7 Hz, 1H, H-4 in furyl), 6.78 (d, J=8.8 Hz, 2H, Ar), 7.14 (d, J=8.8 Hz, 2H, Ar), 7.19-7.28 (m, 3H, Ph), 7.36-7.43 (m, 2H, Ph), 7.65 (dd, J=3.5 and 0.8 Hz, 1H, H-3 in furyl), 7.69 (dd, J=1.7 and 0.8 Hz, 1H, H-5 in furyl), 7.89 (s, 1H, H-8); ¹³C NMR (CDCl₃, 75 MHz) δ 46.8 (NCH₂), 55.2 (OCH₃), 112.4 (C-4 in furyl), 114.2 (CH in Ar), 117.1 (C-3 in furyl), 121.5 (CH in Ph), 124.7 (CH in Ph), 125.2 (C-5), 126.9 (C-1 in Ar), 129.1 (CH in Ph), 129.7 (CH in Ar), 143.5 (C-8), 146.1 (C-5 in furyl), 147.3 (C-6), 149.9 (C-2 in furyl), 153.5 (C-1 in Ph), 154.0 (C-4), 159.6 (C-4 in Ar), 161.1 (C-2); MS EI m/z (rel. %) 398 (52, M⁺), 397 (3), 381 (1), 278 (2), 277 (7), 122 (8), 121 (100), 91 (4), 78 (6), 77 (10); HRMS Found 398.1375, calcd. for C₂₃H₁₈N₄O₃ 398.1379; Anal; Found: C, 69.49; H, 4.67; N, 14.58. C₂₃H₁₈N₄O₃ requires C, 69.34; H, 4.55; N, 14.06.

### EXAMPLE 116

### Supplementary Material—Elemental Analysis of Novel Compounds

6-Chloro-2-fluoro-9-(4-methoxyphenylmethyl)-9H-purine (a)

Anal: Found: C, 53.61; H, 3.48; N, 19.28. C₁₃H₁₀ClFN₄O requires C, 53.35; H, 3.44; N, 19.14.

6-Chloro-2-fluoro-7-(4-methoxyphenylmethyl)-7H-purine (b)

Anal: Found: C, 53.78; H, 3.56; N, 19.15. C₁₃H₁₀ClFN₄O requires C, 53.35; H, 3.44; N, 19.14.

6-Chloro-2-iodo-9-(4-methoxyphenylmethyl)-9H-purine.

Anal: Found: C, 39.07; H, 2.60; N, 13.91. C₁₃H₁₀CIN₄O requires C, 38.98; H, 2.52; N, 13.99.

6-Chloro-9-(4-methoxyphenylmethyl)-2-(4-methoxyphenylmethylamino)-9H-purine

Anal: Found: 61.41; H, 4.83; N, 17.00. C₂₁H₂₀ClN₅O₂ requires C, 61.54; H, 4.92; N, 17.09.

6-Chloro-9-(4-methoxyphenylmethyl)-2-[di(4-methoxyphenylmethyl)amino]-9H-purine

Anal: Found: 65.51; H, 5.48; N, 13.00. C₂₉H₂₈ClN₅O₃ requires C, 65.72; H, 5.32; N, 13.21.

6-Chloro-9-(4-methoxyphenylmethyl)-2-nitro-9H-purine.

Anal: Found: C, 48.85; H, 3.17; N, 21.97. C₁₃H₁₀ClN₅O₃ requires C, 48.84; H, 3.15; N, 21.91.

6-Chloro-9-(4-methoxyphenylmethyl)-2-trifluoromethyl-9H-purine (a).

Anal: Found: C, 49.55; H, 3.23; N, 16.35. C₁₄H₁₀ClF₃N₄O requires C, 49.07; H, 2.94; N, 16.35.

6-Chloro-7-(4-methoxyphenylmethyl)-2-trifluoromethyl-7H-purine (b).

Anal: Found: C, 49.317 H, 3.34; N, 16.44. C₁₄H₁₀ClF₃N₄O requires C, 49.07; H, 2.94; N, 16.35.

2-Fluoro-6-(2-furyl)-9-(4-methoxyphenylmethyl)-9H-purine.

Anal: Found: C, 62.83; H, 4.15, N, 16.90. C₁₇H₁₃FN₄O₂ requires C, 62.96; H, 4.04; N, 17.28.

6-(2-Furyl)-9-iodo-9-(4-methoxyphenylmethyl)-9H-purine.

Anal: Found: C, 47.29; H, 3.15; N, 12.89. C₁₇H₁₃IN₄O₂ requires C, 47.24; H, 3.03; N, 12.96.

6-(2-Furyl)-9-(4-methoxyphenylmethyl)-2-(N-methylamino)-9H-purine.

Anal: Found: C, 64.487 H, 4.97; N, 21.27. C₁₈H₁₇N₅O₂ requires C, 64.47; H, 5.11; N, 20.88.

N-[6-(2-Furyl)-9-(4-methoxybenzyl)-9H-purin-2-yl]acetamide

Anal: Found: C, 62.65; H, 4.74; N, 19.13. C₁₉H₁₇N₅O₃ requires C, 62.80; H, 4.72; N, 19.27.

6-(2-Furyl)-2-(4-methoxyphenylmethylamino)-9-(4-methoxyphenylmethyl)-9H-purine.

Anal: Found: 68.02; H, 5.28; N, 15.09. C₁₇H₁₅N₅O₂ requires C, 68.04; H, 5.25; N, 15.86.

2-(N,N-Dimethylamino-6-(2-furyl)-9-(4-methoxyphenylmethyl)-9H-purine.

Anal: Found: C, 65.28; H, 5.41; N, 20.05. C₂₁H₁₉N₅O₄ requires C, 65.32; H, 5.48; N, 20.04.

6-(2-Furyl)-9-(4-methoxyphenylmethyl)-2-nitro-9H-purine.

Anal: Found: C, 58.05; H, 3.61; N, 19.84, C₁₇H₁₃N₅O₄ requires C, 58.12; H, 3.73; N, 19.93.

6-(2-Furyl) -2-methoxy-9-(4-methoxyphenylmethyl)-9H-purine.

Anal: Found: 64.06; H, 4.73; N, 16.27. C₁₈H₁₆N₄O₃ requires C, 64.28; H, 4.80; N, 16.66.

6-(2-Furyl)-9-(4-methoxyphenylmethyl)-2-methylthio-9H-purine.

Anal: Found: C, 61.30; H, 4.56; N, 15.86. C₁₈H₁₆N₄O₂S requires C, 61.35; H, 4.58; N, 15.90.

6-(2-Furyl)-9-(4-methoxyphenylmethyl)-2-methylsulfinyl-9H-purine.

Anal: Found: C, 57.10; H, 4.55; N, 15.05. C₁₈H₁₆N₄O₃S requires C, 58.68; H, 4.38; N, 15.21.

6-(2-Furyl)-9-(4-methoxyphenylmethyl)-2-methylsulfonyl-9H-purine.

Anal: Found: C, 56.32; H, 4.26; N, 14.33. C₁₈H₁₆N₄O₄S requires C, 56.24; H, 4.20; N, 14.57%.

6-(2-Furyl)-2-methyl-9-(4-methoxyphenylmethyl)-9H-purine.

Anal: Found: 67.29; H, 4.83; N, 16.92. C₁₈H₁₆N₄O₃ requires C, 67.49; H, 5.03; N, 17.49.

2-Ethyl-6-(2-furyl)-9-(4-methoxyphenylmethyl)-9H-purine.

Anal: Found: C, 68.08; H, 5.39; N, 16.55. C₁₉H₁₆N₄O₂ requires C, 68.25; H, 5.43; N, 16.76.

6-(2-Furyl)-9-(4-methoxyphenylmethyl)-2-(1-methylethyl)-9H-purine.

Anal: Found: C, 68.82; H, 5.77; N, 16.14. C₂₀H₂₀N₄O₂ requires C, 68.95; H, 5.79; N, 16.08.

2-Butyl-6-(2-furyl)-9-(4-methoxyphenylmethyl)-9H-purine.

Anal: Found: C, 70.01; H, 6.25; N, 15.52. C₂₁H₂₂N₄O₂ requires C, 69.59; H, 6.12; N, 15.46.

2-Cyclopropyl-6-(2-furyl)-9-(4-methoxyphenylmethyl)-9H-purine.

Anal: Found: C, 69.37; H, 5.18; N, 16.12. C₂₀H₁₈N₄O₂ requires C, 69.35; H, 5.24; N, 16.17.

2-Cyclopentenyl-6-(2-furyl)-9-(4-methoxyphenylmethyl)-9H-purine.

Anal: Found: C, 70.53; H, 5.98; N, 15.01. C₂₂H₂₂N₄O₂ requires C, 70.57; H, 5.92; N, 14.96.

6-(2-Furyl)-9-(4-methoxyphenylmethyl)-2-trifluoromethyl-9H-purine.

Anal: Found: C, 57.97; H, 3.82; N, 15.15. C₁₈H₁₃F₃N₄O₂ requires C, 57.76; H, 3.50; N, 14.97.

2-Ethenyl-6-(2-furyl)-9-(4-methoxyphenylmethyl)-9H-purine.

Found: C, 69.01; H, 4.84; N, 16.67. C₁₉H₁₆N₄O₂ requires C, 68.66; H, 4.85; N, 16.86.

2-(1-Ethoxyvinyl)-6-(2-furyl)-9-(4-methoxyphenylmethyl)-9H-purine.

Anal: Found: C, 67.16; H, 5.50; N, 14.78. C₂₁H₂₀N₄O₃ requires C, 67.01; H, 5.36; N, 14.88%.

2-Acetyl-6-(2-furyl)-9-(4-methoxyphenylmethyl)-9H-purine.

Anal: Found: C, 65.38; H, 4.66; N, 15.86. C₁₉H₁₆N₄O₃ requires C, 65.51; H, 4.63; N, 16.08.

6-(2-Furyl)-9-(4-methoxyphenylmethyl)-9H-purine-2-carbonitrile.

Anal: Found: C, 64.62; H, 4.15; N, 20.76. C₁₈H₁₃N₅O₂ requires C, 65.25; H, 3.95; N, 21.14.

2-Trimethylsilylethynyl-6-(2-furyl)-9-(4-methoxyphenylmethyl)-9H-purine.

Anal: Found: C, 65.66; H, 5.53; N, 13.88. C₂₂H₂₂N₄O₂Si requires C, 65.65; H, 5.51; N, 13.92.

2-Ethynyl-6-(2-furyl)-9-(4-methoxyphenylmethyl)-9H-purine.

Anal: Found: C, 69.26; H, 4.37; N, 16.71. C₁₉H₁₄N₄O₂ requires C, 69.08; H, 4.27; N, 16.96%.

6-(2-Furyl)-9-(4-methoxyphenylmethyl)-2-(1-propynyl)-9H-purine.

Anal: Found: C, 67.99; H, 4.71; N, 15.75. C₂₀H₁₆N₄O₂ requires C, 69.76; H, 4.68; N, 16.27%.

6-(2-Furyl)-9-(4-methoxyphenylmethyl)-2-phenyl-9H-purine.

Anal: Found: C, 72.27; H, 4.82; N, 14.73. C₂₃H₁₈N₄O₂ requires C, 72.24; H, 4.74; N, 14.65%.

2,6-(Di-2-furyl)-9-(4-methoxyphenylmethyl)-9H-purine.

Anal: Found: C, 67.89; H, 4.50; N, 14.89. C₂₁H₁₆N₄O₃ requires C, 67.73; H, 4.33; N, 15.05.

6-Chloro-9-(4-methoxyphenylmethyl)-8-methyl-9H-purine.

Anal: Found: 58.36; H, 4.44. C₁₄H₁₃ClN₄O requires C, 58.82; H, 4.54.

6,8-Dichloro-9-(4-methoxyphenylmethyl)-9H-purine.

Found: 50.24; H, 3.11; N, 17.83. C₁₃H₁₀Cl₂N₄O requires C, 50.50; H, 3.26; N, 18.12.

8-Bromo-6-Chloro-9-(4-methoxyphenylmethyl)-9H-purine.

Anal: Found: 43.88; H, 2.83; N, 15.89. C₁₃H₁₀BrClN₄O requires C, 44.16; H, 2.82; N, 15.85.

8-Bromo-6-(2-furyl)-9-(4-methoxyphenylmethyl)-9H-purine

Anal: Found: 53.25; H, 3.47; N, 14.04. C₁₇H₁₃BrN₄O₂ requires C, 53.01; H, 3.40; N, 14.54.

8-Chloro-6-(2-furyl)-9-(4-methoxyphenylmethyl)-9H-purine.

Anal: Found: 59.54; H, 3.84; N, 16.57. C₁₇H₁₃ClN₄O₂ requires C, 59.92; H, 3.84; N, 16.44.

6,8-Di(2-furyl)-9-(4-methoxyphenylmethyl)-9H-purine.

Anal: Found: 67.92; H, 4.44; N, 15.30. C₂₁H₁₆N₄O₃ requires C, 67.73; H, 4.33; N, 15.05.

8-Chloro-6-(5-chloro-2-furyl)-9-(4-methoxyphenylmethyl)-9H-purine.

Anal: Found: 54.87; H, 3.45; N, 15.18. C₁₇H₁₂Cl₂N₄O₂ requires C, 54.43; H, 3.22; N, 14.93.

6-(2-Furyl)-8-methoxy-9-(4-methoxyphenylmethyl)-9H-purine.

Anal: Found: 64.28; H, 4.69; N, 16.38. C₁₈H₁₆N₄O₃ requires C, 64.28; H, 4.80; N, 16.66.

6-(2-Furyl)-8-hydroxy-9-(4-methoxyphenylmethyl)-9H-purine.

Anal: Found: 63.28; H, 4.52; N, 17.15. C₁₇H₁₄N₄O₃ requires C, 63.35; H, 4.38; N, 17.38.

6-(2-Furyl)-8-methyl-9-(4-methoxyphenylmethyl)-9H-purine.

Found: 67.30; H, 5.02; N, 17.26. C₁₈H₁₆N₄O₂ requires C, 67.75; H, 5.22; N, 17.49.

### EXAMPLE 117

### Activity against Mycobacterium tuberculosis

The primary screening against Mycobacterium tuberculosis H₃₇Rv (ATCC 27294) was conducted at 6.25 μg/mL in BACTEC 12B medium using the Microplate Alamar Blue Assay (MABA). Compounds exhibiting fluorescence were tested in the BACTEC 460-radiometric system (see (a) Collins et al. Antimicrob. Agents Chemother, 1997, 41: 1004-1009 (1997); (b) http;//www.taacf.org/; (c) Orme et al. Antimicrob. Agents Chemother 45: 1943-1946 (2001)). Compounds demonstrating at least 90% inhibition in the primary screen were retested at lower concentrations against M. tuberculosis H₃₇Rv to determine the actual minimum inhibitory concentration (MIC) in the MABA.

The results are set out in Table 1 below.

TABLE 1

| Example No. | % inhibition at 6.25 μg/mL | MIC (μg/mL) |
|---|---|---|
| 27 | 94 | 6.25 |
| 28 | 95 | 6.25 |
| 29 | 91 | 6.25 |
| 30 | 52 | — |
| 31 | 95 | 3.13 |
| 32 | 98 | 12.5 |
| 33 | 97 | 1.56 |
| 34 | 98 | 6.25 |
| 35 | 99 | 0.78 |
| 36 | 8 | — |
| 37 | 88 | — |
| 38 | 89 | — |
| 39 | 97 | 3.13 |
| 40 | 99 | 1.56 |
| 41 | 79 | — |
| 42 | 98 | 6.25 |
| 43 | 91 | 12.5 |
| 44 | 0 | — |
| 45 | 23 | — |
| 46 | 98 | 1.56 |
| 47 | 98 | 6.25 |
| 48 | 99 | 3.13 |
| 49 | 96 | 3.13 |
| 50 | 98 | 0.78 |
| 51 | 98 | 0.39 |
| 52 | 99 | 3.13 |
| 53 | 99 | 0.78 |
| 54 | 98 | 1.56 |
| 55 | 98 | 0.78 |
| 57 | 97 | 0.39 |
| 61 | 60 | — |
| 65 | 5 | — |
| 68 | 98 | 0.78 |
| 69 | 60 | — |
| 70 | 0 | — |
| 72 | 56 | — |
| 74 | 97 | 0.78 |
| 76 | 99 | 0.20 |
| 77 | 99 | 1.56 |
| 78 | 60 | — |
| 79 | 97 | >6.25 |
| 80 | 62 | — |
| 81 | 96 | 6.25 |
| 82 | 99 | 3.13 |
| 83 | 91 | >6.25 |
| 84 | 99 | 6.25 |
| 85 | 37 | — |
| 86 | 97 | 6.25 |
| 87 | 69 | — |
| 89 | 97 | 6.25 |
| 94 | 82 | — |
| 98 | 93 | 6.25 |
| 99 | 90 | >6.25 |
| 101 | 40 | — |
| 102 | 72 | — |
| 103 | 0 | — |
| 104 | 95 | 3.13 |
| 105 | 64 | — |
| 107 | 86 | — |
| 109 | 94 | 0.39 |

### EXAMPLE 118

### Determination of 90% and 99% Effective Concentration in M. tuberculosis Infected Macrophages

Compounds were tested for the ability to kill M. tuberculosis Erdman (ATCC 35801) in monolayers of mouse bone marrow macrophages (see http://www.taacf.org/ and Skinner et al. Antimicrob. Agents Chemother 38; 2557-2563 (1994)) at 4-fold concentrations equivalent to 0.25, 1, 4 and 16 times the MIC. The results are presented in Table 2.

TABLE 2

| Example No. | EC⁺₉₉ (μg/mL) | EC₉₉ (μg/mL) | EC₉₀/MIC* |
|---|---|---|---|
| 33 | >12.5 | >12.5 | >8.01 |
| 51 | 0.02 | 0.67 | 0.05 |

*MIC data from Table 1
⁺ [symbol] is the concentration causing a n % reduction in residual mycobacterial growth after 7 days compared with untreated controls

1. An antimycobacterial 6-aryl-9-(m- or p-substituted-benzyl) purine, 8-aza-purine or 7-deaza-purine compound or a physiologically tolerable salt thereof.

2. A compound as claimed in claim 1 being a purine or 7-deaza-purine or a salt thereof.

3. A compound as claimed in claim 1 having a 4-methoxy-benzyl group at the 9-position.

4. A compound as claimed in claim 1 having a 2-furyl group at the 6-position.

5. A compound as claimed in claim 1 having a substituent at the 2-position containing 1 to 4 non-hydrogen atoms.

6. A pharmaceutical composition comprising an antimycobacterial 6-aryl-9-(m- or p-substituted-benzyl)-purine, 8-aza-purine or 7-deaza-purine or a physiologically tolerable salt thereof together with a physiologically tolerable carrier or excipient.

7. The use of an antimycobacterial 6-aryl-9-(m- or p-substituted-benzyl)-purine, 8-aza-purine or 7-deaza-purine or a physiologically tolerable salt thereof for the manufacture of a medicament for use in combatting mycobacterial infection, in particular tuberculosis.

8. A method of treatment of a human or non-human animal (e.g. a mammal) subject to combat mycobacterial infection, said method comprising administering to said subject an effective amount of an antimycobacterial 6-aryl-9-(m- or p-substituted-benzyl)-purine, 8-aza-purine or 7-deaza-purine or a physiologically tolerable salt thereof.

9. A method as claimed in claim 8 wherein said infection is tuberculosis.

10. A compound as claimed in claim 2 having a 4-methoxy-benzyl group at the 9-position.

11. A compound as claimed in claim 2 having a 2-furyl group at the 6-position.

12. A compound as claimed in claim 3 having a 2-furyl group at the 6-position.

13. A compound as claimed in claim 2 having a substituent at the 2-position containing 1 to 4 non-hydrogen atoms.

14. A compound as claimed in claim 3 having a substituent at the 2-position containing 1 to 4 non-hydrogen atoms.

15. A compound as claimed in claim 4 having a substituent at the 2-position containing 1 to 4 non-hydrogen atoms.
